(19) United States
(12) Patent Application Publication
(10) Pub. No.: US 2018/0037601 A1
(43) Pub. Date: Feb. 8, 2018

(54) Nucleoside-Based Anti-Bacterial and Anti-Protozoan Drugs

(71) Applicant: UTI Limited Partnership, Calgary (CA)

(72) Inventors: Thomas G. BACK, Calgary (CA); Floyd F. SNYDER, Calgary (CA)

(21) Appl. No.: 15/789,587

(22) Filed: Oct. 20, 2017

Related U.S. Application Data
(63) Continuation-in-part of application No. PCT/CA2016/000118, filed on Apr. 22, 2016.
(60) Provisional application No. 62/151,151, filed on Apr. 22, 2015.

(51) Int. Cl. C07H 19/16; C07H 19/14

(52) U.S. Cl. CPC C07H 19/16; C07H 19/14

(57) ABSTRACT

The present invention is directed to purine nucleoside analogs of the general formula I or salts and pharmaceutical compositions comprising such compounds and salts, which are useful as anti-protozoan agents. The invention is also directed to methods for treating a protozoan infection in a mammal and use of the compounds for inhibiting the growth of protozoa.

### CROSS-REFERENCE TO RELATED APPLICATIONS

This application is a continuation-in-part of International Patent Application PCT/CA2016/000118, filed Apr. 22, 2016, which claims the benefit of U.S. provisional application 62/151,151, filed Apr. 22, 2015. Each of these applications is incorporated by reference herein in its entirety.

### FIELD OF THE INVENTION

This invention relates to purine nucleoside analogs useful in the treatment of bacterial and protozoan infections. More particularly, the present invention relates to novel adenosine and guanosine analogs, the use of these compounds as pharmaceuticals, and pharmaceutical compositions containing the compounds.

### BACKGROUND OF INVENTION

Infectious diseases remain a serious global health problem. There has, for example, been a resurgence of tuberculosis, and the emergence of antibiotic-resistant strains of several key pathogens. Of particular concern is the increase in nosocomial infections. There is a clear need in the art for new anti-bacterial and anti-protozoan agents.

The protozoa are a diverse family of parasites that cause a considerable acute and chronic health burden. For example, the World Health Organization reported that Plasmodium falciparum, the causative agent of human malaria resulted in upwards of 600,000 deaths in 2012 (World Health Organization).¹ Trypanosoma brucei, the causal agent of African sleeping sickness, is transmitted by the tse-tse fly with cattle and wild game acting as reservoirs of human infective trypanosomes. The incidence of this disease is estimated at 500,000 cases per year and if left untreated is fatal.⁴ Trypanosoma cruzi, the causal agent of Chagas' disease, is endemic in certain areas of Central and South America. Though the pathology typically appears decades after initial infection and may result in sudden cardiac death, there are estimates of as many as 16 million persons infected with this parasite in these regions.⁵ Leishmania donovani is responsible for visceral and cutaneous leishmaniasis and is transmitted by sandflies. An estimated 12 million people are presently infected, with estimates of up to 50,000 deaths per year.⁶

Although for most of these protozoan parasitic diseases, there are safe and efficacious drugs available, resistant strains have emerged. For example, there is a history of P. falciparum progressively acquiring drug resistance. Resistance to chloroquine was widespread by the 1970's, requiring the modified drugs sulfadoxine-pyrimethamine and mefloquine.² The current drug of choice, artemisinin, is also being challenged with growing evidence for geographically widespread resistance.³

There has also been a significant increase in microbial antibiotic resistance. For example, methicillin-resistant Staphylococcus aureus (MRSA), with reduced susceptibility to vancomycin, the drug of choice for the treatment of MRSA, have been reported. Vancomycin-resistant Enterococcus faecalis (VRE) is also of concern. Of particular concern is the increase in hospital acquired (nosocomial) infections, for example, Pseudomonas aeruginosa, MRSA, and VRE account for 34% of all nosocomial infections. Another significant concern is drug-resistant Streptococcus pneumoniae (DRSP).

The combined impact of these disorders, coupled with progressive developing resistance, adverse side effects and even teratogenicity with some of the present generation of drugs, reflect the need for further drug development.

U.S. Pat. No. 7,084,127 reports C2,5′-disubstituted and N⁶′, C2,5′-trisubstituted adenosine derivatives of the general formula:

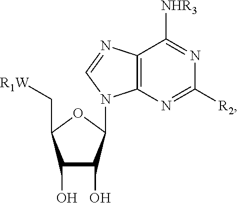

where variables are as defined therein, and their uses as adenosine receptor ligands. The compounds are disclosed as useful for treatment of certain diseases and disorders affected by adenosine receptor agonists, such as for antipsychotic drugs, and cardio- and cerebroprotective agents.

U.S. Published Patent Application No. 2008/0070860 reports purine nucleoside analogs of the general formula:

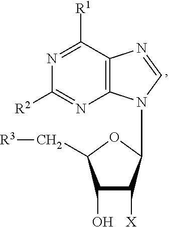

where variables are as defined therein, and their uses as anti-bacterial and anti-protozoan agents. This reference is incorporated by reference herein in its entirety for descriptions of compounds that are excluded from compound claims herein and for descriptions of synthetic and other methods that can be employed in preparation of compounds herein and in implementation of the methods herein.

### SUMMARY OF THE INVENTION

The present invention provides compounds of the general formula I:

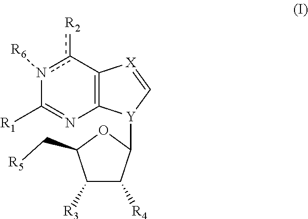

and salts thereof

wherein:

dashed lines represent potential bonds dependent upon R₂;

X and Y are independently selected from N, or CH;

R₁ is selected from the group consisting of a halogen, an amino group (—NH₂), an alkyl amino group (—N(R_{A})₂), an alkoxy group, a nitrosamino group (—N(R_{N})—NO), an imino group (—C(R_{C})═NR_{I}), an azide group, a cyano group, and a hydrazino group (—NH—N(R_{H})₂);

R₂ is selected from the group consisting of a halogen, oxo (═O), a sulfhydryl (—SH), an amino group (—NH₂), an alkyl amino group (—N(R_{A})₂), an alkoxy group, a thioalkyl group, a nitrosamino group (—N(R_{N})—NO), an imino group (—C(R₁₀)═NR_{I}), an azide group, a cyano group, a hydrazino group (—N(R₁₀)—N(R_{H})₂), a hydroxyamino group (—N(R_{HA})OH), a ureido group (—N(R₁₀)—CO—N(R_{U})₂), an amido group (—N(R₁₀)—CO—R_{AD}), alkyl sulfinyl (—SO—R_{S}),

a 1-(1H)pyrrolyl group:

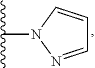 [SMILES: *n1cccn1]

a 1-(1H)-pyrazolyl group:

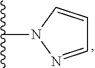 [SMILES: *n1cccn1]

and

a 1-(1H)-imidazolyl group;

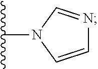 [SMILES: *n1ccnc1]

each R₃ and R₄, independently, is a hydroxyl, or an acyl group (—COR_{AC});

R₅ is selected from the group consisting of hydrogen, hydroxyl, an alkyl, an alkoxy, an azido group, a sulfoxide group (—SO—R_{SO}), a sulfonyl group (—SO₂—R_{SO}), an amino group (—NH₂), an alkyl amino group (—N(R_{A})₂, a thioalkyl group (—SR_{T}), an amido group (—N(R₁₀)—CO—R_{AD}),

an N-phthalimido group:

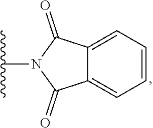 [SMILES: *N1C(=O)c2ccccc2C1=O]

and

a morpholino group:

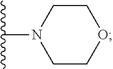 [SMILES: *N1CCOCC1]

and

R₆ is present when R₂ is oxo and is selected from the group consisting of hydrogen or an alkyl group having 1-3 carbon atoms;

where

each R_{A}, R_{S} or R_{SO} is independently selected from an alkyl group having 1-6 carbon atoms, a cycloalkyl group having 3-6 carbon atoms, or an alkyl group having 1-3 carbon atoms, or more specifically a methyl, ethyl, propyl or a cyclopropyl group; and

each R₁₀, R_{C}, R_{H}, R_{I}, R_{HA}, R_{AD}, R_{AC}, R_{N}, R_{U}, and R_{T} is independently selected from hydrogen, an alkyl group having 1-3 carbon atoms, a cycloalkyl group having 3-6 carbon atoms or an alkyl having 1-3 carbon atoms, or more specifically a methyl, ethyl, propyl or a cyclopropyl group.

In a specific embodiment, the invention provides the compounds of formula 1 with the exception that X and Y are not both N.

In a specific embodiment, the invention provides the compounds of formula 1 with the exception that X and Y are not both CH.

In a specific embodiment, the invention provides the compounds of formula 1 with the exception that R₅ is not OH.

More specific embodiments of the invention include those having the formulas IA or IB:

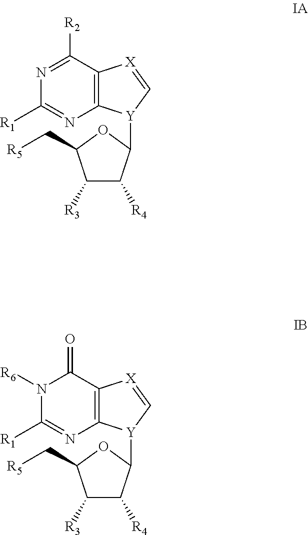

and salts thereof,

wherein Y and X are independently CH or N and the other variables are as defined for formula I.

In formula IA, R₂ is other than oxo. In specific embodiments of formula IA or IB, Y is N and R₆ is hydrogen. In other embodiments of formulas IA and IB, X is N and R₆ is hydrogen. In specific embodiment of formula IA, when both X and Y are N, R₁ is other than —NH₂, R₂ is other than hydrogen, or R₃, R₄ or R₅ is other than —OH. In specific embodiments of formula IB, when both X and Y are N, R₁ is other than —NH₂, R₆ is other than hydrogen, or R₃, R₄ or R₅ is other than —OH. In specific embodiments of formulas IB and IA, when both X and Y are N, R₁ is a halogen, an alkoxy group, a nitrosamino group (—N(R_{N})—NO), an imino group (—C(R_{C})═NR_{I}), an azide group, a cyano group, or a hydrazino group (—NH—N(R_{H})₂.

In other specific embodiments, the present invention provides compounds of formulas IIA, IIB, IIC or IID

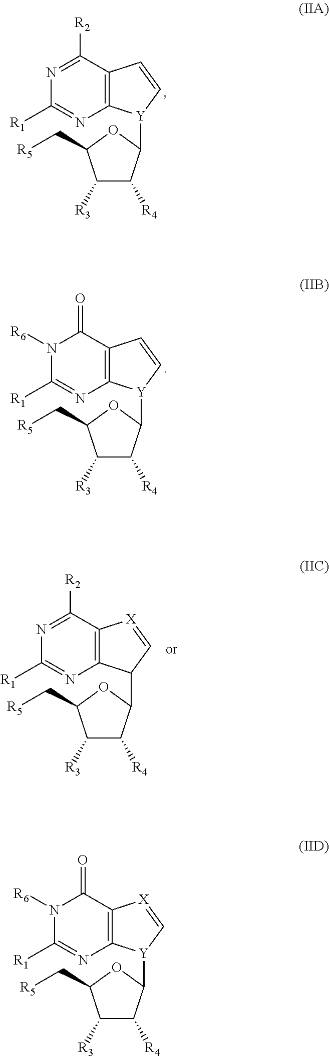

or salts thereof,

where Y and X are independently N or CH and other variables are as in formula I. In formulas IIA and IIC, R₂ is other than oxo. In specific embodiments of formulas IIB and IIID, Y is N or X is N and R₆ is hydrogen. In specific embodiments of formulas IIA-IID, Y is N. In specific embodiments of formulas IIA-IID, Y is CH. In specific embodiments of formulas IIB and IIID, R₆ is hydrogen. In specific embodiment of formulas IIB and IIID, R₁ is other than —NH₂, or R₃, R₄ or R₅ is other than —OH. In specific embodiments of formulas IIA and IICB, R₁ is other than —NH₂, or at least one of R₂, R₃, R₄ or R₅ is other than —OH. In specific embodiments of formulas IIA-IID, R₁ is a halogen, an alkoxy group, a nitrosamino group (—N(R_{N})—NO), an imino group (—C(R_{C})═NR_{I}), an azide group, a cyano group, or a hydrazino group (—NH—N(R_{H})₂. In specific embodiments of formulas IIA or IIC, R₂ is halogen.

In other specific embodiments, the present invention provides compounds of formulas IIIA, IIIB, and IIIC:

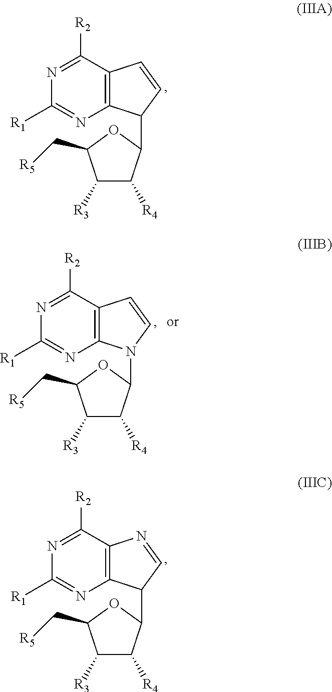

or salts thereof, where variables are as defined for formula I, except that R₂ is not oxo. In specific embodiments of formulas IIIA-IIIC R₁ is other than —NH₂, R₂ is other than hydrogen or or at least one of R₃, R₄ or R₅ is other than —OH. In specific embodiments of formulas IIIA-IIIC, R₁ is a halogen, an alkoxy group, a nitrosamino group (—N(R_{N})—NO), an imino group (—C(R_{C})═NR_{I}), an azide group, a cyano group, or a hydrazino group (—NH—N(R_{H})₂. In specific embodiments of formulas IIIA-IIIC, R₂ is halogen. In specific embodiments of formulas IIIA-IIIC, R₁ is an amino group and R₂ is other than hydrogen. In specific embodiments of formulas IIIA-IIIC, R₂ is hydrogen and R₁ is a halogen, an alkoxy group, a nitrosamino group (—N(R_{N})—NO), an imino group (—C(R_{C})═NR_{I}), an azide group, a cyano group, or a hydrazino group (—NH—N(R_{H})₂. In specific embodiments of formulas IIIA-IIIC, R₂ is halogen and R₁ is a halogen, an alkoxy group, a nitrosamino group (—N(R_{N})—NO), an imino group (—C(R_{C})═NR_{I}), an azide group, a cyano group, or a hydrazino group (—NH—N(R_{H})₂.

In other specific embodiments, the present invention provides compounds of formulas IIIE, IIIF, or IIIG:

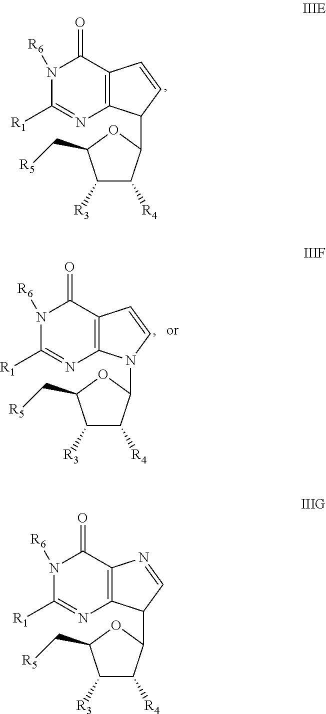

and salts thereof, where variables are as defined above for formula I. In specific embodiments of formulas IIIE-IIIG, R₆ is hydrogen. In specific embodiments of formulas IIIE-IIIG, R₆ is other than hydrogen. In specific embodiments, R₁ is —NH₂, and R₆ is other than hydrogen, or at least one of R₃, R₄ or R₅ is other than —OH. In specific embodiments of formulas IIIE-IIIG, R₁ is a halogen, an alkoxy group, a nitrosamino group (—N(R_{N})—NO), an imino group (—C(R_{C})═NR_{I}), an azide group, a cyano group, or a hydrazino group (—NH—N(R_{H})₂.

In other specific embodiments, the present invention provides compounds of formulas IVA, or IVB:

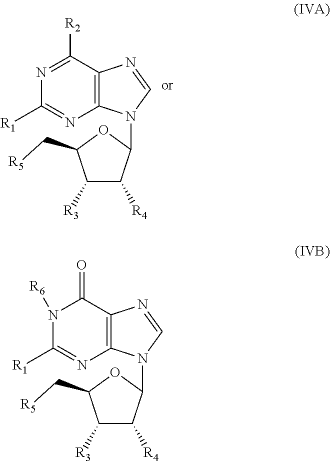

or salts thereof where variables are as defined for formula I, except that R₂ is not oxo in formula IVA. In specific embodiments of formulas IVA or IVB, R1 is other than —NH₂, or at least one of R₃, R₄ or R₅ is other than OH. In specific embodiments of formula IVB, R₆ is other than hydrogen. In specific embodiments of formula IVB, R₆ is hydrogen and at least one of R₃, R₄ or R₅ is other than OH or R₁ is other than —NH₂. In specific embodiments of formulas IVA or IVB, R₁ is a halogen, an alkoxy group, a nitrosamino group (—N(R_{N})—NO), an imino group (—C(R_{C})═NR_{I}), an azide group, a cyano group, or a hydrazino group (—NH—N(R_{H})₂).

In other specific embodiments, the present invention provides compounds of formulas VA, VB or VC:

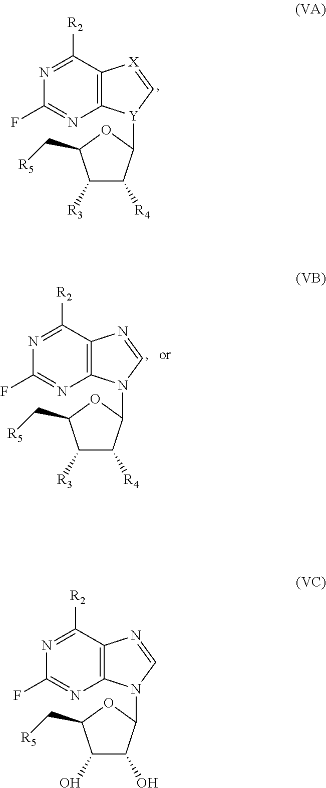

or a salt thereof;

wherein variables are as defined for formula I. In specific embodiments of formula VA, X is N and Y is CH. In specific embodiments of formula VA, X is CH and Y is N. In specific embodiments of formula VA, both of X and Y are CH.

In further embodiments of formulas VA-VC, R₂ is selected from an alkyl amino group (—N(R_{A})₂), an azide group, a hydrazino group (—N(R₁₀)—N(R_{H})₂), a hydroxyamino group (—N(R_{HA})OH), a ureido group (—N(R₁₀)—CO—N(R_{U})₂), an amido group (—N(R₁₀)—CO—R_{AD}), an alkyl sulfinyl (—SO—R_{S}) group, a 1-(1H)pyrrolyl group, a 1-(1H)-pyrazolyl group, and a 1-(1H)-imidazolyl group.

In further specific embodiments of formulas VA-VC, R₅ is hydrogen, thioalkyl, or more specifically thiomethyl.

In other specific embodiments, the present invention provides compounds of formulas VIA, VIB or VIC:

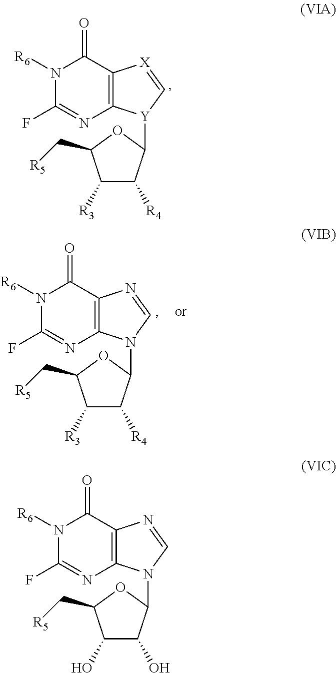

or salts thereof, wherein variables are as defined for formula I. In specific embodiments of formula VIA, X is N and Y is CH. In specific embodiments of formula VIA, X is CH and Y is N. In specific embodiments of formula VIA, both of X and Y are CH.

In further embodiments of formulas VIA-VIC, R₆ is hydrogen. In further embodiments of formulas VIA-VIC, R₆ is other than a hydrogen. In further specific embodiments of formulas VIA-VIC, R₅ is hydrogen, thioalkyl, or more specifically thiomethyl.

In other specific embodiments, the present invention provides compounds of formulas VIIA, VIIB, VIIC, VIID, VIIE, or VIIF:

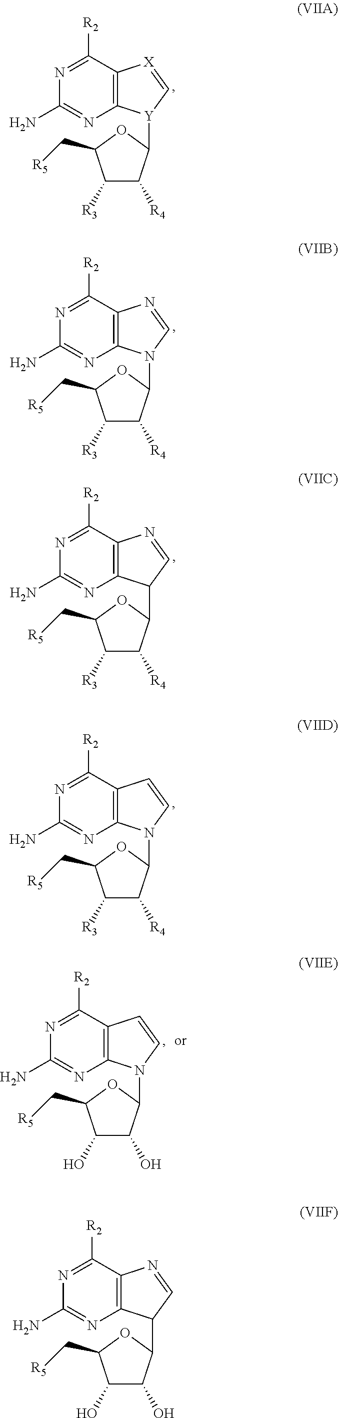

or a salt thereof;

wherein variables are as defined for formula I. In specific embodiments of formula VIIA, X and Y are both CH. In specific embodiments of formulas VIIA-VIIF, R₂ is other than hydrogen.

In further embodiments of formulas VIIA-VIIF, R₂ is selected from an alkyl amino group (—N(R_{A})₂), an azide group, a hydrazino group (—N(R₁₀)—N(R_{H})₂), a hydroxyamino group (—N(R_{HA})OH), a ureido group (—N(R₁₀)—CO—N(R_{U})₂), an amido group (—N(R₁₀)—CO—R_{AD}), an alkyl sulfinyl (—SO—R_{S}) group, a 1-(1H)pyrrolyl group, a 1-(1H)-pyrazolyl group, and a 1-(1H)-imidazolyl group.

In further specific embodiments of formulas VA-VC, R₅ is hydrogen, thioalkyl, or more specifically thiomethyl.

In specific embodiments of formula I, R₂ is a 1-(1H)pyrrolyl group, a 1-(1H)-pyrazolyl group, and a 1-(1H)-imidazolyl group. In specific embodiments of formulas IA, IIA, IIC, IIIA-IIIC, IVA, VA-VA, or VIIA-VIIF, R₂ is a 1-(1H)pyrrolyl group, a 1-(1H)-pyrazolyl group, or a 1-(1H)-imidazolyl group. In specific embodiments of formula I, R₂ is selected from an azide group, or a hydrazino group (—N(R₁₀)—N(R_{H})₂). In specific embodiments of formula I, R₂ is selected from a hydroxyamino group (—N(R_{HA})OH), or a ureido group (—N(R₁₀)—CO—N(R_{U})₂). In specific embodiments of formula I, R₂ is an amido group (—N(R₁₀)—CO—R_{AD}). In specific embodiments of formula I, R₂ is an alkyl sulfinyl (—SO—R_{S}) group. In specific embodiments of formulas IA, IIA, IIC, IIIA-IIIC, IVA, VA-VA, or VIIA-VIIF, R₂ is selected from an azide group, or a hydrazino group (—N(R₁₀)—N(R_{H})₂). In specific embodiments of formulas IA, IIA, IIC, IIIA-IIIC, IVA, VA-VA, or VIIA-VIIF, R₂ is selected from a hydroxyamino group (—N(R_{HA})OH), or a ureido group (—N(R₁₀)—CO—N(R_{U})₂). In specific embodiments of formulas IA, IIA, IIC, IIIA-IIIC, IVA, VA-VA, or VIIA-VIIF, R₂ is an amido group (—N(R₁₀)—CO—R_{AD}). In specific embodiments of formulas IA, IIA, IIC, IIIA-IIIC, IVA, VA-VA, or VIIA-VIIF, R₂ is an alkyl sulfinyl (—SO—R_{S}) group.

In other specific embodiments, the present invention provides compounds of formulas VIIIA-VIIID:

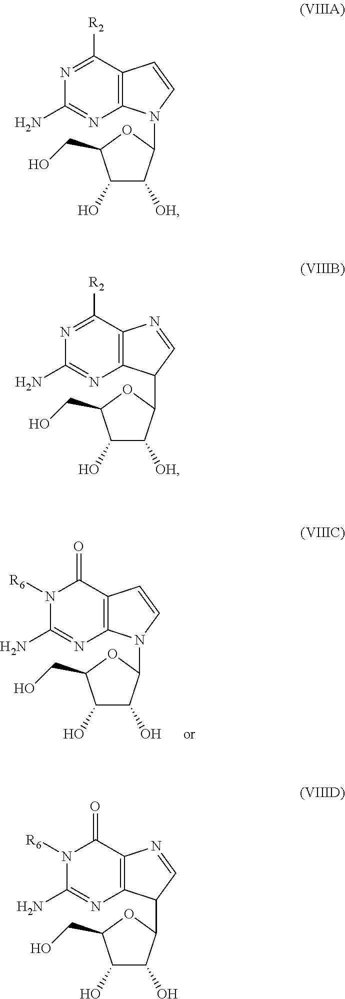

or a salt thereof;

wherein the variables are defined as in formula I. except that for formulas VIIIA and VIIIB, R₂ is not oxo. In specific embodiments of formulas VIIIA and VIIIB, R₂ is a halogen or a sulfhydryl group. In specific embodiments of formulas VIIIC and VIIID, R₆ is other than a hydrogen.

In other specific embodiments, the present invention provides compounds of formulas IXA and IXB:

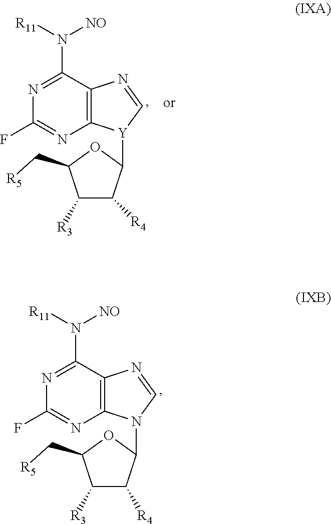

or a salt thereof;

wherein R₁₁ is an alkyl group having 1-6 carbon atoms or an alkyl group having 1-3 carbon atoms, or a methyl group, and other variables are as defined for formula I.

In specific embodiments of formula IXA, Y is CH and X is N.

In specific embodiments of formula IXA, Y is N and X is CH.

In specific embodiments of formulas IXA and IXB, both of R₃ and R₄ are hydroxyl.

In specific embodiments of formulas IXA and IXB, R₁₁ is a methyl group.

In specific embodiments of formulas IXA and IXB, R₅ is a thioalkyl group.

In specific embodiments of formulas IXA and IXB, both of R₃ and R₄ are hydroxyl and R₅ is a thioalkyl group.

In specific embodiments of formulas IXA and IXB, both of R₃ and R₄ are hydroxyl and R₅ is a thiomethyl group.

In specific embodiments of formulas IXA and IXB, both of R₃ and R₄ are hydroxyl, R₅ is a thiomethyl group and R₁₁ is an alkyl group.

In specific embodiments of formulas IXA and IXB, both of R₃ and R₄ are hydroxyl, R₅ is a thiomethyl group and R₁₁ is a methyl group.

In other specific embodiments, the present invention provides compounds of formulas XA or XB:

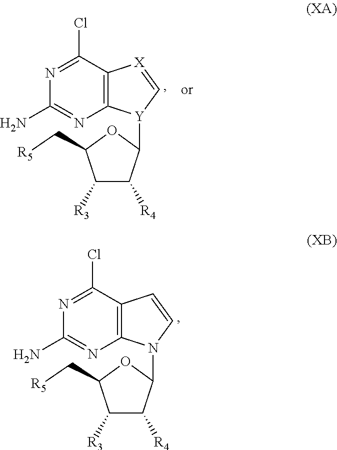

or a salt thereof;

wherein the variables are as defined for formula I.

In specific embodiments of formula XA, Y is CH and X is N.

In specific embodiments of formula XA, Y is N and X is CH.

In specific embodiments of formula XA, Y is N and X is N.

In specific embodiments of formulas XA and XB, both of R₃ and R₄ are hydroxyl.

In specific embodiments of formulas XA and XB, R₅ is a hydroxyl group.

In specific embodiments of formulas XA and XB, both of R₃ and R₄ are hydroxyl and R₅ is a hydroxyl.

The invention also provides a method of treating a protozoan infection in a mammal comprising administering a therapeutically effective amount of any one or more of the compounds of formulas I, or compounds of any other of the above formulas or compounds of each specific embodiment defined above; or a tautomer thereof; or a physiologically acceptable salt or solvate thereof; or a prodrug thereof.

The invention also provides a method of treating a bacterial infection in a mammal comprising administering a therapeutically effective amount of any one or more of the compounds of formulas I, or compounds of any other of the above formulas or compounds of each specific embodiment defined above; or a tautomer thereof; or a physiologically acceptable salt or solvate thereof; or a prodrug thereof.

In another aspect of the invention, there is provided a method of inhibiting the growth of a protozoa comprising contacting the protozoa with a growth-inhibiting effective amount of any one or more of the compounds of formulas I or compounds of any other of the above formulas or compounds of each specific embodiment defined above; or a tautomer thereof; or a physiologically acceptable salt or solvate thereof; or a prodrug thereof.

In another aspect of the invention, there is provided a method of inhibiting the growth of bacteria comprising administering a growth-inhibiting effective amount of any one or more of the compounds of formulas I, or compounds of any other of the above formulas or compounds of each specific embodiment defined above; or a tautomer thereof; or a physiologically acceptable salt or solvate thereof; or a prodrug thereof.

The invention further provides a pharmaceutical composition for the treatment of protozoan infection comprising as active ingredient(s) any one or more compounds of any of the formulas I, or compounds of any other of the above formulas or compounds of each specific embodiment defined above; or a tautomer thereof; or a physiologically acceptable salt or solvate thereof; or a prodrug thereof.

The invention further provides a pharmaceutical composition for the treatment of a bacterial infection comprising as active ingredient(s) any one or more compounds of any of the formulas I, or compounds of any other of the above formulas or compounds of each specific embodiment defined above; or a tautomer thereof; or a physiologically acceptable salt or solvate thereof; or a prodrug thereof.

Additional embodiments of the invention will be apparent to one of ordinary skill in the art in view of the drawings, detailed description and examples herein.

### BRIEF DESCRIPTION OF THE DRAWINGS

FIGS. 1A and 1B. Pathways of nucleoside reutilization exploited in the present invention. Cleavage of 6-aminopurine ribonucleosides is shown in (FIG. 1A). Conversion of inosine or guanosine to the corresponding nucleotide is shown in (FIG. 1B).

FIG. 2. Summary table of in vitro antiprotozoan activity and cytotoxicity of nucleoside analogs.

### DETAILED DESCRIPTION OF THE INVENTION

The present invention is based on the development of purine nucleoside analogs useful for treatment of bacterial and/or protozoan infections. Zheng et al. (2016) (reference 73) and Tran et al. (2017) (reference 74) are incorporated by reference herein in their entirety at least for any additional details with respect to the synthesis of compounds of the invention, methods of assessing antimicrobial activity of compounds of the invention and activities of compounds of the invention.

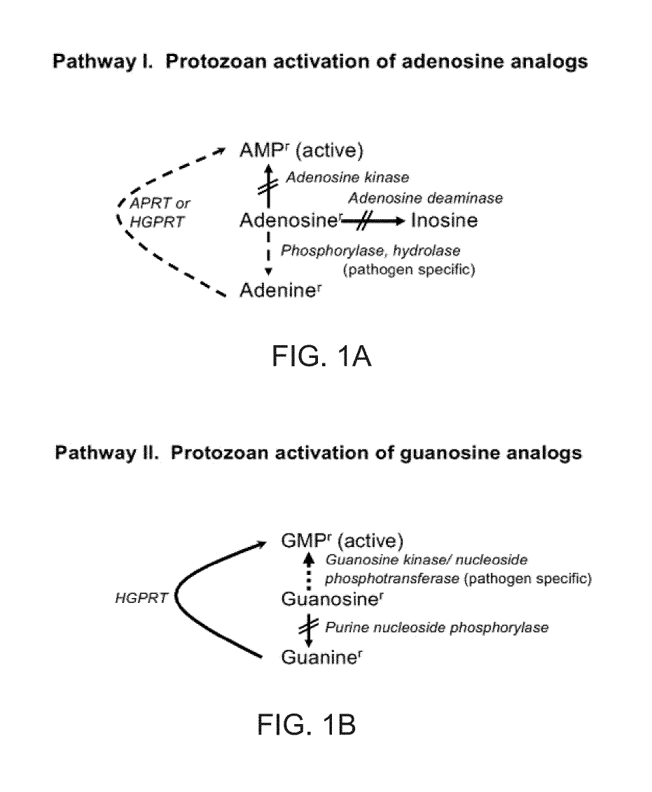

Protozoa are universally deficient in their capability to synthesize purines via a de novo pathway,⁷⁻⁹ resulting in their dependence upon the reutilization of preformed purines for development and proliferation. Protozoa have several unique enzymes not present in mammals, which are exploitable targets for drug design. The present description provides designs of purine ribonucleoside prodrug analogues which are activated in the parasite, but which remain comparatively inert in the mammalian host. As these activation pathways are essential for the proliferation of the protozoa, the exploitable targets may be more refractory to mutagenesis than conventional targets. Nucleoside analogue strategies herein have focused at least on two processes of nucleoside reutilization present in the parasite but not in mammals (FIGS. 1A and 1B).

### Pathway I.

Certain adenine ribonucleoside analogues were designed to exploit the unique ability of protozoan parasites to cleave 6-aminopurine ribonucleosides (FIG. 1A), a reaction which is not carried out in mammals. This enzymatic transformation is predominantly effected by the protozoan inosine-adenosine-guanosine nucleoside hydrolase, IAG-NH.¹⁰⁻¹¹ The sequence identity of the IAG-NH proteins for T. vivax, T.b. brucei, T. congolense and L. major is greater than 50%.¹² An adenine nucleoside phosphorylase has been partially purified from Schistosoma mansoni which catalyzes the phosphorolysis of adenosine and 5′-methylthioadenosine, but not inosine and guanosine,¹³ with evidence for broader distribution.¹⁴ For Pathway I, following nucleoside cleavage to the corresponding purine base, the analogue is converted to the active nucleotide through the action of the purine phosphoribosyltransferases APRT or HGXPRT.

Adenosine is not a substrate for P. falciparum PNP^{15, 16} or the mammalian PNP,^{17,18} and the IAG-NH is absent in P. falciparum.¹⁴ Although adenosine does not directly undergo phosphorolysis in P. falciparum, this parasite has a unique adenosine deaminase¹⁹ which converts both adenosine and methylthioadenosine, the product of polyamine catabolism, to inosine and methylthioinosine. The Pf PNP accepts both nucleosides and converts them to hypoxanthine,^{16, 19} which can be reutilized for nucleotide synthesis via the reaction catalyzed via HGXPRT. Since 2-chloro and 2-fluoropurine ribonucleosides are toxic to bacteria,^{20, 21} the halogenated adenosine analogues are expected to generate toxic metabolites when cleaved by enzymes that are present in bacteria and protozoa, but absent in mammals.

To further enhance the specificity of certain adenosine analogues, they must also be protected from direct activation to a toxic form by the host adenosine kinase through modification of the 5′-hydroxyl group (FIG. 1A). A further route of metabolism and potential activation in the host would be through deamination to the inosine analogue, which could then be acted upon by mammalian purine nucleoside phosphorylase and the resultant purine base could be converted to an active nucleotide via hypoxanthine phosphoribosyltransferase activity. Substitution of the 2-position of the purine ring with the halogens fluorine or chlorine is also known to block deamination via mammalian adenosine deaminase.^{22,23} Thus, certain of the present nucleoside-based antiprotozoan drugs are designed to be toxic to the pathogen through their conversion to 2-chloro or 2-fluoropurines, while remaining refractory to deamination and phosphorylation in host cells and thus preventing the formation of toxic metabolites.

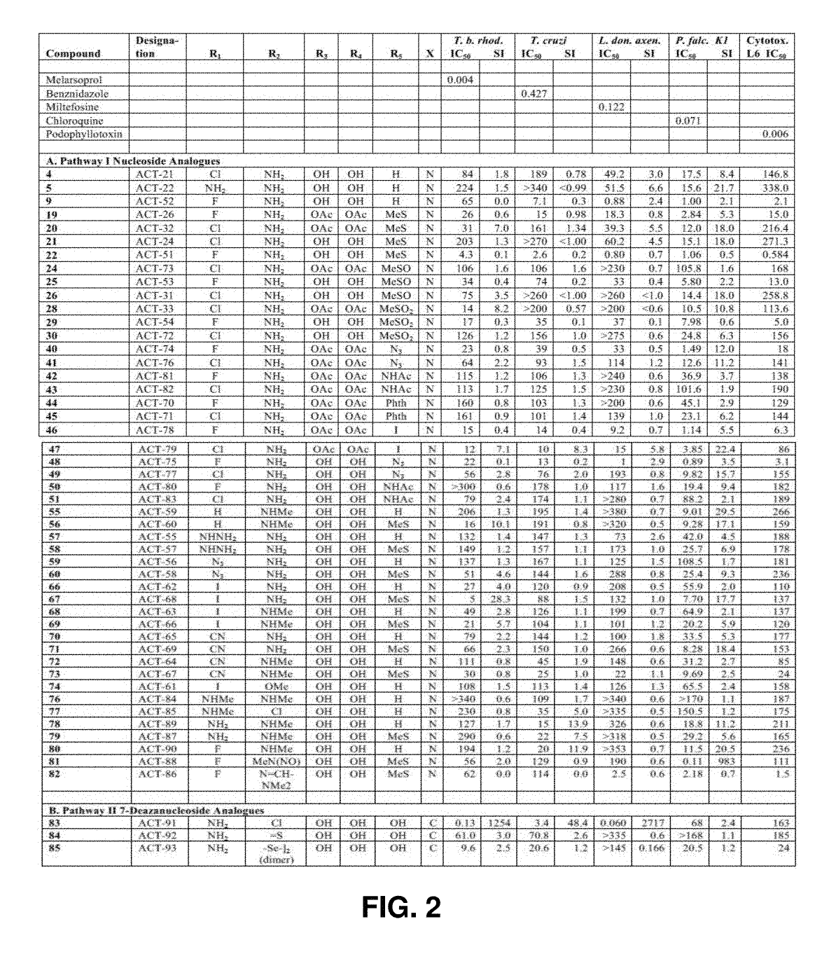

Of the 47 adenosine analogues tested in this category, 15 had a selectivity index of greater than 10 toward P. falciparum, relative to their cytotoxicity to a mammalian cell line (L6 rat myoblast cells). One compound had a selectivity index of 983 and IC₅₀ in the nanomolar range (FIG. 2).

### Pathway II.

Protozoa have the unique capacity to directly convert inosine or guanosine to the corresponding nucleotide, whereas mammals lack this activity (FIG. 1B). The nucleoside may be converted in a single step to an active nucleotide via a guanosine kinase as described for Trichomonas vaginalis,²⁴ or more broadly via a nucleoside phosphotransferase.²⁵ Phosphorolysis to the corresponding base via the host mammalian purine nucleoside phosphorylase may be blocked by modifications at the purine 7 and 9 positions. The 9-deaza C—C glycoside analogs are refractory to cleavage to the free purine base analogue and ribose. This is important to prevent base analogues from being activated via HGPRT in the host. Similarly, as protonation of the N-7 position is involved in the transition state facilitating phosphorolysis or hydrolysis, the C-7 analogs are also not cleaved.^{16, 26, 27} Inosine-guanosine nucleosides with these modifications are inert in the mammalian host and activated by phosphorylation in the parasite.

Previous studies have shown the 7-deaza- and 9-deaza-inosine analogues have demonstrated activity against L. Donovani,^{28,29} Trypanosoma brucei rhodesiense,³⁰ T.b. gambiense,³⁰ L. Mexicana²⁹ and Giardia³¹. There is also direct evidence for their conversion to the corresponding nucleoside triphosphates^{28,29} and the formycin B (9-deaza-8-aza-inosine) monophosphate inhibited the activity of adenylosuccinate synthetase,²⁹ thereby preventing conversion of IMP to adenine nucleotides.

Three compounds were tested in this category. One exhibited IC₅₀'s in the nanomolar range against T.b. rhodesiense and L. donovani with selectivity indices of 1250 and 2720, respectively. One of these compounds was also active against T. cruzi in the micromolar range with a selectivity index of 48 (FIG. 2). In specific embodiments, compounds of the present invention are useful for treatment against infections by protozoa including, but not limited to those of the genera Plasmodium, Cryptosporidium, Acanthanmoeba Trypanosoma, Leishmania, Schistosoma, Trichomomas, Entamoeba, or Giardia

In specific embodiments, compounds of the present invention are useful for treatment against infections by protozoa including, but not limited to Plasmodium falciparum, P. berghei, P. malariae, P. vivax, P. ovale, Crytosporidium sp., Cryptosporidium parvum, C. hominis, Acanthanmoeba spp. A. culbertsoni, A. polyphaga, A. castellanii, A. astronyxis, A. hatchetti, A. rhysodes, A. divionensis, A. lugdunensis, A. lenticulata, Trypanosoma brucei brucei, T.b. rhodesiense, T.b. gambiense, T. cruzi, T. vivax, T. congolense, Leishmania donovani, L. major, L. mexicana, L. tropica, L. braziliensis, Schistosoma mansoni, Trichomomas vaginalis, Entamoeba invadens or Giardia lamblia.

The invention provides methods of treatment of infections by administration of a therapeutically effective amount of one or more compounds of formula I. Administration can be orally, by injection, rectally, intravaginally, intranasally or by local application. Methods include treatment of mammals and more specifically treatment of humans. Depending on the specific condition or disease state to be treated, subjects may be administered compounds of the present invention at any suitable therapeutically effective and safe dosage, as may be readily determined within the skill of the art. These compounds are, most desirably, administered in dosages ranging from about 1 to about 1000 mg per day, in a single dose or divided doses, although variations will necessarily occur depending upon the weight and condition of the subject being treated and the particular route of administration chosen. However, a dosage level that is in the range of about 1 to about 250 mg/kg, preferably between about 5 and 100 mg/kg, is most desirable. Variations may nevertheless occur depending upon the weight and conditions of the persons being treated and their individual responses to said medicament, as well as on the type of pharmaceutical formulation chosen and the time period and interval during which such administration is carried out. In some instances, dosage levels below the lower limit of the aforesaid range may be more than adequate, while in other cases still larger doses may be employed without causing any harmful side effects, provided that such large doses are first divided into several small doses for administration throughout the day.

The invention specifically includes veterinary applications of the one or more compounds of the invention for the treatment of protozoan infections. One of ordinary skill in the art can determine appropriate veterinary formulations for treatment of various non-human mammals without resort to undue experimentation.

In an embodiment, compounds of the present invention can be administered in the form of any pharmaceutical formulation or dosage form which comprises a therapeutically effective amount of one or more compounds of the invention in combination with a pharmaceutically acceptable carrier, the nature of which will depend upon the route of administration. The dosage form can for example be a solid or liquid dosage form. The dosage form can be a solution or suspension comprising one or more active ingredients. The dosage form can be a pharmaceutically acceptable aqueous solution. These pharmaceutical compositions can be prepared by conventional methods, using compatible, pharmaceutically acceptable excipients or vehicles. Examples of such compositions include capsules, tablets, transdermal patches, lozenges, troches, sprays, syrups, powders, granulates, gels, elixirs, suppositories, injectable preparations, or preparations for rectal, nasal, ocular, vaginal, etc. administration, and the like.

A specific route of administration is oral administration. For oral administration, tablets containing various excipients such as microcrystalline cellulose, sodium citrate, calcium carbonate, dicalcium phosphate and glycine may be employed along with various disintegrants such as starch (preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc can be used for tableting purposes. Solid compositions of similar type may also be employed as fillers in gelatin capsules; preferred materials in this connection also include lactose or milk sugar, as well as high molecular weight polyethylene glycols. When aqueous suspensions and/or elixirs are desired for oral administration the active ingredient may be combined with sweetening or flavoring agents, coloring matter and, if so desired, emulsifying and/or suspending agents, together with such diluents as water, ethanol, propylene glycol, glycerine and various combinations thereof.

The dosage form can be designed for immediate release, controlled release, extended release, delayed release or targeted delayed release. The definitions of these terms are known to those skilled in the art. Furthermore, the dosage form release profile can be effected by a polymeric mixture composition, a coated matrix composition, a multiparticulate composition, a coated multiparticulate composition, an ion-exchange resin-based composition, an osmosis-based composition, or a biodegradable polymeric composition. Without wishing to be bound by theory, it is believed that the release may be effected through favorable diffusion, dissolution, erosion, ion-exchange, osmosis or combinations thereof.

For parenteral administration, for example, a solution of an active compound in either sesame or peanut oil or in aqueous propylene glycol can be employed. The aqueous solutions should be suitably buffered (preferably pH greater than 8), if necessary, and the liquid diluent first rendered isotonic. The aqueous solutions are suitable for intravenous injection purposes. The preparation of all these solutions under sterile conditions is readily accomplished by standard pharmaceutical techniques well known to those skilled in the art.

In specific embodiments, compounds of the present invention are useful for treatment against infections by bacteria including, but not limited to, Gram negative and Gram positive bacteria. In other embodiments, compounds of the present invention are useful for treatment against infections by the bacteria of the genus Escherichia, Shigella, Salmonella, Yersinia, Klebsiella, Pasteurella, Actinobacillus, Vibrio, Shewanella, Buchnera, Helicobacler, Bacillus, Listeri, Lactococcus, Clostridium, Enterococcus, Streptococcus, Streptococcus, Pseudomonas and Staphylococcus. In other embodiments, compounds of the present invention are useful for treatment against infections by the bacteria Escherichia coli, Shigella flexneri, Salmonella enterica serovar Typhi, Salmonella typhimurium, Yersinia pestis, Klebsiella sp., Pasteurella mullocida, Actinobacillus pleuropneumoniae, Vibrio cholera, Shewanella oneidensis, Buchnera sp., Helicobacler pylori, Bacillus subtilus, Listeria innocua, Listeria monocylogenes, Lactococcus lactis cremonis, Closlridium perfringens, Enterococcus faecium, Pseudomonas aeuriginosa, Pseudomonas cepia, and Streptococcus pneumonia and more specifically against Escherichia coli K-I2, Escherichia coli 01571H7.

### Definitions

In general, the terms and phrases used herein have their art-recognized meaning, which can be found by reference to standard texts, journal references and contexts known to those skilled in the art. The following definitions are provided to clarify their specific use in the context of the invention.

The term “alkyl” refers to a monovalent, saturated hydrocarbon group, either linear or branched. Preferred alkyl groups have from 1 to 6 carbon atoms. Specific preferred alkyl groups include: methyl, ethyl, n-propyl, iso-propyl, n-butyl, iso-butyl, sec-butyl, t-butyl, n-pentyl or isomers thereof, n-hexyl or isomers thereof. A more preferred alkyl group is a methyl group.

The term “cycloalkyl” refers to a monovalent, saturated cyclic hydrocarbon group having 3-6 ring carbon atoms. Non-limiting examples of cycloalkyl groups include cyclopropyl, cyclobutyl, cyclopentyl, or cyclohexyl and the like. A more preferred cycloalkyl group is cyclopropyl.

The term “treatment”, as used herein, includes administering a therapeutic amount of the compound or composition of the present invention which is effective to alleviate, ameliorate, or abate a disease or condition (e.g., an infection) or one or more symptoms thereof, to prevent additional symptoms, to inhibit a disease or condition, or to lessen the severity or cure a disease or condition.

The term “pharmaceutical composition” refers to the active ingredient (e.g. the compound of formula I, and physiologically acceptable salts thereof) together with one or more pharmaceutically acceptable carriers thereof and optionally other therapeutic ingredients.

The term “pharmaceutically acceptable,” as used herein, refers to a material that is safe and non-toxic for in vivo, preferably, human administration.

The term “carrier”, as used herein, refers to relatively non-toxic chemical compounds or agents that facilitate the incorporation of a compound into cells or tissues.

The term “therapeutically effective amount” refers to an amount that is effective for the treatment of a specific condition or disorder by administration of a compound, mixture of compounds or composition described herein.

In an embodiment, the compounds of formula I and salts and solvates thereof can be used in manufacture of a medicament for the treatment of infections caused by one or more of the protozoa noted herein. In an embodiment, the invention provides use of one or more of the compounds of formula I for the treatment of infection by caused by one or more of the protozoa noted herein.

In an embodiment, the compounds of any of formulas IA, IB, IIA-IID, IIIA-IIIG, IVA, IVB, VA-VC, VIA-VIC, VIIA-VIIF, VIIIA-VIIID, IXA, IXB, XA and XB and salts and solvates thereof can be used in manufacture of a medicament for the treatment of infections caused by one or more of the protozoa noted herein. In an embodiment, the invention provides use of one or more of the compounds of formula I for the treatment of infection by caused by one or more of the protozoa noted herein.

In embodiments herein, methods for inhibition of the growth of a protozoan are provided which employ a growth-inhibiting effective amount of one or more compounds of the invention. A growth-inhibiting effective amount is the amount or combined amount of one or more compounds of the invention which when added to an environment containing the protozoan induce a measurable decrease in growth of the organism in the environment compared to growth of the organism in the same environment without the presence of the one or more compounds. A growth-inhibiting effective amount includes without limitation an amount which effects killing of the organism in the environment.

In embodiments herein, methods for inhibition of the growth of a bacterium are provided which employ a growth-inhibiting effective amount of one or more compounds of the invention. A growth-inhibiting effective amount is the amount or combined amount of one or more compounds of the invention which when added to an environment containing the bacterium induce a measurable decrease in growth of the organism in the environment compared to growth of the organism in the same environment without the presence of the one or more compounds. A growth-inhibiting effective amount includes without limitation an amount which effects killing of the organism in the environment. Bacteria which can be inhibited include Gram positive bacteria. Bacteria which can be inhibited include Gram negative bacteria.

The term “salt”, as used herein, is any physiologically acceptable salt. A physiologically acceptable salt is any non-toxic alkali metal, alkaline earth metal, and ammonium salts commonly used in the pharmaceutical industry, including the sodium, potassium, lithium, calcium, magnesium, barium ammonium and protamine zinc salts, which are prepared by methods known in the art. The term also includes non-toxic acid addition salts, which are generally prepared by reacting the compounds of this invention with a suitable organic or inorganic acid. The acid addition salts are those which retain the biological effectiveness and properties of the free bases and which are not biologically or otherwise undesirable. Examples include acids derived from mineral acids, and include, inter alia, hydrochloric, hydrobromic, sulfuric, nitric, phosphoric, metaphosphoric and the like. Organic acids include, inter alia, tartaric, acetic, propionic, citric, malic, malonic, lactic, fumaric, benzoic, cinnaminc, mandelic, glycolic, gluconic, pyruvic, succinic, salicylic and arylsulphonic, e.g. p-toluenesulphonic, acids. Salts of the invention may be in the form of hydrates or solvates, where for example the molar ratio of water or solvent to salt can range from 0.5 to 10, or 0.5 to 6. The term “prodrug” refers to a compound that may be converted under physiological conditions or by solvolysis to a biologically active compound of the present application. It is a metabolic precursor of a compound of the present application that is pharmaceutically acceptable. A prodrug may be inactive when administered to a subject in need thereof, but is converted in vivo to an active compound of the present application. Prodrugs are typically rapidly transformed in vivo to yield the parent compound of the present application, for example, by hydrolysis in blood. Salts can be in the form of a hydrate.

The term “solvate” as used herein is a combination, or physical association of a compound with a solvent molecule. A specific solvate is a hydrate. Solvates can include those where the molar ratio of solvent to compound ranges, for example from/2 to 10 or more typically ½ to 4 and can include a disolvate, monosolvate or hemisolvate, among others. This physical association can involve varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. In a specific embodiment, solvates are isolatable with one or more molecules of solvent incorporated into the crystal lattice of a crystalline solid. Compounds of the invention may be present as solvated forms with a pharmaceutically acceptable solvent, such as water, methanol, ethanol, and the like, and it is intended that the invention includes both solvated and unsolvated forms of compounds of the invention. Solvates typically can function as pharmacological equivalents. Solvates typically do not significantly alter the physiological activity or toxicity of the compounds. Preparation of solvates is known in the art. See, for example, M. Caira et al., J. Pharmaceut. Sci., 93(3):601-611 (2004), E. C. van Tonder et al., AAPS Pharm. Sci. Tech., 5(1): Article 12 (2004), and A. L. Bingham et al., Chem. Commun.: 603-604 (2001). A typical, non-limiting, process of preparing a solvate would involve dissolving a compound of the invention in a desired solvent, which may be water, an organic solvent or a mixture thereof at temperatures above about 20° C., e.g. at room temperature or heating to a temperature above room temperature if appropriate to dissolve the solid, followed by cooling the solution at a rate sufficient to form crystals, and isolating crystals by known methods. Well-known analytical methods, such as infrared spectroscopy, can be used to confirm the presence of the solvent in a crystal of the solvate. More generally, a compound of the invention can be recrystallized from an appropriate solvent (e.g., water) to obtain a solvate (e.g., a hydrate). The term “tautomer” refers to a proton shift from one atom of a molecule to another atom of the same molecule. The present application includes tautomers of any said compound.

The term “selectivity index,” as used herein, refers to the relative cytotoxicity of a compound in a protozoan or bacterium versus the cytotoxicity of that compound in a mammal, expressed as the ratio [IC₅₀ (mammal)]/IC₅₀ (protozoan). or [IC₅₀ (mammal)]/IC₅₀ (bacterium)].

In certain embodiments of the invention, a compound of formula I

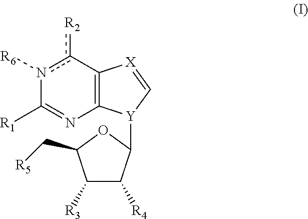

or salts and or solvates thereof are provided

wherein:

X is N and Y is N; or X is CH and Y is N; or X is N and Y is CH; or X is CH and Y is CH; and

R₁ is a halogen, an amino group (—NH₂), an alkyl amino group (—N(R_{A})₂), an alkoxy group, a nitrosamino group (—N(R_{N})—NO), an imino group (—C(R_{C})═NR_{I}), an azide group, a cyano group, or a hydrazino group (—NH—N(R_{H})₂); and

R₂ is a halogen, an oxo (═O), a sulfhydryl (—SH), an amino group (—NH₂), an alkyl amino group (—N(R_{A})₂), an alkoxy group, a thioalkyl group, a nitrosamino group (—N(R_{N})—NO), an imino group (—C(R₁₀)═NR_{I}), an azide group, a cyano group, a hydrazino group (—N(R₁₀)—N(R_{H})₂), a hydroxyamino group (—N(R_{HA})OH), a ureido group (—N(R₁₀)—CO—N(R_{U})₂), an amido group (—N(R₁₀)—CO—R_{AD}), alkyl sulfinyl (—SO—R_{S}), a 1-(1H)pyrrolyl group, a 1-(1H)-pyrazolyl group, or a 1-(1H)-imidazolyl group; when R₂ is oxo, R₆ is present;

each R₃ and R₄, independently, is a hydroxyl, or an acyl group (—COR_{AC}); and

R₅ is a hydrogen, a hydroxyl, an alkyl, an alkoxy, an azido group, a sulfoxide group (—SO—R_{SO}), a sulfonyl group (—SO₂—R_{SO}), an amino group (—NH₂), an alkyl amino group (—N(R_{A})₂, a thioalkyl group (—SR_{T}), an amido group (—N(R₁₀)—CO—R_{AD}), an N-phthalimido group, or a morpholino group; and wherein

each R_{A}, R_{S} or R_{SO} is independently selected from an alkyl group having 1-6 carbon atoms or cycloalkyl group having 3-6 carbon atoms, or an alkyl group having 1-3 carbon atoms, or more specifically a methyl, ethyl, propyl or a cyclopropyl group; and

each R₁₀, R_{C}, R_{H}, R_{I}, R_{HA}, R_{AD} or R_{AC} is independently selected from hydrogen, an alkyl group having 1-6 carbon atoms or cycloalkyl group having 3-6 carbon atoms, or an alkyl group having 1-3 carbon atoms, or more specifically a methyl, ethyl, propyl or a cyclopropyl group.

In a specific embodiment, the invention is directed to pharmaceutically acceptable salts of the compounds of any one of formulas I, IA, IB, IIA-IID, IIIA-IIIG, IVA, IVB, VA-VC, VIA-VIC, VIIA-VIIF, VIIIA-VIIID, IXA, IXB, XA and XB or a compound of each of specific embodiments defined herein.

In a specific embodiment, the invention is directed to pharmaceutically acceptable solvates of the compounds of any one of formulas I, IA, IB, IIA-IID, IIIA-IIIG, IVA, IVB, VA-VC, VIA-VIC, VIIA-VIIF, VIIIA-VIIID, IXA, IXB, XA and XB or a compound of each of specific embodiments defined herein.

In a specific embodiment, the invention is directed to pharmaceutically acceptable hydrates of the compounds of any one of formulas I, IA, IB, IIA-IID, IIIA-IIIG, IVA, IVB, VA-VC, VIA-VIC, VIIA-VIIF, VIIIA-VIIID, IXA, IXB, XA and XB, or a compound of each of specific embodiments defined herein.

In a specific embodiment, the invention is directed to the compounds of any one of formulas I, IA, IB, IIA-IID, IIIA-IIIG, IVA, IVB, VA-VC, VIA-VIC, VIIA-VIIF, VIIA-VIIID, IXA, IXB, XA and XB, or a compound of each of specific embodiments defined herein.

In an embodiment, compounds of the invention include pharmaceutically acceptable salts or solvates thereof of any formula herein which retain the physiologic activity of the corresponding free base or acid. The salts and free base or acid forms of the compounds of the invention may be different in some physical properties, such as, solubility. See: for example, S. M. Berge, et al., “Pharmaceutical Salts,” J. Pharm. Sci., 66: 1-19 (1977), which is incorporated by reference herein for teachings with respect to salts and solvates thereof. The compounds of the present invention or pharmaceutically acceptable salts thereof can form solvates, such as hydrates, or alcoholates.

In an embodiment, compounds, salts and solvates of the invention are provided which exhibit a selectivity index of greater than 5. In another embodiment, compounds, salts and solvates of the invention are provided which exhibit a selectivity index of greater than 20. In another embodiment, compounds, salts and solvates of the invention are provided which exhibit a selectivity index of greater than 100. In another embodiment, compounds, salts and solvates of the invention are provided which exhibit a selectivity index of greater than greater than 500.

In an embodiment, compounds, salts, and solvates are provided which exhibit an IC₅₀ for inhibition of a protozoa of 100 nM or less. In an embodiment, compounds, salts, and solvates are provided which exhibit an IC₅₀ for inhibition of a protozoa of 500 nM or less. In an embodiment, compounds, salts, and solvates are provided which exhibit an IC₅₀ for inhibition of a protozoa of 1 mM or less.

In an embodiment, compounds, salts, and solvates are provided which exhibit an IC₅₀ of 100 nm or less for inhibition of P. falciparum.

In an embodiment, compounds, salts, and solvates are provided which exhibit an IC₅₀ of 100 nm or less for inhibition of T. brucei.

In an embodiment, the invention provides methods for treating protozoan infections and the symptoms associated therewith.

In an embodiment, the invention provides methods for treating bacterial infections and the symptoms associated therewith.

The invention provides pharmaceutical compositions comprising a pharmaceutically effective amount of one or more compounds and/or salts of formula I or any other formula herein and a pharmaceutically acceptable carrier or excipient. The compounds and salts thereof of the invention can be used to prepare medicaments for the treatment of infectious diseases and disorders and the symptoms associated therewith.

The term “pharmaceutically effective amount” refers to an amount effective for treatment of a infection in an individual (human or other mammal) in need of such treatment either by administration of a single compound or salt of formula I or in combination with other agents. The pharmaceutically effective amount of a given compound when administered as the only active ingredient may differ from its pharmaceutically effective amount when administered with other active ingredients. It will be appreciated that the pharmaceutically effective amount of a compound may differ from that of a salt of the same compound. Treating includes the alleviation of symptoms of a particular disorder in a patient or a measurable improvement of a parameter associated with a particular disorder.

As used herein, the term “individual” includes reference to a mammal, including a human.

Compounds of the invention of formula I can be administered in the form of pharmaceutically acceptable salts which include the following non-limiting examples: alkali metal salts, such as those of lithium, potassium and sodium; alkali earth metal salts, such as those of barium, calcium and magnesium; transition metal salts, such as those of zinc; and other metal salts, such as those of aluminum, sodium hydrogen phosphate and disodium phosphate; salts of nitrates, borates, methanesulfonates, benzene sulfonates, toluenesulfonates, salts of mineral acids, such as those of hydrochlorides, hydrobromides, hydroiodides and sulfates; salts of organic acids, such as those of acetates, trifuoroacetates, maleates, oxalates, lactates, malates, tartrates, citrates, benzoates, salicylates, ascorbates, succinates, butyrates, valerates and fumarates. amine salts, such as those of N,N′-dibenzylethylenediamine, chloroprocaine, choline, ammonia, diethanolamine and other hydroxyalkylamines, ethylenediamine, N-methylglucamine, procaine, N-benzylphenethylamine, I-para-chlorobenzyl-2-pyrrolidin-1′-ylmethyl-benzimidazole, diethylamine and other alkylamines, piperazine and tris(hydroxymethyl)-aminomethane.

Pharmaceutically acceptable salts can be derived from inorganic or organic acids or can be derived from inorganic or organic bases as is known in the art. Basic amino acids useful for salt formation include arginine, lysine and ornithine. Acidic amino acids useful for salt formation include aspartic acid and glutamic acid. Compound of the invention can be administered in the form of pharmaceutically acceptable esters which include, among others, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids and boronic acids.

Compounds and salts of the invention in the form of pharmaceutical compositions or dosage forms the invention can be administered by any known route that is appropriate for the individual being treated and for the treatment or prophylaxis that is desired. Specifically administration can be orally or non-orally in the form of, for example, granules, powders, tablets, capsules, syrup, suppositories, injections, emulsions, elixir, suspensions or solutions, by mixing these effective components, individually or simultaneously, with pharmaceutically acceptable carriers, excipients, binders, diluents or the like.

A solid formulation for oral administration can comprise one or more of the compounds or salts of the invention alone or in appropriate combination with other active ingredients. Solid formulations can be in the form of powders, granules, tablets, pills and capsules. In these cases, the instant compounds can be mixed with at least one additive, for example, sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. These formulations can contain, as in conventional cases, further additives, for example, an inactive diluent, a lubricant such as magnesium stearate, a preservative such as paraben or sorbic acid, an anti-oxidant such as ascorbic acid, tocopherol or cysteine, a disintegrator, a binder, a thickening agent, a buffer, a sweetener, flavoring agent and/or a perfuming agent. Tablets and pills can also be prepared with enteric coating. Standard methods of formulation can be applied to preparation of formulations of the compounds and salts of the invention.

Non-oral administration includes subcutaneous injection, intravenous injection, intramuscular injections, intraperitoneal injection or instillation. Injectable preparations, for example, sterile injectable aqueous suspensions or oil suspensions can be prepared by known methods.

The instant pharmaceutical compositions may be formulated as known in the art for nasal aerosol or inhalation and may be prepared as solutions in saline, and benzyl alcohol or other suitable preservatives, absorption promoters, fluorocarbons, or solubilizing or dispersing agents.

Rectal suppositories can be prepared by mixing the drug with a suitable vehicle, for example, cocoa butter and polyethylene glycol, which is in the solid state at ordinary temperatures, in the liquid state at temperatures in intestinal tubes and melts to release the drug. Examples of liquid preparations for oral administration include pharmaceutically acceptable emulsions, syrups, elixirs, suspensions and solutions, which may contain an inactive diluent, for example, pharmaceutically acceptable water.

The pharmaceutical composition can be formulated for topical administration, for example, with a suitable ointment containing one or more of the compounds or salts of the invention suspended or dissolved in a carrier, which include mineral oil, liquid petroleum, white petroleum, propylene glycol, polyoxyethylene polyoxypropylene compound, emulsifying wax and pharmaceutically acceptable water. In addition, topical formulations can be formulated with a lotion or cream containing the active compound suspended or dissolved in a carrier. Suitable carriers include mineral oil, sorbitan monostearate, polysorbate 60, cetyl esters wax, cetearyl alcohol, 2-octyldodecanol, benzyl alcohol and pharmaceutically acceptable water.

As is understood in the art, dosages of the instant compounds are dependent on age, body weight, general health conditions, sex, diet, dose interval, administration routes, excretion rate, combinations of drugs and conditions of the diseases treated. While taking these and other necessary factors into consideration, generally, dosage levels of between about 10 pg per day to about 5000 mg per day, preferably between about 100 mg per day to about 1000 mg per day of the compound are useful in the prevention and treatment of fibrotic diseases or disorders. Typically, the pharmaceutical compositions of this invention will be administered from about 1 to about 5 times per day or alternatively, as a continuous infusion. Such administration can be used as a chronic or acute therapy.

The amount of active ingredient that may be combined with the carrier or excipient materials to produce a single dosage form will vary depending upon the patient/individual treated and the particular mode of administration. A typical preparation will contain from about 5% to about 95% active compound (W/W). Preferably, such preparations contain from about 20% to about 80% active compound. While these dosage ranges can be adjusted by a necessary unit base for dividing a daily dose, as described above, such doses are decided depending on the diseases to be treated, conditions of such diseases, the age, body weight, general health conditions, sex, diet of the patient then treated, dose intervals, administration routes, excretion rate, and combinations of drugs. While taking these and other necessary factors into consideration., for example, a typical preparation will contain from about 0.05% to about 95% active compound (W/W). Preferably, such preparations contain from about 10% to about 80% active compound. The desired unit dose of the composition of this invention is administered once or multiple times daily.

In an embodiment, compounds of the invention include pharmaceutically acceptable salts or solvates thereof of formula I or any other formula herein, which retain the physiologic activity of the corresponding free base or acid. The salts and free base or acid forms of the compounds of the invention may be different in some physical properties, such as, solubility. See: for example, S. M. Berge, et al., “Pharmaceutical Salts,” J. Pharm. Sci., 66: 1-19 (1977), which is incorporated by reference herein for teachings with respect to salts and solvates thereof. The compounds of the present invention or pharmaceutically acceptable salts thereof can form solvates, such as hydrates, or alcoholates. Methods are known in the art for making solvates and particularly hydrates of compounds and salts. Salts of the invention can be in the form of solvates and particularly in the form of hydrates.

In specific embodiments of any one of the formulas herein,

R₂ is oxo and R₆ is present;
R₂ is other than oxo and R₆ is not present;

### X is N and Y is N;

### X is CH and Y is N;

### X is N and Y is CH; or

### X is CH and Y is CH.

In specific embodiments of any formulas herein and in further embodiments of any one of the previously recited embodiments of X and Y:

R₁ is a halogen,
R₁ is an amino group (—NH₂),
R₁ is an alkyl amino group (—N(R_{A})₂),
R₁ is an alkoxy group,
R₁ is a nitrosamino group (—N(R_{N})—NO),
R₁ is an imino group (—C(R_{C})=NR_{I}),
R₁ is an azide group,
R₁ is a cyano group, or
R₁ is a hydrazino group (—NH—N(RH)₂).

In specific embodiments of any one of the formulas herein and in further embodiments of any one of the previously recited embodiments of X, Y and R₁:

R₂ is a halogen,
R₂ is a sulfhydryl (—SH),
R₂ is an amino group (—NH₂)
R₂ is an alkyl amino group (—N(R_{A})₂),
R₂ is an alkoxy group,
R₂ is a thioalkyl group,
R₂ is a nitrosamino group (—N(R_{N})—NO),
R₂ is an imino group (—C(R₁₀)=NR_{I}),
R₂ is an azide group,
R₂ is a cyano group,
R₂ is a hydrazino group (—N(R₁₀)—N(RH)₂),
R₂ is a hydroxyamino group (—N(R_{HA})OH),
R₂ is a ureido group (—N(R₁₀)—CO—N(R_{U})₂),
R₂ is an amido group (—N(R₁₀)—CO—R_{AD}),
R₂ is an alkyl sulfinyl (—SO—R_{S}),
R₂ is a 1-(1H)pyrrolyl group,
R₂ is a 1-(1H)-pyrazolyl group, or
R₂ is a 1-(1H)-imidazolyl group.

In specific embodiments of any one of the formulas herein and in further embodiments of any one of the previously recited embodiments of X, Y, R₁: or R₂:

each R₃ and R₄, independently, is a hydroxyl, or an acyl group,
each R₃ and R₄ is hydroxyl;
each R₃ and R4 is an acyl group, or
one of R₃ or R₄ is hydroxyl and the other of R₃ or R₄ is an acyl group.

In specific embodiments of any one of the formulas herein and in further embodiments of any one of the previously recited embodiments of X, Y, R₁, R₂, R₃: or R₄:

R₅ is a hydrogen,
R₅ is a hydroxyl,
R₅ is an alkyl,
R₅ is an alkoxy,
R₅ is an azido group,
R₅ is a sulfoxide group (—SO—R_{SO}),
R₅ is a sulfonyl group (—SO₂—R_{SO}),
R₅ is an amino group (—NH₂),
R₅ is an alkyl amino group (—N(R_{A})₂,
R₅ is a thioalkyl group (—SR_{T}),
R₅ is an amido group (—N(R₁₀)—CO—R_{AD}),
R₅ is an N-phthalimido group, or
R₅ is a morpholino group.

In specific embodiments of any one of the formulas herein and in further embodiments of any one of the previously recited embodiments of X, Y, R₁, R₂, R₃, R₄ or R₅:

each R_{A}, R_{S} or R_{SO} is independently selected from an alkyl group having 1-6 carbon atoms or a cycloalkyl group having 3-6 carbon atoms,
each R_{A}, R_{S} or R_{SO} is independently selected from an alkyl group having 1-3 carbon atoms,
each R_{A}, R_{S} or R_{SO} is independently selected from a methyl, ethyl, propyl or a cyclopropyl group; or
each R_{A}, R_{S} or R_{SO} is a methyl group.

In specific embodiments of any one of the formulas herein wherein R₂ is OXO and in further embodiments of any one of the previously recited embodiments of X, Y, R₁, R₃, R₄, R₅, R_{A}, R_{S} or R_{SO}:

R₆ is a hydrogen;
R₆ is other than a hydrogen;
R₆ is an alkyl group having 1-6 carbon atoms;
R₆ is an alkyl group having 1-3 carbon atoms; or
R₆ is a methyl group.

In specific embodiments of any one of the formulas herein and in further embodiments of any one of the previously recited embodiments of X, Y, R₁, R₂, R₃, R₄, R₅, R_{A}, R_{S}, R_{SO}, or R₆ (when R₂ is oxo):

each R₁₀, R_{C}, R_{H}, R_{I}, R_{HA}, R_{AD} or R_{AC} is independently selected from hydrogen, an alkyl group having 1-6 carbon atoms,
each R₁₀, R_{C}, R_{H}, R_{I}, R_{HA}, R_{AD} or R_{AC} is independently selected from hydrogen or an alkyl group having 1-3 carbon atoms or a cycloalkyl group having 3-6 carbon atoms,
each R₁₀, R_{C}, R_{H}, R_{I}, R_{HA}, R_{AD} or R_{AC} is independently selected from an alkyl group having 1-3 carbon atoms,
each R₁₀, R_{C}, R_{H}, R_{I}, R_{HA}, R_{AD} or R_{AC} is independently selected from a methyl, ethyl, propyl or a cyclopropyl group,
each R₁₀, R_{C}, R_{H}, R_{I}, R_{HA}, R_{AD} or R_{AC} is hydrogen or a methyl group, or
each R₁₀, R_{C}, R_{H}, R_{I}, R_{HA}, R_{AD} or R_{AC} is hydrogen.

In specific embodiments of the forgoing embodiments, a salt of the compound of any one of the formulas herein is provided.

In specific embodiments of the forgoing embodiments, a solvate of the compound of any one of the formulas herein is provided.

In specific embodiments of the forgoing embodiments, a hydrate of the compound of any one of the formulas herein is provided.

In specific embodiments of formula I, when R₂ is a monoalkyl amino group and R₁ is a halogen, then R₅ is a group other than alkoxy or thioalkyl. In other specific embodiments of formula I, when R₁ is a halogen and R₅ is an alkoxy or thioalkyl group, then R₂ is a group other than a monoalkyl amino group. In other specific embodiments of formula I, when R₂ is a monoalkyl amino group and R₅ is an alkoxy or thioalkyl group, then R₁ is a group other than a halogen.

In specific embodiments of formula I, when X=Y=N, R₂ is a monoalkyl amino group and R₁ is a halogen, then R₅ is a group other than alkoxy or thioalkyl. In other specific embodiments of formula I, when X=Y=N, R₁ is a halogen and R₅ is an alkoxy or thioalkyl group, then R₂ is a group other than a monoalkyl amino group. In other specific embodiments of formula I, when X=Y=N, R₂ is a monoalkyl amino group and R₅ is an alkoxy or thioalkyl group, then R₁ is a group other than a halogen.

In specific embodiments of formula I, when R₂ is an amino group and R₁ is a halogen, then R₅ is a group other than alkoxy or thioalkyl. In other specific embodiments of formula I, when R₁ is a halogen and R₅ is an alkoxy or thioalkyl group, then R₂ is a group other than an amino group. In other specific embodiments of formula I, when R₂ is an amino group and R₅ is an alkoxy or thioalkyl group, then R₁ is a group other than a halogen.

In specific embodiments of formula I, when X=Y=N, R₂ is an amino group and R₁ is a halogen, then R₅ is a group other than alkoxy or thioalkyl. In other specific embodiments of formula I, when X=Y=N, R₁ is a halogen and R₅ is an alkoxy or thioalkyl group, then R₂ is a group other than an amino group. In other specific embodiments of formula I, when X=Y=N, R₂ is a monoalkyl amino group and R₅ is an alkoxy or thioalkyl group, then R₁ is a group other than a halogen.

In specific embodiments of formula I, R₂ is a group other than an amino group. In specific embodiments of formula I, R₂ is a group other than a monoalkylamino group. In specific embodiments of formula I, R₅ is a group other than alkoxy or thioalkyl. In specific embodiments of formula I, R₁ is a group other than a halogen.

In specific embodiments of formula I, when X=Y=N, R₄ is hydroxyl, R₁ is halogen or amino, R₂ is amino, sulfhydryl, alkoxy or thioalkyl, then R₅ is other than a hydrogen, an amino group, an alkoxy group, or an alkyl group. In other specific embodiments of formula I, when X=Y=N, R₄ is hydroxyl, R₁ is halogen or amino, and R₅ is other than a hydrogen, an amino group, an alkoxy group, or an alkyl group, then R₂ is a group other than amino, sulfhydryl, alkoxy or thioalkyl, In other specific embodiments of formula I, when X=Y=N, R₄ is hydroxyl, R₅ is a hydrogen, an amino group, an alkoxy group, or an alkyl group, and R₂ is an amino, sulfhydryl, alkoxy or thioalkyl group, then R₁ is a group other than a halogen or amino group.

In specific embodiments of formula I, when R₄ is hydroxyl, R₁ is halogen or amino, R₂ is amino, sulfhydryl, alkoxy or thioalkyl, then R₅ is other than a hydrogen, an amino group, an alkoxy group, or an alkyl group. In other specific embodiments of formula I, when R₄ is hydroxyl, R₁ is halogen or amino, and R₅ is other than a hydrogen, an amino group, an alkoxy group, or an alkyl group, then R₂ is a group other than amino, sulfhydryl, alkoxy or thioalkyl, In other specific embodiments of formula I, when R₄ is hydroxyl, R₅ is a hydrogen, an amino group, an alkoxy group, or an alkyl group, and R₂ is an amino, sulfhydryl, alkoxy or thioalkyl group, then R₁ is a group other than a halogen or amino group.

In specific embodiments of formula I, when R₁ is halogen or amino, R₂ is amino, sulfhydryl, alkoxy or thioalkyl, then R₅ is other than a hydrogen, an amino group, an alkoxy group, or an alkyl group. In other specific embodiments of formula I, when R₄ is hydroxyl, R₁ is halogen or amino, and R₅ is other than a hydrogen, an amino group, an alkoxy group, or an alkyl group, then R₂ is a group other than amino, sulfhydryl, alkoxy or thioalkyl, In other specific embodiments of formula I, when R₄ is hydroxyl, R₅ is a hydrogen, an amino group, an alkoxy group, or an alkyl group, and R₂ is an amino, sulfhydryl, alkoxy or thioalkyl group, then R₁ is a group other than a halogen or amino group.

Compounds of the invention, salts thereof and solvates thereof can be synthesized in view of the methods provided herein and further in view of methods and techniques which are well-known to one of ordinary skill in the art. Methods herein can, for example, be readily adapted by choice of starting material, reagent and/or solvent for the synthesis of compounds of the invention. Methods for preparation of salts and solvates and particularly for hydrates are well-known in the art and can be readily applied to prepare salts and solvates of the compounds of any one of the formulas herein.

Compounds of the present invention, and salts thereof, may exist in their tautomeric form, in which hydrogen atoms are transposed to other parts of the molecules and the chemical bonds between the atoms of the molecules are consequently rearranged. It should be understood that all tautomeric forms, insofar as they may exist, are included within the invention. Additionally, inventive compounds may have trans and cis isomers and may contain one or more chiral centers, therefore exist in enantiomeric and diastereomeric forms. The invention includes all such isomers, as well as mixtures of cis and trans isomers, mixtures of diastereomers and racemic mixtures of enantiomers (optical isomers). When no specific mention is made of the configuration (cis, trans or R or S) of a compound (or of an asymmetric carbon), then any one of the isomers or a mixture of more than one isomer is intended. The processes for preparation can use racemates, enantiomers, or diastereomers as starting materials. When enantiomeric or diastereomeric products are prepared, they can be separated by conventional methods, for example, by chromatographic or fractional crystallization. The inventive compounds may be in the free or hydrate form.

With respect to the various compounds of the invention, the atoms therein may have various isotopic forms, e.g., isotopes of hydrogen include deuterium and tritium. All isotopic variants of compounds of the invention are included within the invention and particularly included at deuterium and ¹³C isotopic variants. It will be appreciated that such isotopic variants may be useful for carrying out various chemical and biological analyses, investigations of reaction mechanisms and the like. Methods for making isotopic variants are known in the art.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group are intended to be individually included in the disclosure. Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomer and enantiomer of the compound described individually or in any combination. One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the disclosure. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### Example 1: Synthesis Procedures and Characterization

The compounds of the present invention may be prepared by several synthesis procedures. For example, the synthesis route to obtain the 2-chloro- and 2-fluoradenosines modified at the 5′ position are depicted in scheme 1 below:

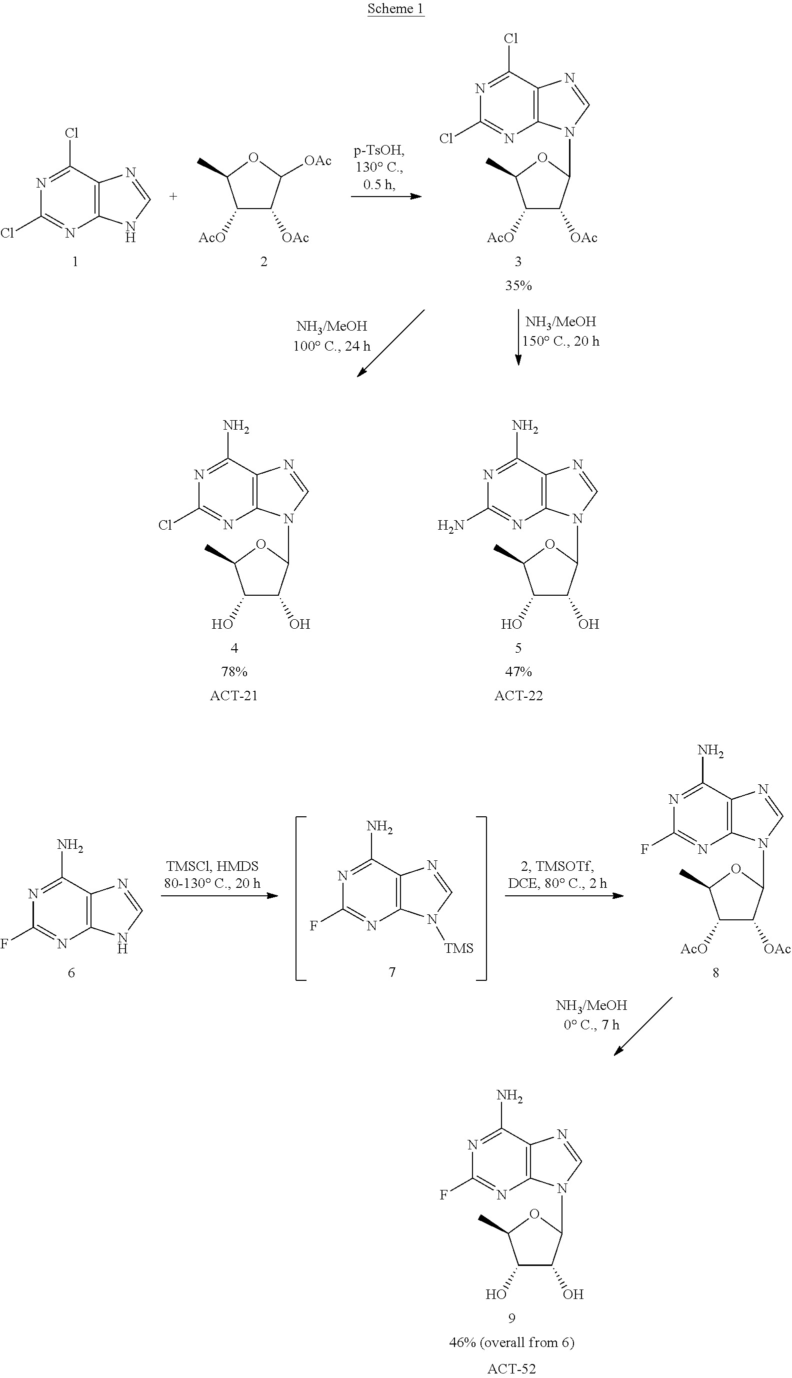

This scheme shows the synthesis of adenosines 4, 5 and 9. The known 5′-deoxyribose derivative 2³² was coupled to 2,6-dichloropurine (1) by the general method of Montgomery and Hewson³³ to afford adenosine 3. Treatment of the latter with methanol in ammonia in a pressure apparatus at 100° C. resulted in substitution of the more reactive 6-chloro substituent, along with deacetylation to produce 4, while the higher temperature of 150° C. afforded the corresponding diamino derivative 5.³⁴ 6-Amino-2-fluoropurine 6 was coupled with 2 via the trimethylsilyl derivative 7 in the presence of trimethylsilyl triflate,³⁵ followed by deacetylation of 8 to produce 9.^{33,34}.

As shown in Scheme 2 below, in order to prepare 5′-deoxy-5′-methylthio adenosines, as well as their sulfoxide and sulfone counterparts, the known ribose derivative 10³⁶ was first converted to the sulfide 12 via substitution of the mesylate 11a with sodium thiomethoxide and conversion of the acetonide and 1-methoxy moieties of 12 to the 1,2,3-diacetate 13³⁷.

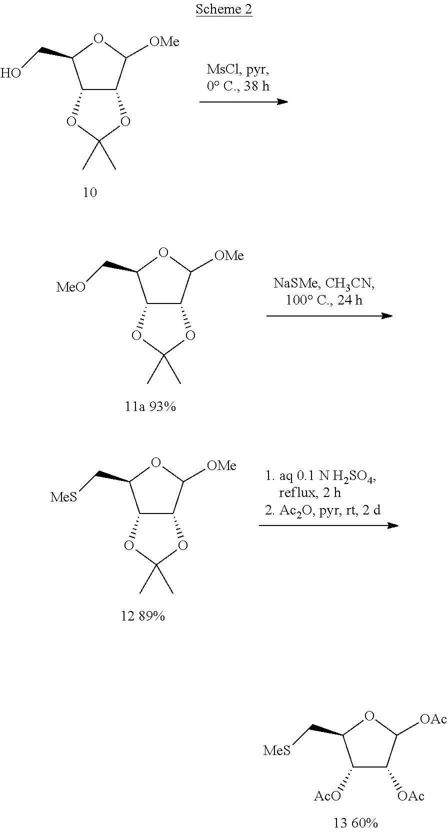 [SMILES: COC1O[C@H](CO)[C@H]2OC(C)(C)O[C@@H]12 ; COC[C@H]1OC(OC)[C@@H]2OC(C)(C)O[C@H]12 ; COC1O[C@H](CSC)[C@H]2OC(C)(C)O[C@@H]12 ; CSC[C@H]1OC(OC(C)=O)[C@H](OC(C)=O)[C@@H]1OC(C)=O]

Coupling of 13 with adenine derivatives 1, 6 and 14 proceeded via their 9-trimethylsilyl derivatives 15-17, as in the case of the preparation of 8 in Scheme 1. The 2,6-dichloro product 18³⁸ was then subjected to substitution of the 6-chloro substituent with ammonia, with concomitant deacetylation, at high temperature to produce sulfide 21,³⁸ while the 6-amino-2-fluoro analogue 19 furnished the corresponding diol 22³⁹ at 0° C. Sulfides 19 and 20 were then oxidized to either their sulfoxide or sulfone counterparts with MCPBA at either −78° C. or at reflux in dichloromethane, thereby affording 23 and 24, or 27 and 28, respectively. Deacetylation in the usual manner provided the free diols 25 and 26, or 29 and 30, respectively, as shown in Scheme 3 below. The sulfoxides 23-26 were obtained as inseparable mixtures of diastereomers.

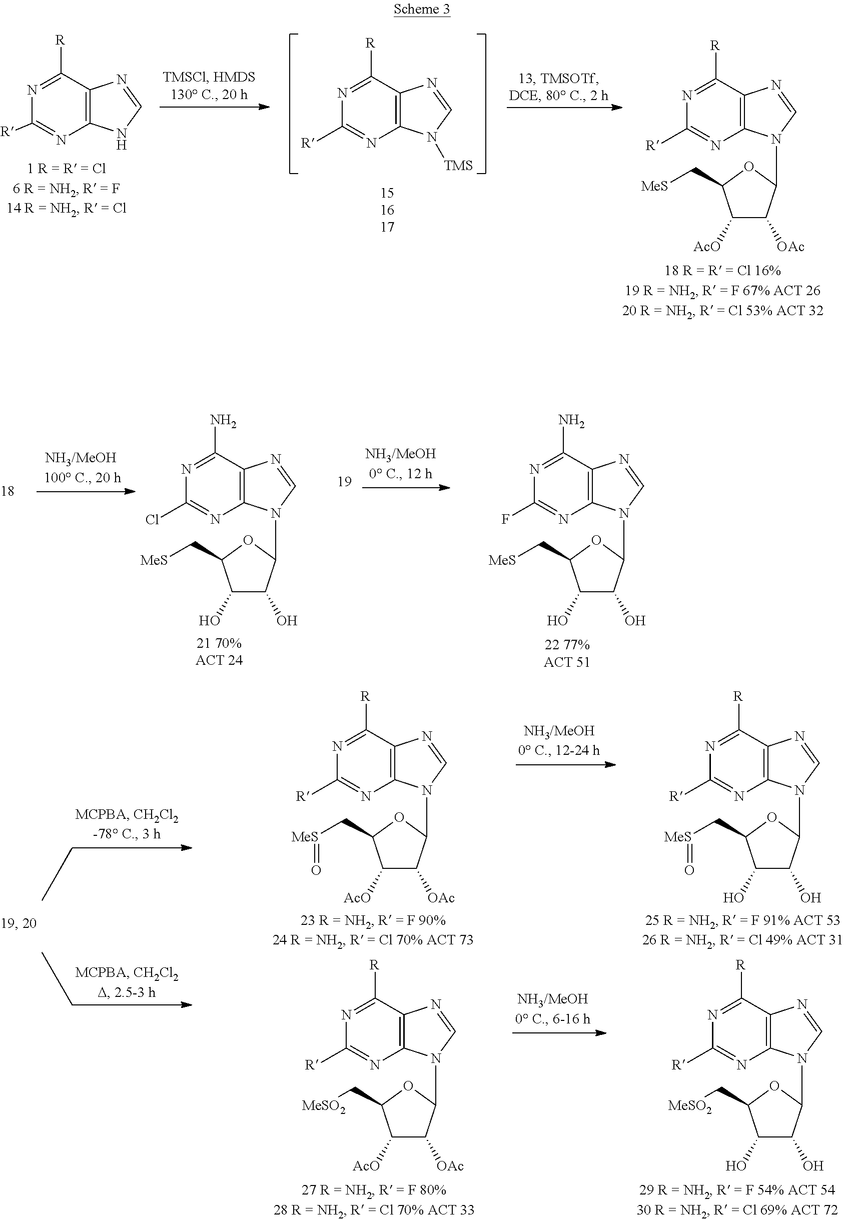

The preparation of several products containing nitrogen or iodine functionalities at the 5′-position was also carried out. Thus, mesylate 11a afforded the 5′-azido derivative 31 by treatment with sodium azide, followed by conversion to the 1,2,3-triacetate 32.^{37b,40} The latter was reduced to the corresponding 5′-acetamido-5′-deoxyribose 33 with thioacetic acid by the method of Fairbanks et al.,⁴¹ while the reaction of 11b with potassium phthalimide, followed by the usual acetylation protocol provided 35. the 5′-iodoribose derivative37^{37b} was obtained from the 1,2,3-triacetate 36, while the corresponding methoxy analogue 39³⁴ was obtained from hydrolysis and acetylation of 38 (Scheme 4). Coupling of the 5′-modified ribose derivatives 32, 33, 35 and 37 with 2-fluoro- and 2-chloroadenosine (6 and 14, respectively) via 9-N-silylation of the latter and treatment with trimethylsilyl triflate in the usual manner³⁵ then provided the 2,3-diacetates 40-47, respectively, as well as the corresponding diols 48-51 after deacetylation (Scheme 5).

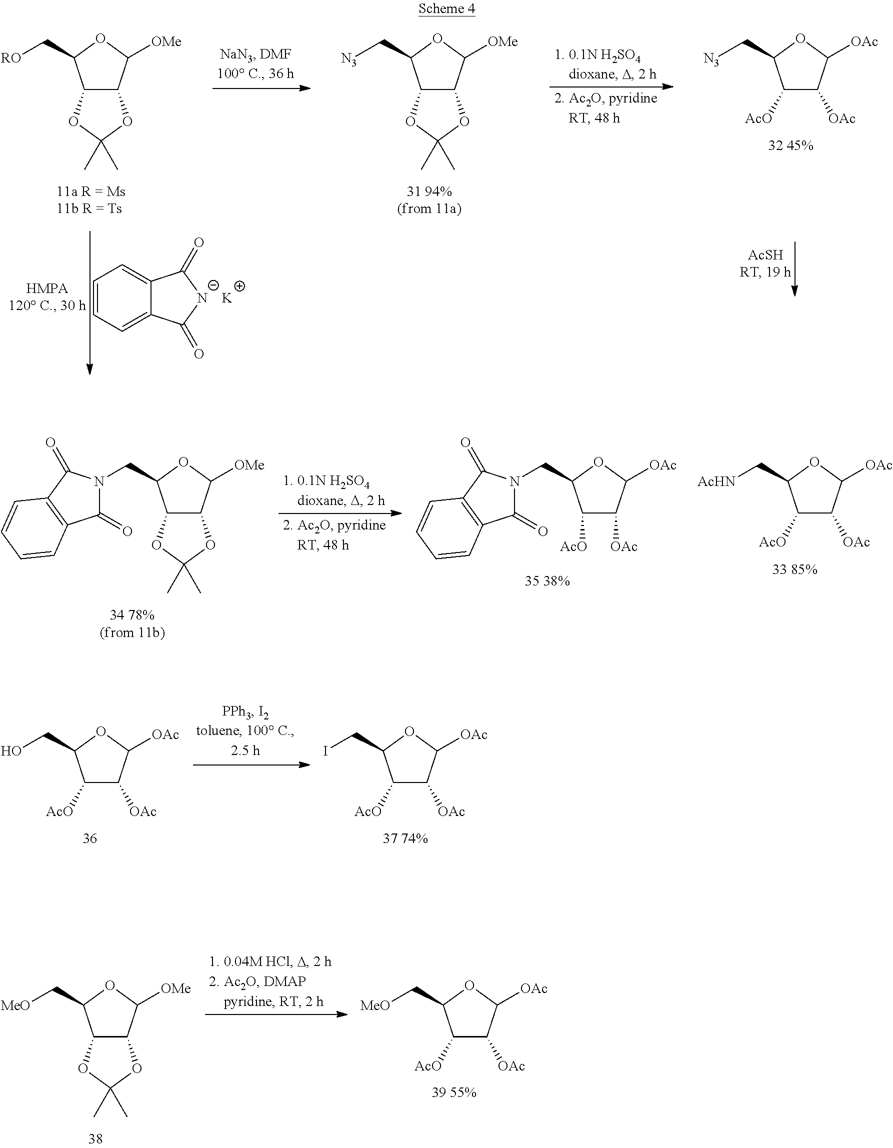

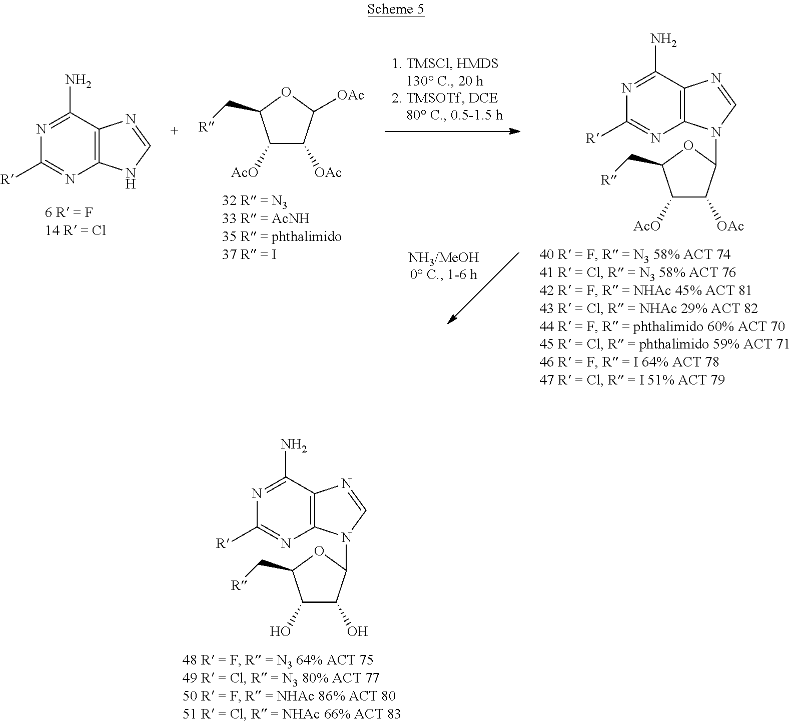

Scheme 6 shows the synthesis of 5′-Deoxy and 5′deoxy-5′-(methylthio) purines modified at the 2- or 6-position. 6-Chloropurine (52) was also coupled with ribose derivatives 2 and 13 to produce 2,3-diacetates 53 and 54, respectively, which underwent substitution at the 6-position with methylamine, along with deacetylation, to afford adenosine analogues 55 and 56.⁴²

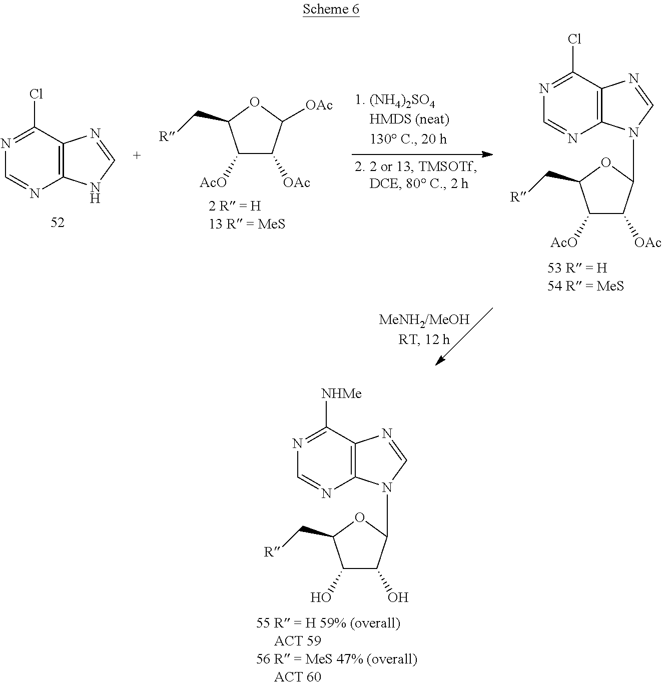

In addition to the 2-chloro and 2-fluoro derivatives shown in Schemes 1, 3 and 5, several compounds with other substituents at the 2-position were prepared in both the 5′-deoxy- and 5′-deoxy-5′-methylthioribose series. 2-Chloroadenosines 4 and 21 were converted into 57 and 58,⁴³ respectively, by substitution with hydrazine hydrate. Subsequent selective diazotization afforded the corresponding products 59 and 60, obtained as mixtures of azide and tetrazole tautomers⁴⁴ (a and b, respectively; Scheme 7).

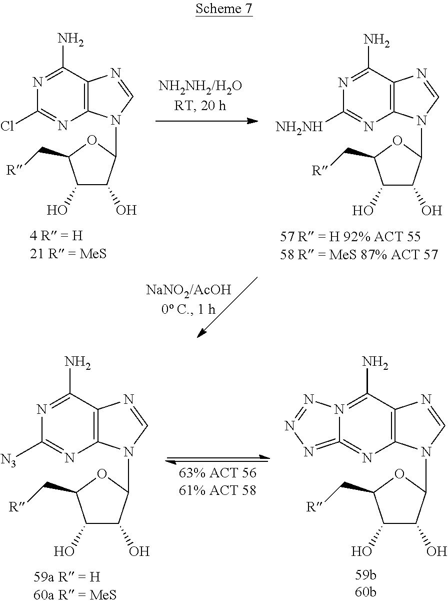

In order to introduce iodo and cyano substituents into the 2-position, 2-amino-6-chloro purine (61) was condensed with the triacetoxyribose derivatives 2 and 13. The resulting products 62 and 63 were then diazotized and converted into the corresponding 2-iodo products 64 and 65, respectively, by the general procedure of van Tilburg et al.⁴³ Substitution of the 6-chloride and simultaneous deacetylation were effected with either methanolic ammonia at 60° C., or with methylamine at room temperature in both the 5′-deoxy and the 5′-deoxy-5′-methylthio series to furnish products 66-69.⁴⁵ Further conversion of the latter four 2-iodo derivatives to the corresponding 2-cyano analogues 70-73 was achieved by a variation of the Stille reaction. Interestingly, the treatment of the 6-chloro-2-iodo compound 64 with methanolic ammonia at room temperature gave predominantly the corresponding 6-methoxy derivative 74 instead of the expected amine 66 (Scheme 8).

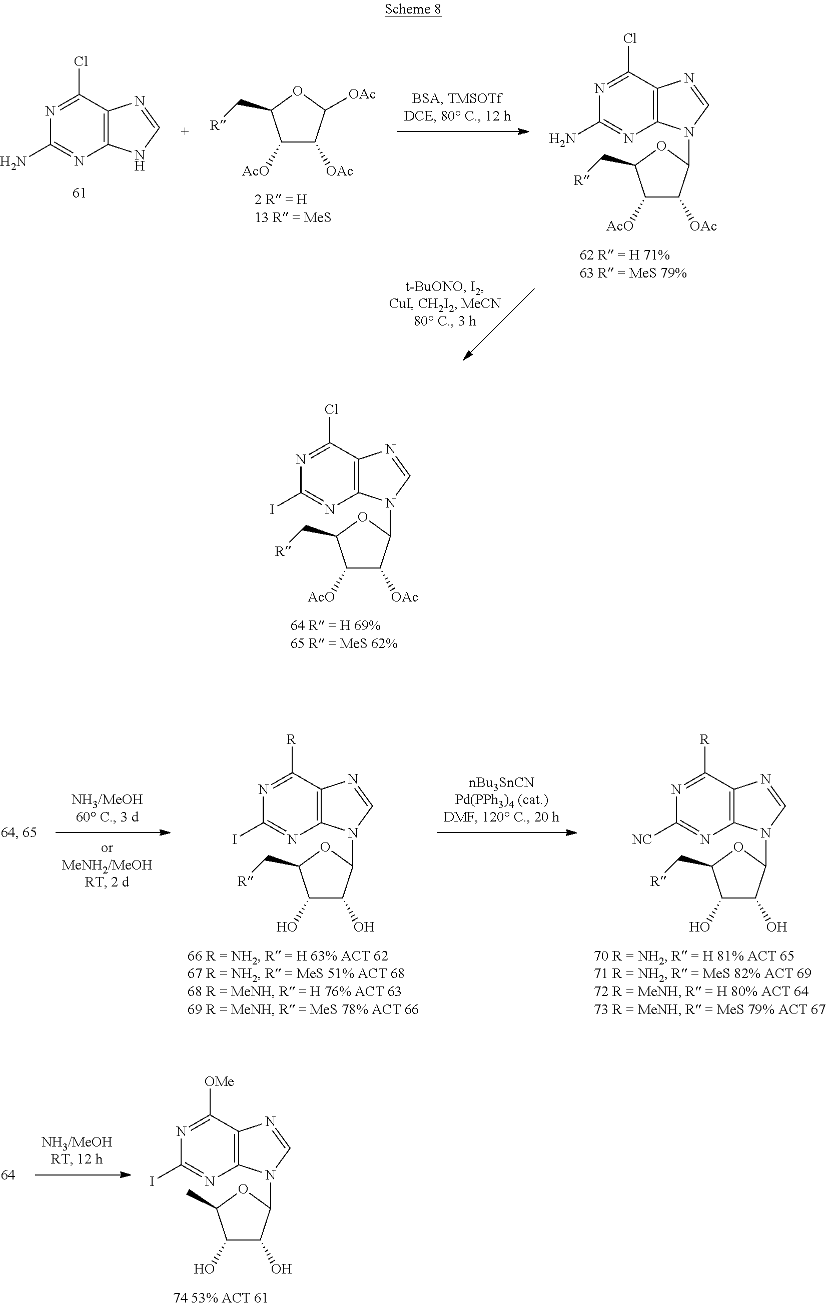

Scheme 9 shows the synthesis of 5′-deoxy- and 5′-deoxy-5′-(methylthio) purines modified at both the 2- and 6-positions. Compound 62, prepared as shown in Scheme 8, was diazotized in the presence of HF-pyridine to produce the 2-fluoro analogue 75. Substitution with methylamine at room temperature occurred at both the 2- and 6-positions, along with deacetylation, thus affording 76. When the reaction was performed at −10° C., preferential substitution of the fluoride moiety at C-2 occurred (Scheme 9), in contrast to the typically greater reactivity observed at C-6, as in the case of the 2,6-dichloro derivative 3 in Scheme 1. The treatment of 62 and 63 with methylamine in methanol produced the 6-methylamino products 78 and 79, respectively. When the latter were diazotized in the presence of HF-pyridine under various conditions, mixtures of products were obtained. However, by employing brief reaction times at −30° C., it was possible to isolate modest yields of the 2-fluoro derivative 80 and the N-nitroso compound 81 in the 5′-deoxy- and 5′-deoxy-5′-methylthio series, respectively.⁴⁶

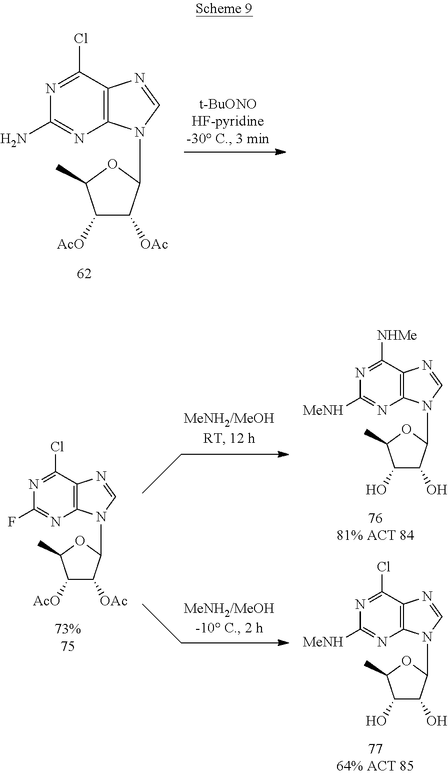

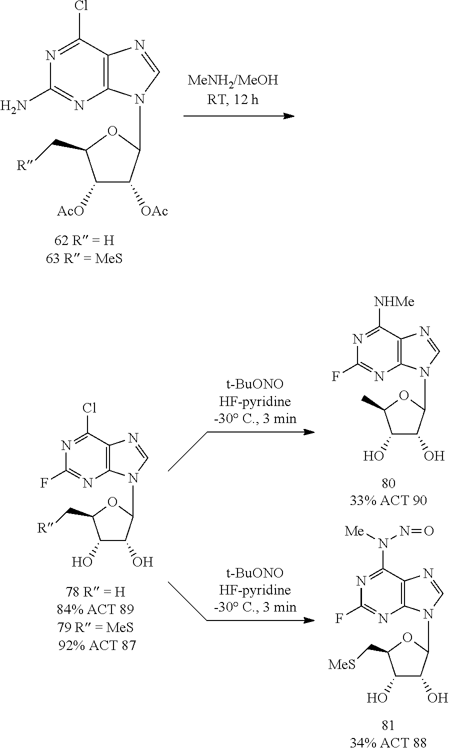

Finally, the amidine 82 was obtained by the treatment of the 6-amino group of 22 with the dimethylacetal of DMF (Scheme 10).

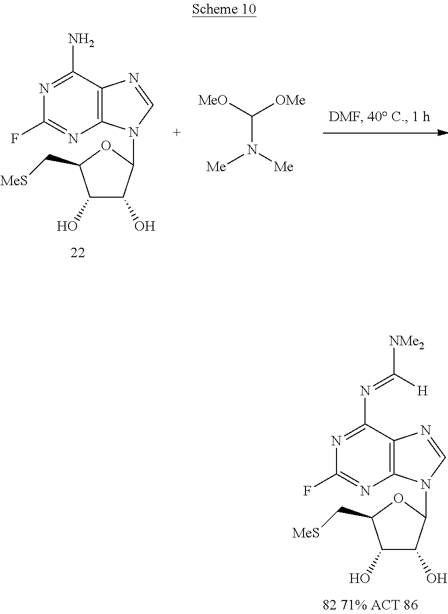 [SMILES: CSC[C@H]1OC(n2cnc3c(N)nc(F)nc32)[C@H](O)[C@@H]1O ; COC(OC)N(C)C ; CSC[C@H]1OC(n2cnc3c(/N=C/N(C)C)nc(F)nc32)[C@H](O)[C@@H]1O]

The synthesis of deaza- and deoxyguanosine derivatives is shown in Scheme 11. The 6-chloro- and 6-thio analogues 83 and 84 were obtained by literature methods.⁴⁷ An attempt to prepare the 6-seleno analogue of 6-thio-7-deazaguanosine 84 from the reaction of the corresponding chloride 83 with selenourea produced the corresponding diselenide 85, presumably via aerial oxidation of an initially formed tautomeric selenocarbonyl-selenol monomer (Scheme 11).

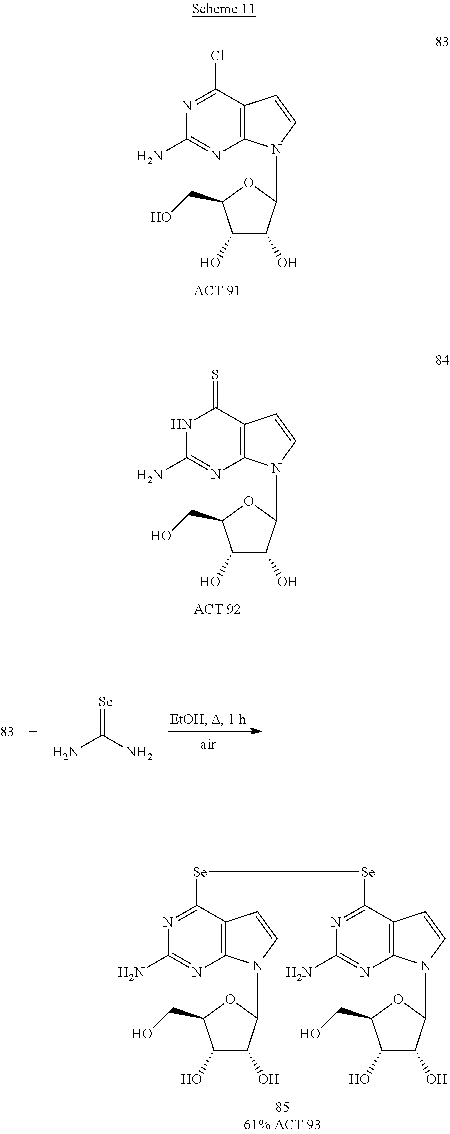

### 2-Chloro-5′-deoxyadenosine (4).³⁴

9-(2,3-Di-O-acetyl-5-deoxy-D-ribofuranosyl)-2,6-dichloropurine (3) was prepared from 2,6-dichloropurine (1) and the 5-deoxyribose derivative 2 by the general method of Montgomery and Hewson.³³ A solution of 3 (49 mg, 0.13 mmol) in methanol (17 mL) was saturated with ammonia at 0° C. for 20 min. The mixture was then stirred in a Paar apparatus at 100° C. for 24 h and cooled to room temperature. After the removal of the solvent, the resulting residue was purified by flash chromatography (dichloromethane-methanol 9:1) to provide (28 mg, 78%) of 4 as a pale yellow solid; mp 218-219° C.; IR (KBr) 3318, 3173, 1653 cm⁻¹; ¹H NMR (300 MHz, DMSO-d₆) δ 8.34 (s, 1H), 7.82 (broad s, 2H), 5.76 (d, J=5.1 Hz, 1H), 5.45 (d, J=6.0 Hz, 1H), 5.18 (d, J=5.1 Hz, 1H), 4.59 (q, J=5.2 Hz, 1H), 4.01-3.90 (m, 2H), 1.30 (d, J=6.0 Hz, 3H); ¹³C NMR (75 MHz, DMSO-d₆) δ 156.8, 153.1, 150.4, 140.3, 118.2, 87.6, 80.1, 74.5, 73.0, 18.9; MS (EI) m/z (%) 285 (M⁺, 5), 198 (82), 169 (100), 134 (70); HRMS (EI) calcd for C₁₀H₁₂³⁵ClN₅O₃(M⁺): 285.0629, found: 285.0623.

### 2-Amino-5′-deoxyadenosine (5).³⁴

A solution of 3 (160 mg, 0.41 mmol) in methanol (19 mL) was saturated with ammonia at 0° C. for 20 min. The reaction mixture was stirred in a sealed vessel at 100° C. for 4 h, then at 150° C. for 20 h and cooled to room temperature. After removal of the solvent in vacuo, the resulting residue was purified by flash chromatography (ethyl acetate-methanol, 85:15) to yield 5 (51 mg, 47%) as a pale yellow solid: mp 135-138° C.; IR (KBr) 3339, 3195, 1655, 1632 cm⁻¹; ¹H NMR (300 MHz, DMSO-d₆) δ 7.88 (s, 1H), 6.70 (broad s, 2H), 5.80 (broad s, 2H), 5.67 (d, J=5.1 Hz, 1H), 5.39 (d, J=5.1 Hz, 1H), 5.07 (s, 1H), 4.52 (d, J=4.6 Hz, 1H), 3.90 (t, J=5.1 Hz, 1H), 1.28 (d, J=5.6 Hz, 3H); ¹³C NMR (75 MHz, DMSO-d₆) δ 160.2, 156.1, 151.9, 135.9, 113.3, 86.8, 79.3, 74.6, 72.9, 19.0; MS (EI) m/z (%) 266 (M⁺, 20), 179 (20), 150 (100); HRMS (EI) calcd for C₁₀H₁₄N₆O₃ (M⁺): 266.1127, found: 266.1117.

### 5′-Deoxy-2-fluoroadenosine (9).^{34,63}

A stirred suspension of 2-fluoroadenine (6) (50 mg, 0.33 mmol) in HMDS (7.3 mL) was heated at 80° C. in a Schlenk tube. Chlorotrimethylsilane (0.026 mL, 0.21 mmol) was added dropwise and the reaction mixture was stirred at 130° C. for 20 h. After the volatile components were removed in vacuo, the resulting silylated base 7 and 5-deoxyribose derivative 2 (85 mg, 0.33 mmol) were dissolved in dry 1,2-dichloroethane (5 mL). The mixture was preheated to 80° C., trimethylsilyl triflate (24 μL, 0.13 mmol) was added dropwise and the mixture was stirred at 80° C. for 2 h. It was cooled to room temperature, dichloromethane was added, the organic layer was washed with aqueous saturated NaHCO₃ solution, water, brine and dried over anhydrous MgSO₄. Evaporation of the solvent afforded crude 8, which was used in the following step without further purification.

The crude 8 was dissolved in methanol (20 mL), and saturated with ammonia at 0° C. for 20 min. The reaction mixture was stirred at 0° C. for a further 7 h. The solvent was removed under reduced pressure, the resulting residue was purified by flash chromatography (ethyl acetate-methanol, 97:3) to afford 9 (40 mg, 46% overall) as an off-white solid; mp 244-245° C. (lit. mp⁶³ 256-258° C.); IR (KBr) 3302, 3158, 1686 cm⁻¹; ¹H NMR (300 MHz, DMSO-d₆) δ 8.30 (s, 1H), 7.84 (s, 2H), 5.73 (d, J=5.1 Hz, 1H), 5.46 (d, J=5.6 Hz, 1H), 5.18 (d, J=3.6 Hz, 1H), 4.59 (d, J=5.1 Hz, 1H), 4.00-3.93 (m, 2H), 1.29 (d, J=4.6 Hz, 3H); ¹³C NMR (75 MHz, DMSO-d6) δ 158.4 (d, J=205.0 Hz), 157.6 (d, J=21.3 Hz), 150.6 (d, J=20.6 Hz), 140.2, 117.6, 87.8, 79.9, 74.5, 73.0, 18.9; MS (EI) m/z (%) 269 (10), 182 (80), 153 (100; HRMS (EI) calcd for C₁₀H₁₂FN₅O₃(M⁺): 269.0924; found: 269.0913.

### 9-[2,3-Di-O-acetyl-5-deoxy-5-(methylthio)-D-ribofuranosyl]-2,6-dichloropurine (18).³⁸

1,2,3-Tri-O-acetyl-5-deoxy-5-(methylthio)-D-ribofuranoside (13) was prepared by literature methods^{36,37} and coupled to 2,6-dichloropurine (1) with trimethylsilyl triflate as in the procedure for the preparation of 8. The resulting product 18 was obtained in 16% yield and gave the following NMR spectra: ¹H NMR (300 MHz, CDCl₃) δ 8.37 (s, 1H), 6.67 (d, J=5.6 Hz, 1H), 5.82 (t, J=5.6 Hz, 1H), 5.50 (t, J=4.8 Hz, 1H), 4.73-4.67 (m, 1H), 2.91-2.82 (m, 2H), 2.22 (s, 3H), 2.07 (s, 3H), 1.88 (s, 3H); ¹³C NMR (75 MHz, CDCl₃) δ169.4, 168.7, 153.6, 153.1, 152.1, 144.8, 130.7, 84.0, 83.4, 72.8, 70.6, 36.9, 20.8, 20.4, 17.6.

### 2′,3′-Di-O-Acetyl-5′-deoxy-2-fluoro-5′-(methylthio)adenosine (19)

The product 19 was obtained in 67% yield from 2-fluoroadenine (6) and ribose derivative 13 by the same method used in the preparation of 8: white solid; mp 157-158° C. (dec); IR (KBr) 3321, 3171, 1750, 1744, 1676, 1616 cm⁻¹; ¹H NMR (300 MHz, CDCl₃) δ 8.00 (s, 1H), 6.75 (broad s, 2H), 6.09 (d, J=5.8 Hz, 1H), 5.84 (t, J=5.8 Hz, 1H), 5.52 (t, J=4.9 Hz, 1H), 4.38 (q, J=4.9 Hz, 1H), 3.00-2.88 (m, 2H), 2.14 (s, 3H), 2.11 (s, 3H), 2.03 (s, 3H); ¹³C NMR (75 MHz, DMSO-d₆) δ 169.7, 169.5, 159.2 (d, J=211.3 Hz), 157.4 (d, J=20.1 Hz), 151.1 (d, J=19.8 Hz), 139.0, 118.1, 85.7, 82.4, 73.0, 72.5, 36.5, 20.6, 20.4, 17.0; MS (EI) m/z (%) 399 (M⁺, 2), 196 (15), 182 (22), 154 (60), 139 (100); HRMS (EI) calcd for C₁₅H₁₈N₅O₅FS (M⁺): 399.1013; found: 399.0995.

### 2′,3′-Di-O-acetyl-2-chloro-5′-deoxy-5′-(methylthio)adenosine (20)

The product 20 was obtained in 53% yield from 2-chloroadenine (14) and ribose derivative 13 by the same method used in the preparation of 8: white solid; mp 68-71° C.; IR (KBr) 3321, 3176, 1751, 1653 cm⁻¹; ¹H NMR (300 MHz, CDCl₃-CD₃OD, 99:1) δ 8.02 (s, 1H), 6.76 (broad s, 2H), 6.13 (d, J=5.7 Hz, 1H), 5.83 (t, J=5.7 Hz, 1H), 5.52 (t, J=4.4 Hz, 1H), 4.44-4.34 (m, 1H), 2.98-2.92 (m, 2H), 2.13 (s, 3H), 2.11 (s, 3H), 2.03 (s, 3H); ¹³C NMR (75 MHz, CDCl₃-CD₃OD, 99:1) δ 169.9, 169.6, 156.7, 154.5, 150.9, 139.2, 118.9, 85.8, 82.7, 73.3, 72.7, 36.6, 20.8, 20.6, 17.2; MS (ESI) m/z (%) 416 ([M+H]⁺, 100); HRMS (ESI) calcd for C₁₅H₁₉³⁵ClN₅O₅S N₅O₅FS (M+H)⁺: 416.0795; found: 416.0797.

### 2-Chloro-5′-deoxy-5′-(methylthio)adenosine (21).³⁸

The product 21 was obtained in 70% yield from compound 18 by the same method used in the preparation of 4 from 3: white solid; mp >350° C. (dec); IR (KBr) 3414, 3333, 3277, 3223, 1654 cm⁻¹; ¹H NMR (300 MHz, DMSO-d₆) δ 8.28 (s, 1H), 7.78 (broad s, 2H), 6.21 (d, J=5.1 Hz, 1H), 5.59 (d, J=5.1 Hz, 1H), 5.47 (d, J=5.6 Hz, 1H), 4.36 (q, J=5.1 Hz, 1H), 4.24 (q, J=5.6 Hz, 1H), 4.14-4.08 (m, 1H), 2.78 (dd, J=14.1, 4.9 Hz, 1H), 2.66 (dd, J=14.1, 6.5 Hz, 1H), 2.12 (s, 3H); ¹³C NMR (75 MHz, DMSO-d6) δ 156.7, 152.9, 150.6, 141.7, 117.0, 83.3, 83.0, 73.1, 70.3, 36.1, 15.8; MS (EI) m/z (%) 331 (M⁺, 2), 198 (68), 170 (100); HRMS (EI) calcd for C₁₁H₁₄³⁵ClN₅O₃S (M⁺): 331.0506; found: 331.0505.

### 5′-Deoxy-2-fluoro-5′-(methylthio)adenosine (22).³⁹

The product 22 was obtained in 77% yield from the fluoroadenosine derivative 19 by the same method used in the preparation of 9 from 8: white solid; mp 219-220° C. (lit.³⁹ mp 213° C.); IR (KBr) 3298, 3153, 1676, 1616 cm⁻¹; ¹H NMR (300 MHz, DMSO-d6) δ 8.34 (s, 1H), 7.87 (broad s, 2H), 5.79 (d, J=5.9 Hz, 1H), 5.52 (d, J=6.1 Hz, 1H), 5.35 (d, J=5.0 Hz, 1H), 4.66 (q, J=5.6 Hz, 1H), 4.11 (q, J=4.5 Hz, 1H), 4.05-4.00 (m, 1H), 2.87 (dd, J=6.0, 13.9 Hz, 1H), 2.77 (dd, J=6.9, 14.3 Hz, 1H), 2.06 (s, 3H); ¹³C NMR (75 MHz, DMSO-d6) b 158.6 (d, J=203.7 Hz), 157.7 (d, J=21.2 Hz), 150.7 (d, J=20.5 Hz), 140.2, 117.5 (d, J=4.2 Hz), 87.3, 83.8, 72.6, 72.5, 36.0, 15.5; MS (EI) m/z (%) 315 (M⁺, 1), 212 (44), 182 (46), 154 (100); HRMS (EI) calcd for C₁₁H₁₄FN₅O₃S (M⁺): 315.0801, found: 315.0809.

### 2′,3′-Di-O-Acetyl-5′-deoxy-2-fluoro-5′-(methylthio)adenosine S-oxide (23)

A solution of MCPBA (24 mg, 77% purity, 0.11 mmol) in dichloromethane (3.5 mL) was added dropwise over 10 min to sulfide 19 (42 mg, 0.11 mmol) in dichloromethane (5 mL) at −78° C. The reaction mixture was stirred at −78° C. for a further 3 h and was then poured into aqueous saturated NaHCO₃ solution. The aqueous layer was extracted with chloroform, the combined organic layers were washed with brine, dried over anhydrous MgSO₄ and evaporated under reduced pressure. The residue was purified by flash chromatography (dichloromethane-methanol, 95:5) to afford 39 mg (90%) of the sulfoxide 23 as a mixture of diastereomers: white solid; mp 84-98° C. (dec); IR (KBr) 3330, 3188, 1752, 1653, 1040 cm⁻¹; ¹H NMR (300 MHz, CDCl₃) δ 7.98 (s, 1H), 6.60 (broad s, 2H), 6.06-5.90 (m, 2H), 5.77-5.71 (m, 1H), 4.82-4.74 (m, 1H), 3.73-2.93 (m, 2H), 2.64 (s, 3H), 2.13 (s, 3H), 2.06 (s, 3H); distinct peaks from the minor diastereomer were observed at δ 7.85 (s, 1H) and 2.62 (s 3H) ppm; dr=3:2; ¹³C NMR (75 MHz, CDCl₃) both diastereomers: δ 169.9, 169.8, 169.7, 159.3 (d, J=211.9 Hz), 159.2 (d, J=206.8 Hz), 157.7 (d, J=20.6 Hz), 150.8 (d, J=20.0 Hz), 140.4, 139.9, 118.8, 118.5, 87.9, 87.3, 73.3, 73.1, 72.9, 57.8, 53.9, 39.7, 38.9, 20.7, 20.6; MS (EI) m/z (%) 415 (M+, 0.5), 400 (68), 139 (100); HRMS (EI) calcd for C₁₅H₁₈FN₅O₆S (M⁺): 415.0962; found: 415.0973.

### 2′,3′-Di-O-Acetyl-2-chloro-5′-deoxy-5′-(methylthio)adenosine S-oxide (24)

The product 24 was obtained from sulfide 20 in 70% yield by the same method used in the preparation of 23 from 19: white solid; mp 82-95° C. (dec); IR (KBr) 3327, 3176, 1750, 1652, 1041 cm⁻¹; ¹H NMR (300 MHz, CDCl₃) δ 7.94 (s, 1H), 6.71 (broad s, 2H), 6.115.87 (m, 2H), 5.81-5.67 (m, 1H), 4.90-4.65 (m, 1H), 3.70-3.09 (m, 2H), 2.62 (s, 3H), 2.11 (s, 3H), 2.05 (s, 3H); distinct peaks from the minor diastereomer were observed at δ 7.84 and 2.66 ppm; dr=3:2; ¹³C NMR (101 MHz, CDCl₃) both diastereomers: δ 169.9, 169.8, 156.7, 156.6, 154.4, 154.2, 150.4, 150.1, 140.7, 140.2, 119.6, 119.2, 88.0, 87.4, 77.5, 73.4, 73.3, 73.1, 73.0, 57.7, 53.9, 39.9, 39.0, 20.8, 20.7, 20.6; MS (CI) m/z (%) 432 ([M+H]⁺, 100); HRMS (CI) calcd for C₁₅H₁₉³⁵ClN₅O₆S (M+H)⁺: 432.0745; found: 432.0757.

### 5′-Deoxy-2-fluoro-5′-(methylthio)adenosine S-oxide (25)

A solution of sulfoxide 23 (32 mg, 0.077 mmol) in methanol (15 mL) was saturated with ammonia at 0° C. for 20 min. The reaction mixture was stirred at 0° C. for a further 12 h. After the removal of solvent, the resulting residue was purified by flash chromatography (ethyl acetate-methanol, 9:1) to afford 25 (23 mg, 91%) as a mixture of diastereomers: white solid; mp 232-239° C.; IR (KBr) 3315, 3160, 1675, 1669, 1028 cm⁻¹; ¹H NMR (300 MHz, DMSO-d₆) δ 8.35 (s, 1H), 7.88 (broad s, 2H), 5.84-5.81 (m, 1H), 5.62-5.59 (m, 1H), 5.49 (d, J=5.0 Hz, 1H), 4.65 (q, J=5.3 Hz, 1H), 4.26 (m, 2H), 3.28-3.04 (m, 2H), 2.57 (s, 3H); distinct peaks from the minor diastereomer were observed at δ 4.32 (m) and 2.55 (m) ppm; dr=3:2; ¹³C NMR (75 MHz, DMSO-d₆) both diastereomers: δ 158.7 (d, J=203.1 Hz), 158.5 (d, J=204.0 Hz), 157.7 (d, J=21.7 Hz), 150.6 (d, J=20.6 Hz), 150.4 (d, J=20.0 Hz), 140.5, 140.2, 117.7 (d, J=4.0 Hz), 117.6 (d, J=3.8 Hz), 88.1, 87.8, 78.1, 73.1, 72.8, 72.6, 57.5, 55.0, 42.1, 38.0; MS (EI) m/z (%) 331 (M⁺, 1), 316 (100); HRMS (EI) calcd for C₁₁H₁₄FN₅O₄S (M⁺): 331.0751; found: 331.0747.

### 2-Chloro-5′-deoxy-5′-(methylthio)adenosine S-oxide (26)

Product 26 was obtained from sulfoxide 24 in 49% yield by the same method used in the preparation of 25 from 23: white solid; mp 122-128° C.; IR (KBr) 3397, 3190, 1642, 1598 1034 cm⁻¹; ¹H NMR (300 MHz, DMSO-d₆) δ 8.35 (s, 1H), 7.83 (broad s, 2H), 5.83 (m, 1H), 5.59 (m, 1H), 5.49 (m, 1H), 4.64 (m, 1H), 4.44 (m, 1H), 4.32 (m, 1H), 3.25-3.00 (m, 2H), 2.55 (s, 3H); a distinct peak from the minor diastereomer was observed at δ 8.25 (s) ppm; dr=5:1, ¹³C NMR (75 MHz, DMSO-d₆) both diastereomers: δ 156.9, 153.1, 153.0, 150.3, 150.2, 140.8, 140.4, 118.4, 118.3, 88.1, 87.8, 78.4, 78.3, 73.2, 72.7, 70.0, 57.5, 54.9, 38.0; MS (EI) m/z (%) 347 (M⁺, 1), 169 (100); HRMS (EI) calcd for C₁₁H₁₄³⁵Cl; N₅O₄S (M⁺): 347.0455; found: 347.0467.

### 2′,3′-Di-O-Acetyl-5′-deoxy-2-fluoro-5′-(methylthio)adenosine S,S-dioxide (27)

A solution of MCPBA (51 mg, 77%, 0.23 mmol) in dichloromethane (5 mL) was added dropwise over 1.5 h to a refluxing solution of sulfide 19 (43 mg, 0.10 mmol) in dichloromethane (6 mL). The reaction mixture was refluxed for a further 1 h, cooled to room temperature and added to aqueous saturated NaHCO₃ solution. The aqueous layer was extracted with dichloromethane, the combined organic layers were washed with brine, dried over anhydrous MgSO₄ and evaporated in vacuo. The residue was purified by flash chromatography (dichloromethane-methanol, 98:2) to provide 27 (37 mg, 80%) as a white solid; mp 113-114° C. (dec); IR (KBr) 3347, 3188, 1751, 1684, 1653, 1305, 1133 cm⁻¹; ¹H NMR (300 MHz, CDCl3) δ 7.85 (s, 1H), 6.65 (broad s, 2H), 6.02 (d, J=4.2 Hz, 1H), 5.85 (t, J=4.9 Hz, 1H), 5.70 (t, J=5.6 Hz, 1H), 4.80-4.72 (m, 1H), 4.03 (dd, J=14.9, 9.8 Hz, 1H), 3.42 (d, J=14.7 Hz, 1H), 2.88 (s, 3H), 2.13 (s, 3H), 2.09 (s, 3H), ¹³C NMR (75 MHz, CDCl₃) δ 169.9, 159.2 (d, J=211.9 Hz), 157.6 (d, J=20.1 Hz), 140.0, 118.6, 88.2, 77.7, 73.0, 72.6, 57.0, 43.1, 20.7, 20.6; MS (EI) m/z (%) 431 (M+, 3), 279 (58), 139 (100); HRMS (EI) calcd for C₁₅H₁₈FN₅O₇S (M⁺): 431.0911; found: 431.0910.

### 2′,3′-Di-O-Acetyl-2-chloro-5′-deoxy-5′-(methylthio)adenosine S,S-dioxide (28)

Product 28 was obtained from sulfide 20 in 70% yield by the same method used in the preparation of 27 from 19: white solid; mp 87-90° C.; IR (KBr) 3343, 3182, 1752, 1653, 1305, 1131 cm⁻¹; ¹H NMR (300 MHz, CDCl3) δ 7.86 (s, 1H), 6.64 (broad s, 2H), 6.04 (d, J=4.4 Hz, 1H), 5.86 (t, J=5.0 Hz, 1H), 5.70 (t, J=5.4 Hz, 1H), 4.82-4.76 (m, 1H), 4.12 (dd, J=14.9, 9.9 Hz, 1H), 3.40 (d, J=14.7 Hz, 1H), 2.87 (s, 3H), 2.14 (s, 3H), 2.09 (s, 3H); ¹³C NMR (75 MHz, CDCl3) δ 169.9, 156.7, 154.5, 150.2, 140.3, 119.4, 88.2, 78.0, 73.0, 72.8, 56.9, 43.1, 20.70, 20.65; MS (CI) m/z (%) 448 ([M+H]⁺, 100), 296 (83); HRMS (CI) calcd for C₁₅H₁₉³⁵ClN₅O₇S (M+H)⁺: 448.0694; found: 448.0696.

### 5′-Deoxy-2-fluoro-5′-(methylthio)adenosine S,S-dioxide (29)

Product 29 was obtained from diacetate 27 in 54% yield by the same method used in the preparation of 25 from 23: white solid; mp >350° C. (dec); IR (KBr) 3410, 3310, 3162, 1668, 1613, 1279, 1128 cm⁻¹; ¹H NMR (300 MHz, DMSO-d₆) δ 8.39 (s, 1H), 7.90 (broad s, 2H), 5.86 (broad s, 1H), 5.73-5.53 (m, 2H), 4.63 (broad s, 1H), 4.40-4.15 (m, 2H), 3.86 (dd, J=14.1, 9.8 Hz, 1H), 3.47 (d, J=14.2 Hz, 1H), 2.86 (s, 3H); ¹³C NMR (75 MHz, DMSO-d₆) δ 158.6 (d, J=203.7 Hz), 157.7 (d, J=20.7 Hz), 150.5 (d, J=20.4 Hz), 140.4, 117.7, 88.0, 78.9, 73.0, 72.4, 56.9, 42.1; MS (EI) m/z (%) 347 (M⁺, 3), 182 (67), 153 (100); HRMS (EI) calcd for C₁₁H₁₄FN₅O₅S (M⁺): 347.0700; found: 347.0710.

### 2-Chloro-5′-deoxy-5′-(methylthio)adenosine S,S-dioxide (30)

Product 30 was obtained from diacetate 28 in 69% yield by the same method used in the preparation of 25 from 23: white solid; mp 139-141° C.; IR (KBr) 3374, 3185, 1665, 1296, 1139 cm⁻¹; 1H NMR (300 MHz, DMSO-d6) δ 8.42 (s, 1H), 7.89 (broad s, 2H), 5.89 (d, J=5.9 Hz, 1H), 5.67-5.62 (m, 2H), 4.65 (d, J=4.1 Hz, 1H), 4.37-4.30 (m, 1H), 4.19 (broad s, 1H), 3.94 dd, J=14.8, 9.8 Hz, 1H), 3.48 (d, J=14.0 Hz, 1H), 2.85 (s, 3H); ¹³C NMR (75 MHz, DMSO-d6) δ 156.9, 153.1, 150.2, 140.6, 118.4, 88.0, 79.2, 73.1, 72.4, 56.9, 42.1; MS (CI) m/z (%) 364 ([M+H]⁺, 68), 212 (61), 170 (100).

### 1,2,3-Tri-O-acetyl-5-azido-5-deoxy-D-ribofuranose (32)

A mixture of mesylate 11a⁶⁴ (4.95 g, 17.5 mmol) and sodium azide (4.56 g, 70.0 mmol) in dry DMF (96 mL) was stirred at 100° C. for 36 h. To the cooled reaction mixture was added deionized water (125 mL), and the aqueous solution was extracted with ether. The combined organic layers were washed with brine, dried over anhydrous MgSO₄ and evaporated in vacuo, affording 3.80 g, (94%) of azide 31^{65,66} as a colourless oil.

A solution of azide 31 (1.46 g, 6.37 mmol) in aqueous 0.1 N H₂SO₄ (22 mL) and dioxane (9 mL) was refluxed for 2 h. The reaction mixture was neutralized with Ba(OH)₂.H₂O powder. After removal of most of the volatile material, the residue was co-evaporated with toluene (3×5 mL), dried on a vacuum pump overnight and dissolved in anhydrous pyridine (23 mL). The solution was cooled to 0° C. and acetic anhydride (3.0 mL) was added dropwise. The reaction mixture was stirred at room temperature for 48 h. After concentration under reduced pressure, water (100 mL) was added to the residue, and the aqueous solution was extracted with chloroform. The combined organic layers were washed with water, brine, dried over anhydrous MgSO₄, and evaporated in vacuo. The resulting residue was purified by flash chromatography (hexane-ethyl acetate, 8:2) to furnish azide 32^{37b,40,66} (0.87 g, 45%) as a light yellow solid; mp 65-67° C.; IR (KBr) 2103, 1754, 1226 cm⁻¹; ¹H NMR (300 MHz, CDCl3) δ 6.14 (s, 1H), 5.41-5.33 (m, 2H), 4.33-4.27 (m, 1H), 3.63 (dd, J=13.5, 3.4 Hz, 1H), 3.25 (dd, J=13.5, 4.1 Hz, 1H), 2.10 (s, 3H), 2.09 (s, 3H), 2.04 (s, 3H); ¹³C NMR (75 MHz; CDCl3) δ 169.8, 169.5, 169.3, 98.2, 80.6, 74.5, 70.5, 51.6, 21.1, 20.65, 20.60; MS (CI) m/z (%) 319 ([M+NH₄]+, 100), 242 (80); HRMS (CI) calcd for C₁₁H₁₉N₄O₇ (M+NH₄)⁺: 319.1254; found: 319.1253.

### 1,2,3-Tri-O-acetyl-5-N-acetyl-5-amino-5-deoxy-D-ribofruranose (33)

A mixture of azide 32 (1.20 g, 3.98 mmol) in thioacetic acid (1.80 mL, 20.1 mmol) was stirred at room temperature for 19 h. The reaction mixture was co-evaporated with 1:1 toluene-ethanol (3×5 mL). The resulting residue was purified by flash chromatography (hexane-ethyl acetate, 98.5:1.5) to provide amide 33 (1.07 g, 85%) as a white solid; mp 120-121° C.; IR (KBr) 3265, 1750, 1637, 1212 cm⁻¹; ¹H NMR (300 MHz, CDCl3) δ 6.11 (s, 1H), 5.80 (broad s, 1H), 5.30 (d, J=4.8 Hz, 1H), 5.17-5.13 (m, 1H), 4.28-4.21 (m, 1H), 3.74-3.66 (m, 1H), 3.33-3.24 (m, 1H), 2.10 (s, 3H), 2.09 (s, 3H), 2.05 (s, 3H), 1.97 (s, 3H); ¹³C NMR (75 MHz; CDCl3) δ 170.3, 170.0, 169.6, 169.2, 98.4, 80.4, 74.4, 71.2, 41.7, 23.3, 21.2, 20.7; MS (CI) m/z (%) 335 ([M+H₂O]⁺, 15), 258 (100); HRMS (EI) calcd for C₁₃H₁₉NO₈ (M⁺): 317.1111; found: 317.1118.

### 1,2,3-Tri-O-acetyl-5-deoxy-5-phthalimido-D-ribofuranose (35).⁶⁷

The phthalimido derivative 34 was prepared from tosylate 11b by a variation of the method of Ohrui et al.⁶⁸ in 78% yield: mp 130-131° C. (lit.⁶⁷ mp 128-128.5° C.). The product was converted into the triacetate 35 in 38% overall yield by the same procedure used for the preparation of 32 from 31; mp 122-123° C. (lit.⁶⁷ mp 115-117° C.).

### 1,2,3-Tri-O-acetyl-5-deoxy-5-iodo-D-ribofuranose (37).^{37b}

A solution of 1,2,3-tri-O-acetyl-D-ribofuranose (36)⁶⁹ (254 mg, 0.919 mmol), iodine (350 mg, 1.38 mmol), imidazole (188 mg, 2.76 mmol) and triphenylphosphine (350 mg, 1.33 mmol) in dry toluene (6 mL) was stirred at 100° C. for 2.5 h. After removal of the solvent under reduced pressure, the residue was dissolved in dichloromethane (30 mL), the solution was washed with aqueous 10% Na₂S₂O₃, and the aqueous layer was extracted with dichloromethane. The combined organic layers were washed with brine, dried over anhydrous MgSO4 and evaporated in vacuo. The resulting residue was purified by flash chromatography (hexane-ethyl acetate, 8:2), affording 264 mg (74%) of iodide 37 as a white solid; mp 80-82° C.; IR (KBr) 1751, 1218 cm⁻¹; ¹H NMR (300 MHz, CDCl3) δ 6.13 (s, 1H), 5.35 (d, J=4.8 Hz, 1H), 5.29-5.25 (m, 1H), 4.21 (q, J=6.1 Hz, 1H), 3.32 (d, J=5.7 Hz, 2H), 2.11 (s, 3H), 2.10 (s, 3H), 2.06 (s, 3H); ¹³C NMR (75 MHz, CDCl3) δ 169.7, 169.6, 169.2, 98.1, 80.5, 74.8, 74.0, 21.3, 20.8, 20.7, 5.6; MS (EI) m/z (%) 386 (M⁺, <1), 259 (100).

### 1,2,3-Tri-O-acetyl-5-O-methyl-D-ribofuranose (39)

Compound 38 was obtained by methylation of the free 5-hydroxyl group of 10 with methyl iodide, followed by hydrolysis of the acetonide moiety and acetylation to afford 39, as reported in the literature. 34,70

### Typical Procedure for the Preparation of Adenosines 40-47.

### 2′,3′-Di-O-Acetyl-5′-azido-5′-deoxy-2-fluoroadenosine (40).⁶⁶

Trimethylsilyl chloride (0.036 mL, 0.29 mmol) was added to 2-fluoroadenine (6) (70 mg, 0.46 mmol) in HMDS (6.0 mL) in a Schlenk tube preheated at 80° C. The mixture was stirred at 80° C. for 0.5 h and then at 130° C. for 20 h. After the volatile components were removed in vacuo, the resulting silylated base and azide 32 (138 mg, 0.458 mmol) were dissolved in dry 1,2-dichloroethane (6 mL). To this solution, preheated at 80° C., was added trimethylsilyl triflate (0.012 mL, 0.066 mmol). The reaction mixture was stirred at 80° C. for 0.5 h and added to aqueous saturated NaHCO₃ solution. The aqueous layer was extracted with chloroform and the combined organic layers were washed with brine, dried over anhydrous MgSO₄ and evaporated in vacuo. The residue was purified by flash chromatography (dichloromethane-methanol, 99:1) to provide 106 mg (58%) of 40 as a white solid; mp >350° C. (dec); IR (KBr) 3349, 3176, 2110, 1751, 1683 cm⁻¹; ¹H NMR (300 MHz, CDCl3) δ 8.00 (s, 1H), 6.60 (broad s, 2H), 6.12 (d, J=6.0 Hz, 1H), 5.78 (t, J=5.8 Hz, 1H), 5.51 (t, J=4.5 Hz, 1H), 4.31 (q, J=3.6 Hz, 1H), 3.79-3.67 (m, 2H), 2.12 (s, 3H), 2.04 (s, 3H); ¹³C NMR (101 MHz, CDCl₃) δ 169.9, 169.6, 159.4 (d, J=211.5 Hz), 157.6 (d, J=20.1 Hz), 151.4 (d, J=19.6 Hz), 139.0, 118.3 (d, J=3.8 Hz), 85.7, 81.7, 73.2, 71.4, 52.1, 20.7, 20.6; MS (CI) m/z (%) 395 ([M+1]⁺, 100); HRMS (CI) calcd for C₁₄H₁₆FN₈O₅(M+H)⁺: 395.1228, found: 395.1212.

Nucleosides 41-47 were prepared similarly from 2-fluoro- or 2-chloroadenine (6 and 14, respectively) and ribose derivatives 32, 33, 35 or 37, respectively. The products had the following properties.

### 2′,3′-Di-O-Acetyl-5′-azido-2-chloro-5′-deoxyadenosine (41).⁶⁶

Yield: 58%; white solid; mp 75-77° C.; IR (KBr) 3322, 3176, 2107, 1751, 1653 cm⁻¹; ¹H NMR (300 MHz, CDCl3) δ 8.02 (s, 1H), 6.17 (d, J=6.3 Hz, 1H), 5.85 (broad s, 2H), 5.76 (t, J=6.0 Hz, 1H), 5.51 (dd, J=5.7, 3.6 Hz, 1H), 4.32 (q, J=3.8 Hz, 1H), 3.77 (dd, J=13.2, 3.8 Hz, 1H), 3.71 (dd, J=13.2, 3.7 Hz, 1H), 2.14 (s, 3H), 2.05 (s, 3H); ¹³C NMR (101 MHz, CDCl3) δ 169.9, 169.6, 156.3, 154.8, 151.3, 139.3, 119.1, 85.7, 81.9, 73.3, 71.5, 52.2, 20.8, 20.6; MS (CI) m/z (%) 411 ([M+1]⁺, 100), 385 (90; HRMS (CI) calcd for C₁₄H₁₆³⁵ClN₈O₅ (M+H)⁺: 411.0932; found: 411.0912.

### 2′,3′-Di-O-acetyl-5′-N-acetyl-5′-amino-5′-deoxy-2-fluoroadenosine (42)

Yield: 45%; white solid; mp 227-229° C.; IR (KBr) 3420, 1699, 1654 cm⁻¹; ¹H NMR (300 MHz, DMSO-d₆) δ 8.33 (s, 1H), 8.03 (t, J=5.5 Hz, 1H), 7.88 (broad s, 2H), 5.76 (d, J=6.1 Hz, 1H), 5.48 (d, J=6.1 Hz, 1H), 5.27 (d, J=4.8 Hz, 1H), 4.60 (q, J=5.7 Hz, 1H), 4.03 (q, J=4.3 Hz, 1H), 3.91 (q, J=4.3 Hz, 1H), 3.45-3.26 (m, 2H), 1.83 (s, 3H); ¹³C NMR (101 MHz, DMSO-d₆) δ 169.5, 158.5 (d, J=203.8 Hz), 157.7 (d, J=21.3 Hz), 150.6 (d, J=20.2 Hz), 140.6, 117.8 (d, J=4.1 Hz), 87.5, 83.5, 73.6, 71.1, 40.9, 22.6; MS (EI) m/z (%) 326 (M⁺, 1), 196 (46), 174 (51), 154 (100); HRMS (EI) calcd for C₁₂H₁₅FN₆O₄(M⁺): 326.1139; found: 326.1146.

### 2′,3′-Di-O-acetyl-5′-N-acetyl-5′-amino-2-chloro-5′-deoxyadenosine (43)

Yield: 29%; white solid; mp 106-108° C.; IR (KBr) 3326, 3190, 1751, 1652 cm⁻¹; ¹H NMR (400 MHz, CDCl3) δ 7.82 (s, 1H), 7.46 (broad s, 1H), 6.04 (broad s, 2H), 5.94-5.87 (m, 2H), 5.505.46 (m, 1H), 4.43-4.39 (m, 1H), 4.16 (ddd, J=14.8, 8.3, 4.2 Hz, 1H), 3.39 (dt, J=14.8, 3.2 Hz, 1H), 2.18 (s, 3H), 2.16 (s, 3H), 2.03 (s, 3H); ¹³C NMR (101 MHz, CDCl3) δ 171.4, 169.6, 169.4, 156.6, 154.5, 150.5, 140.9, 120.2, 87.4, 82.9, 72.4, 71.7, 40.7, 23.6, 20.8, 20.6; MS (EI) m/z (%) 426 (M⁺, 1), 258 (27), 170 (100); HRMS (EI) calcd for C₁₆H₁₉³⁵ClN₆O₆ (M⁺): 426.1055; found: 426.1066.

### 2′,3′-Di-O-acetyl-5′-deoxy-2-fluoro-5′-phthalimidoadenosine (44)

Yield: 60%; white solid; mp 237-239° C.; IR (KBr) 3309, 3164, 1758, 1719, 1670, 1615 cm⁻¹; ¹H NMR (400 MHz, CDCl3) δ 8.00 (s, 1H), 7.83-7.80 (m, 2H), 7.71-7.67 (m, 2H), 6.21 (broad s, 2H), 6.03 (d, J=5.2 Hz, 1H), 5.76 (t, J=5.4 Hz, 1H), 5.57 (t, J=5.2 Hz, 1H), 4.51 (q, J=5.8 Hz, 1H), 4.10 (d, J=6.2 Hz, 2H), 2.09 (s, 3H), 2.05 (s, 3H); ¹³C NMR (101 MHz, CDCl3) δ 169.8, 169.7, 168.3, 159.1 (d, J=211.9 Hz), 157.4 (d, J=20.1 Hz), 151.3 (d, J=19.3 Hz), 139.5, 134.4, 132.0, 123.7, 118.5 (d, J=4.1 Hz), 86.5, 79.7, 73.6, 71.7, 39.4, 20.7, 20.6. MS (EI) m/z (%) 498 (M⁺, 1) 346 (28), 244 (100); HRMS (EI) calcd for C₂₂H₁₉N₆O₇F (M⁺): 498.1299; found: 498.1303.

### 2′,3′-Di-O-acetyl-2-chloro-5′-deoxy-5′-phthalimidoadenosine (45)

Yield: 59%; white solid; mp 256° C.; IR (KBr) 3306, 3172, 3109, 1758, 1742, 1702, 1652 cm⁻¹; ¹H NMR (300 MHz, DMSO-d₆) δ 8.44 (s, 1H), 7.93-7.82 (m, 6H), 6.12 (d, J=5.4 Hz, 1H), 5.94 (t, J=5.6 Hz, 1H), 5.53 (t, J=5.2 Hz, 1H), 4.37 (q, J=5.5 Hz, 1H), 4.13 (dd, J=14.5, 6.6 Hz, 1H), 3.93 (dd, J=14.3, 5.5 Hz, 1H), 2.03 (s, 6H); ¹³C NMR (75 MHz, DMSO-d₆) δ 169.4, 169.2, 167.7, 156.8, 153.2, 150.0, 140.4, 134.5, 131.4, 123.1, 118.1, 85.4, 79.2, 72.0, 71.2, 38.6, 20.24, 20.19. MS (EI) m/z (%) 514 (M⁺, 1), 346 (21), 244 (100); HRMS (EI) calcd for C₂₂H₁₉³⁵ClN₆O₇(M⁺): 514.1004; found: 514.0999.

### 2′,3′-Di-O-acetyl-5′-deoxy-2-fluoro-5′-iodoadenosine (46)

Yield: 64%; white solid; mp 181-182° C.; IR (KBr) 3318, 3168, 1751, 1676 cm⁻¹; ¹H NMR (300 MHz, CDCl3) δ 8.02 (s, 1H), 6.24 (broad s, 2H), 6.11 (d, J=6.1 Hz, 1H), 5.83 (t, J=6.0 Hz, 1H), 5.46 (dd, J=5.9, 3.9 Hz, 1H), 4.24 (q, J=4.7 Hz, 1H), 3.55 (d, J=5.1 Hz, 2H), 2.13 (s, 3H), 2.04 (s, 3H); ¹³C NMR (101 MHz, CDCl3) δ 169.8, 169.5, 159.5 (d, J=211.9 Hz), 157.5 (d, J=20.0 Hz), 151.5 (d, J=21.7 Hz), 139.5, 118.5, 85.8, 82.1, 73.6, 73.2, 20.8, 20.6, 5.2; MS (EI) m/z (%) 479 (M⁺, 100), 327 (100); HRMS (EI) calcd for C₁₄H₁₅FlN₅O₅ (M⁺): 479.0102; found: 479.0100.

### 2′,3′-Di-O-acetyl-2-chloro-5′-deoxy-5′-iodoadenosine (47)

Yield: 51%; pale yellow solid; mp 93° C.; IR (KBr) 3323, 3176, 1750, 1653, cm⁻¹; ¹H NMR (400 MHz, CDCl3) δ 8.04 (s, 1H), 6.15 (d, J=6.2 Hz, 1H), 5.94 (broad s, 2H), 5.81 (t, J=6.1 Hz, 1H), 5.46 (dd, J=6.0, 3.8 Hz, 1H), 4.24 (dd, J=9.0, 4.7 Hz, 1H), 3.62-3.50 (m, 2H), 2.14 (s, 3H), 2.05 (s, 3H); ¹³C NMR (101 MHz, CDCl3) δ 169.8, 169.6, 156.3, 154.8, 151.1, 139.6, 119.1, 85.7, 82.1, 73.6, 73.2, 20.8, 20.6, 5.3; MS (EI) m/z (%) 495 (M⁺, 5), 327 (100), 225 (87); HRMS (EI) calcd for C₁₄H₁₅ClIN₅O₅(M⁺): 494.9806; found: 494.9797.

### Typical Procedure for the Preparation of Adenosines 48-51.

### 5′-Azido-5′-deoxy-2-fluoroadenosine (48).⁶⁶

A solution of diacetate (40) (50 mg, 0.13 mmol) in methanol (20 mL) was saturated with ammonia at 0° C. for 20 min. The reaction mixture was stirred at 0° C. for a further 6 h. The solvent was removed under reduced pressure and the resulting residue was purified by flash chromatography (ethyl acetate) to afford 25 mg (64%) of adenosine derivative 48 as a white solid; mp 166-167° C.; IR (KBr) 3349, 3197, 2099, 1688 cm⁻¹; ¹H NMR (300 MHz, DMSO-d₆) b 8.32 (s, 1H), 7.84 (broad s, 2H), 5.80 (d, J=5.4 Hz, 1H), 5.58 (d, J=5.7 Hz, 1H), 5.38 (d, J=5.0 Hz, 1H), 4.65 (q, J=5.2 Hz, 1H), 4.14 (q, J=4.6 Hz, 1H), 4.02 (quintet, J=3.4 Hz, 1H), 3.64 (dd, J=13.1, 6.9 Hz, 1H), 3.51 (dd, J=13.1, 3.3 Hz, 1H); ¹³C NMR (101 MHz, DMSO-d₆) δ 158.6 (d, J=203.5 Hz), 157.7 (d, J=21.5 Hz), 150.6 (d, J=20.4 Hz), 140.3 (d, J=2.0 Hz), 117.6 (d, J=4.0 Hz), 87.8, 83.1, 72.7, 70.9, 51.7; MS (CI) m/z (%) 311 ([M+H]⁺, 100); HRMS (CI) calcd for C₁₀H₁₂FN₈O₃(M+H)⁺: 311.1016; found: 311.1031.

Nucleosides 49-51 were prepared similarly from 41-43, respectively. The products had the following properties.

### 5′-Azido-2-chloro-5′-deoxyadenosine (49).⁶⁶

Yield: 80%; white solid; mp 222-223° C.; IR (KBr) 3362, 3150, 2097, 1666 cm⁻¹; ¹H NMR (300 MHz, DMSO-d₆) δ 8.39 (s, 1H), 7.85 (broad s, 2H), 5.86 (d, J=5.7 Hz, 1H), 5.60 (d, J=5.9 Hz, 1H), 5.41 (d, J=5.1 Hz, 1H), 4.69 (q, J=5.5 Hz, 1H), 4.15 (q, J=4.6 Hz, 1H), 4.05 (quintet, J=3.6 Hz, 1H), 3.71 (dd, J=13.1, 7.1 Hz, 1H), 3.53 (dd, J=13.1, 3.7 Hz, 1H); ¹³C NMR (101 MHz, DMSO-d₆) δ 156.8, 153.1, 150.4, 140.4, 118.3, 87.7, 83.3, 72.7, 70.9, 51.6; MS (CI) m/z (%) 327 ([M+H]⁺, 100); HRMS (CI) calcd for C₁₀H₁₂³⁵ClN₈O₃(M+H)⁺: 327.0721; found: 327.0722.

### 5′-N-Acetyl-5′-amino-5′-deoxy-2-fluoroadenosine (50)

Yield: 86%; white solid; mp 227-229° C.; IR (KBr) 3420, 1699, 1654 cm⁻¹; ¹H NMR (300 MHz, DMSO-d6) δ 8.33 (s, 1H), 8.03 (t, J=5.5 Hz, 1H), 7.88 (broad s, 2H), 5.76 (d, J=6.1 Hz, 1H), 5.48 (d, J=6.1 Hz, 1H), 5.27 (d, J=4.8 Hz, 1H), 4.60 (q, J=5.7 Hz, 1H), 4.03 (q, J=4.3 Hz, 1H), 3.91 (q, J=4.3 Hz, 1H), 3.45-3.26 (m, 2H), 1.83 (s, 3H); ¹³C NMR (101 MHz, DMSO-d6) 169.5, 158.5 (d, J=203.8 Hz), 157.7 (d, J=21.3 Hz), 150.6 (d, J=20.2 Hz), 140.6, 117.8 (d, J=4.1 Hz), 87.5, 83.5, 73.6, 71.1, 40.9, 22.6; MS (EI) m/z (%) 326 (M⁺, 1), 196 (46), 174 (51), 154 (100); HRMS (EI) calcd for C₁₂H₁₅FN₆O₄(M⁺): 326.1139; found: 326.1146.

### 5′-N-Acetyl-5′-amino-2-chloro-5′-deoxyadenosine (51)

Yield: 66%; pale yellow solid; mp 134-135° C.; IR (KBr) 3328, 1649 cm⁻¹; ¹H NMR (300 MHz, DMSO-d₆) δ 8.38 (s, 1H), 8.02 (t, J=5.8 Hz, 1H), 7.85 (broad s, 2H), 5.79 (d, J=6.1 Hz, 1H), 5.50 (d, J=6.0 Hz, 1H), 5.29 (d, J=4.9 Hz, 1H), 4.58 (q, J=5.7 Hz, 1H), 4.07-4.00 (m, 1H), 3.94-3.87 (m, 1H), 3.36-3.25 (m, 2H), 1.83 (s, 3H); ¹³C NMR (101 MHz, DMSO-d₆) δ 169.6, 156.9, 153.1, 150.5, 140.5, 118.4, 87.3, 83.6, 72.9, 71.2, 41.0, 22.6; MS (ESI) m/z (%) 341 ([M −H]⁻, 100).

### 5′-Deoxy-N⁶-methyladenosine (55)

A mixture of 6-chloropurine 52 (85 mg, 0.55 mmol) and (NH₄)₂SO₄ (132 mg, 1.00 mmol) in HMDS (6.0 mL) was stirred at 130° C. for 20 h until the solution became clear. Volatile components were removed in vacuo and the residue, together with the 5-deoxyribose derivative 2 (130 mg, 0.500 mmol), was dissolved in 1,2-dichloroethane (8 mL), followed by the dropwise addition of trimethylsilyl triflate (0.100 mL, 0.25 mmol) at 80° C. Stirring was continued at 80° C. for 2 h. The reaction mixture was cooled to room temperature, dichloromethane was added and the solution was washed with aqueous saturated NaHCO₃ and brine, dried over anhydrous MgSO₄ and evaporated in vacuo to afford 119 mg (67%) of 9-(2,3-di-O-acetyl-5′-deoxy-β-D-ribofuranosyl)-6-chloropurine (53), which was used directly in the next step. The product was dissolved in 2.0 M methylamine in methanol (4 mL) and stirred at room temperature for 12 h. After the reaction mixture was concentrated to dryness, the residue was purified by flash chromatography to afford 78 mg (59% overall) of the adenosine derivative 55 as a white solid; 166-167° C.; IR (KBr) 3410, 3233, 3195, 1633 cm⁻¹; ¹H NMR (400 MHz, DMSO-d6) δ 8.30 (s, 1H), 8.23 (s, 1H), 7.73 (broad s, 1H), 5.84 (d, J=4.9 Hz, 1H), 5.41 (d, J=5.8 Hz, 1H), 5.14 (d, J=5.2 Hz, 1H), 4.65 (q, J=5.1 Hz, 1H), 4.00-3.92 (m, 2H), 2.95 (broad s, 3H), 1.30 (d, J=6.1 Hz, 3H); ¹³C NMR (101 MHz, DMSO-d6) δ 155.1, 152.7, 148.4, 139.6, 119.7, 87.8, 79.7, 74.6, 73.1, 27.0, 18.9; MS (EI) m/z (%) 265 (M⁺, 27), 192 (22), 178 (88), 150 (100); HRMS (EI) calcd for C₁₁H₁₅N₅O₃ (M⁺): 265.1175; found 265.1166.

### 5′-Deoxy-N⁶-methyl-5′-(methylthio)adenosine (56).⁷¹

The product was prepared in 47% overall yield from 6-chloropurine 52 and the ribose derivative 13 via the diacetate 65 by the same procedure used for the preparation of 55: pale yellow solid; mp 172-173° C.; IR (KBr) 3390, 3319, 3257, 1629 cm⁻¹; ¹H NMR (400 MHz, DMSO-d₆) δ 8.34 (s, 1H), 8.24 (broad s, 1H), 7.74 (broad s, 1H), 5.90 (d, J=5.7 Hz, 1H), 5.49 (d, J=6.0 Hz, 1H), 5.32 (d, J=4.9 Hz, 1H), 4.75 (q, J=5.3 Hz, 1H), 4.18-4.13 (m, 1H), 4.06-4.10 (m, 1H), 2.95 (broad s, 3H), 2.88 (dd, J=14.0, 5.8 Hz 1H), 2.78 (dd, J=14.0, 6.9 Hz, 1H), 2.05 (s, 3H); ¹³C NMR (101 MHz, DMSO-d₆) δ 155.0, 152.7, 148.5, 139.6, 119.6, 87.4, 83.7, 72.7, 72.6, 36.1, 27.0, 15.6; MS (EI) m/z (%) 311 (M⁺, 7), 208 (15), 178 (100); HRMS (EI) calcd for C₁₂H₁₇N₅O₃S (M⁺): 311.1052; found: 311.1053.

### 5′-Deoxy-2-hydrazinoadenosine (57)

2-Chloro-5′-deoxyadenosine 4 (94 mg, 0.33 mmol) was added to hydrazine monohydrate (5 mL). The reaction mixture was stirred at room temperature for 20 h. After the removal of solvent, the resulting residue was washed with 2-propanol three times and dried under vacuum for 12 h to afford 86 mg (92%) of product 57 as a white solid; mp 199-200° C.; IR (KBr) 3416, 3323, 3205, 1653 cm⁻¹, ¹H NMR (400 MHz, DMSO-d₆) δ 7.92 (s, 1H), 7.30 (broad s, 1H), 6.83 (broad s, 2H), 5.73 (d, J=4.9 Hz, 1H), 5.35 (d, J=5.7 Hz, 1H), 5.09 (d, J=4.9 Hz, 1H), 4.64-4.58 (m, 1H), 4.00-3.91 (m, 4H), 1.29 (d, J=6.0 Hz, 3H); ¹³C NMR (101 MHz, DMSO-d₆) δ 162.4, 156.5, 151.8, 137.0, 114.4, 87.7, 79.9, 75.2, 73.4, 19.6; MS (EI) m/z (%) 281 (M⁺, 37), 165 (100); HRMS (EI) calcd for C₁₀H₁₅N₇O₃ (M⁺): 281.1236; found: 281.1226.

### 5′-Deoxy-2-hydrazino-5′-(methylthio)adenosine (58).⁴³

Compound 58 was prepared from the adenosine derivative 21 in 87% yield in the same manner as its analogue 57: white solid; mp 168-169° C.; IR (KBr) 3424, 3333, 3210, 1643 cm⁻¹; ¹H NMR (400 MHz, DMSO-d₆) δ 7.96 (s, 1H), 7.38 (broad s, 1H), 6.85 (broad s, 2H), 5.78 (d, J=5.9 Hz, 1H), 5.40 (d, J=4.8 Hz, 1H), 5.25 (d, J=4.2 Hz, 1H), 4.72 (d, J=4.9 Hz, 1H), 4.32 (broad s, 2H), 4.15 (m, 1H), 3.99 (m, 1H), 2.86-2.76 (m, 2H), 2.06 (s, 3H). ¹³C NMR (101 MHz, DMSO-d₆) δ 161.8, 156.0, 151.4, 136.6, 114.0, 86.9, 83.4, 72.6, 72.3, 36.2, 15.6; MS (EI) m/z (%) 327 (M⁺, 11); HRMS (EI) calcd for C₁₁H₁₇N₇O₃S (M⁺): 327.1114; found 327.1121.

### 2-Azido-5′-deoxyadenosine (59a) and its tetrazole tautomer (59b).⁶⁶

Hydrazine derivative 57 (67 mg, 0.24 mmol) was dissolved in 5% aqueous acetic acid (3 mL) and the solution was stirred for 20 min in an ice-bath. Sodium nitrite (24 mg, 0.36 mmol) was added and stirring was continued for another 1 h at 0° C. A white precipitate formed and was collected by filtration, washed with water and dried under vacuum to afford 44 mg (63%) of 59 as a white solid; mp 105-106° C.; IR (KBr) 3325, 3203, 2140, 1642 cm⁻¹; ¹H NMR (400 MHz, DMSO-d₆) azide tautomer 59a: δ 8.25 (s, 1H), 7.60 (broad s, 2H), 5.74 (d, J=5.1 Hz, 1H), 5.40 (d, J=5.8 Hz, 1H), 5.14 (d, J=5.2 Hz, 1H), 4.67-4.60 (m, 1H), 3.97-3.92 (m, 2H), 1.29 (d, J=6.2 Hz, 3H); tetrazole tautomer 59b: δ 9.42 (broad s, 2H), 8.51 (s, 1H), 5.90 (d, J=4.8 Hz, 1H), 5.48 (d, J=5.8 Hz, 1H), 5.20 (d, J=5.4 Hz, 1H), 4.67-4.60 (m, 1H), 4.04-3.97 (m, 2H), 1.33 (d, J=6.2 Hz, 3H); tautomer ratio: 2:1; 13C NMR (101 MHz, DMSO-d6) azide tautomer: δ 156.7, 155.6, 150.5, 139.9, 117.0, 87.8, 79.9, 74.6, 72.8, 19.0; tetrazole tautomer: δ 153.8, 152.2, 143.1, 141.0, 111.5, 87.7, 79.8, 74.5, 73.0, 18.9; MS (EI) m/z (%) 292 (M⁺, 56), 205 (60), 176 (51), 150 (100); HRMS (EI) calcd for C₁₀H₁₂N₈O₃ (M⁺): 292.1032; found: 292.1032.

### 2-Azido-5′-deoxy-5′-(methylthio)adenosine (60a) and its tetrazole tautomer (60b).^{38,66}

Adenosine derivative 60 was prepared in 61% yield from 58 in the same manner as its analogue 59: Pale yellow solid; mp 92-93° C.; IR (KBr) 3419, 3352, 3210; 2133, 1643 cm⁻¹; ¹H NMR (400 MHz, DMSO-d₆) azide tautomer: δ 8.29 (s, 1H), 7.62 (broad s, 2H), 5.79 (d, J=6.0 Hz, 1H), 5.48 (d, J=6.2 Hz, 1H), 5.32 (d, J=5.0 Hz, 1H), 4.75-4.68 (m, 1H), 4.20-4.00 (m, 2H), 2.94-2.75 (m, 2H), 2.06 (s, 3H); tetrazole tautomer: δ 9.43 (broad s, 2H), 8.56 (s, 1H), 5.97 (d, J=5.6 Hz, 1H), 5.55 (d, J=6.1 Hz, 1H), 5.37 (d, J=5.2 Hz, 1H), 4.75-4.68 (m, 1H), 4.20-4.00 (m, 2H), 2.94-2.75 (m, 2H), 2.08 (s, 3H); tautomer ratio: 2:1; ¹³C NMR (101 MHz, DMSO-d₆) azide tautomer: b 157.2, 156.1, 151.1, 140.4, 117.4, 154.2, 152.8, 143.5, 141.5, 111.9, 87.7, 84.2, 72.9, 36.6, 16.1; MS (EI) m/z (%) 338 (M⁺, 63), 313 (100); HRMS (EI) calcd for C₁₁H₁₄N₈O₃S (M⁺): 338.0910; found: 338.0907.

### 9-(2,3-Di-O-acetyl-5-deoxy-D-ribofuranosyl)-2-amino-6-chloropurine (62)

N,O-Bis(trimethylsilyl)acetamide (BSA) (0.248 mL, 1.02 mmol) was added to a mixture of 2-amino-6-chloropurine (61) (85 mg, 0.50 mmol) and 1,2,3-O-triacetyl-5′-deoxy-D-ribofuranose (2) (130 mg, 0.500 mmol) in 8 mL of 1,2-dichloroethane. The mixture was stirred at 80° C. for 30 min until the solution became clear. After the reaction was cooled to room temperature trimethylsilyl triflate (0.116 mL, 0.650 mmol) was added dropwise. The mixture was stirred for 12 h at 80° C., cooled to room temperature, quenched with aqueous saturated NaHCO₃ solution and extracted with dichloromethane. The combined organic layers were washed with brine, dried over anhydrous MgSO4 and evaporated in vacuo. The residue was purified by flash chromatography (ethyl acetate-hexane, 1:1) to yield 131 mg (71%) of the purine derivative 62 as a white solid; ¹H NMR (400 MHz, CDCl3) δ 7.84 (s, 1H), 5.98-5.92 (m, 1H), 5.91 (d, J=4.8 Hz, 1H), 5.43 (t, J=5.4 Hz, 1H), 5.26 (broad s, 2H), 4.31-4.22 (m, 1H), 2.12 (s, 3H), 2.08 (s, 3H), 1.44 (d, J=6.4 Hz, 3H); ¹³C NMR (101 MHz, CDCl3) δ 169.8, 169.6, 159.3, 153.3, 151.8, 141.0, 125.9, 86.6, 78.5, 74.4, 72.9, 20.7, 20.5, 18.7; MS (EI) m/z (%) 369 (M⁺, 12), 201 (100), 99 (82); HRMS (EI) m/z calcd for C₁₄H₁₆³⁵ClN₅O₅(M⁺): 369.0840; found: 369.0834.

### 9-[2,3-Di-O-acetyl-5′-deoxy-5-(methylthio)-D-ribofuranosyl]-2-amino-6-chloropurine (63).⁴³

The product was prepared in 79% yield from 2-amino-6-chloropurine (61) and the ribose derivative 13 by the same procedure used for the preparation of 62. The crude material was used directly in the next step.

### 9-(2,3-Di-O-acetyl-5′-deoxy-D-ribofuranosyl)-6-chloro-2-iodopurine (64)

The 2-amino-6-chloro derivative 62 (177 mg, 0.480 mmol) was converted to the 2-iodo derivative 64 by the general method of van Tilburg,⁴³ except that t-butyl nitrite was employed instead of the isopentyl derivative, thus affording 159 mg (69%) of 64 as an off-white powder; ¹H NMR (400 MHz, CDCl3) δ 8.13 (s, 1H), 6.08 (d, J=5.2 Hz, 1H), 5.80 (t, J=5.4 Hz, 1H), 5.35 (t, J=5.2 Hz, 1H), 4.37 (m, 1H), 2.16 (s, 3H), 2.10 (s, 3H), 1.53 (d, J=6.5 Hz, 3H); ¹³C NMR (101 MHz, CDCl3) δ 169.7, 169.5, 152.5, 151.2, 143.5, 133.0, 116.8, 87.2, 79.5, 74.5, 73.4, 20.6, 20.4, 18.8; MS (EI) m/z (%) 480 (M⁺, 4), 281 (9), 201 (100); HRMS (EI) m/z calcd for C₁₄H₁₄³⁵ClI N₄O₅(M⁺): 479.9697; found: 479.9716.

### 9-[2,3-Di-O-acetyl-5-deoxy-5-(methylthio)-D-ribofuranosyl]-6-chloro-2-iodopurine (65).⁴³

The product was prepared in 62% yield from the 2-amino-6-chloro derivative 63 by the same procedure used for the preparation of 64: pale yellow solid; ¹H NMR (400 MHz, CDCl₃) δ 8.30 (s, 1H), 6.18 (d, J=5.7, 1H), 5.86 (t, J=5.8 Hz, 1H), 5.57 (dd, J=5.8, 4.2 Hz, 1H), 4.46 (dd, J=9.5, 5.3 Hz, 1H), 3.08-2.93 (m, 2H), 2.19 (s, 3H), 2.18 (s, 3H), 2.09 (s, 3H); ¹³C NMR (101 MHz, CDCl3) δ 169.6, 169.4, 152.0, 151.0, 143.6, 132.3, 116.9, 86.5, 82.9, 73.2, 72.5, 36.5, 20.6, 20.4, 17.0; MS (EI) m/z (%) 466 (14), 281 (81), 139 (100); HRMS (CI) m/z calcd for C₁₅H₁₇N₄³⁵ClIO₅S (M+H)⁺: 526.9653; found: 526.9638.

### 5′-Deoxy-2-iodoadenosine (66)

A solution of the diacetate 64 (240 mg, 0.500 mmol) in methanol (15 mL) was saturated with ammonia 0° C. for 20 min. The reaction mixture was stirred in sealed vessel at 60° C. for 3 d, and then cooled to room temperature. After the removal of solvent under reduced pressure, the residue was purified by flash chromatography (ethyl acetate-methanol, 20:1) to yield 119 mg (63%) of product 66 as a white solid; mp 133-134° C.; IR (KBr) 3422, 3247, 3197, 1641 cm⁻¹; ¹H NMR (400 MHz, DMSO-d₆) δ 8.26 (s, 1H), 7.70 (broad s, 2H), 5.75 (d, J=5.2 Hz, 1H), 5.43 (d, J=5.9 Hz, 1H), 5.17 (d, J=5.2 Hz, 1H), 4.59 (q, J=5.3 Hz, 1H), 4.02-3.90 (m, 2H), 1.30 (d, J=6.3 Hz, 3H); ¹³C NMR (101 MHz, DMSO-d₆) δ 155.9, 149.7, 139.6, 120.9, 119.0, 87.5, 80.1, 74.5, 73.0, 18.9; MS (EI) m/z (%) 377 (M⁺, 1), 290 (100); HRMS (EI) calcd for C₁₀H₁₂IN₅O₃ (M⁺): 376.9985; found: 376.9983.

### 5′-Deoxy-2-iodo-5′-(methylthio)adenosine (67).⁴³

The product was prepared in 51% yield from the diacetate 65 by the same procedure used for the preparation of 66: white solid; mp 100-101° C. (lit.⁴³ mp 90-93° C.; IR (KBr) 3425, 3193, 1639 cm⁻¹; ¹H NMR (400 MHz, DMSO-d₆) δ 8.29 (s, 1H), 7.71 (broad s, 2H), 5.82 (d, J=6.1 Hz, 1H), 5.49 (d, J=6.1 Hz, 1H), 5.33 (d, J=4.9 Hz, 1H), 4.69 (q, J=6.0 Hz, 1H), 4.15-4.11 (m, 1H), 4.04-4.01 (m, 1H), 2.86-2.78 (m, 2H), 2.07 (s, 3H); ¹³C NMR (101 MHz, DMSO-d₆) δ 155.9, 149. 8, 139.7, 120.9, 119.1, 87.2, 84.1, 72.61, 72.57, 35.9, 15.6; MS (CI) m/z (%) 424 ([M+H]⁺, 100); HRMS (CI) calcd for C₁₃H₁₇N₆NaO₃S (M++Na)⁺: 445.9754; found: 445.9753.

### 5′-Deoxy-2-iodo-N⁶-methyladenosine (68)

A mixture of diacetate 64 (240 mg, 0.500 mmol) and 2.0 M methylamine in methanol was stirred at room temperature for 2 d. After the reaction mixture was evaporated to dryness, the residue was purified by flash chromatography (ethyl acetate) to afford 148 mg (76%) of the product 68 as a white solid; mp 204-205° C.; IR (KBr) 3332, 1619 cm⁻¹; ¹H NMR (400 MHz, DMSO-d₆) δ 8.26 (s, 1H), 8.09 (broad s, 1H), 5.77 (d, J=5.2 Hz, 1H), 5.44 (d, J=5.9 Hz, 1H), 5.17 (d, J=5.3 Hz, 1H), 4.59 (q, J=5.2 Hz, 1H), 4.01-3.91 (m, 2H), 2.90 (d, J=4.0 Hz, 3H), 1.30 (d, J=6.3 Hz, 3H); ¹³C NMR (101 MHz, DMSO-d₆) δ 154.6, 148.7, 139.4, 121.1, 119.5, 87.5, 80.1, 74.5, 73.1, 27.1, 18.9; MS (EI) m/z (%) 391 (M⁺, 35), 304 (100), 276 (88), 274 (90), 148 (82); HRMS (EI) calcd for C₁₁H₁₄IN₅O₃(M⁺): 391.0141; found: 391.0134.

### 5′-Deoxy-2-iodo-N⁶-methyl-5′-(methylthio)adenosine (69)

The product was prepared in 78% yield from the diacetate 65 by the same procedure used for the preparation of 68: white solid; mp 209-210° C.; IR 3420, 3333, 3248, 1623 cm⁻¹; ¹H NMR (400 MHz, DMSO-d₆) δ 8.29 (s, 1H), 8.12 (broad d, J=3.8 Hz, 1H), 5.81 (d, J=6.1 Hz, 1H), 5.50 (d, J=6.1 Hz, 1H), 5.35 (d, J=4.9 Hz, 1H), 4.69 (q, J=5.7 Hz, 1H), 4.10-4.03 (m, 2H), 2.90 (broad s, 3H), 2.87-2.75 (m, 2H), 2.06 (s, 3H); ¹³C NMR (101 MHz, DMSO-d₆) δ 154.6, 148.7, 139.5, 121.1, 119.6, 87.2, 84.2, 72.6 (2 signals), 35.9, 27.1, 15.5; MS(EI) m/z 437(%) (M+, 6), 334 (25), 304 (100), 276 (76); HRMS (EI) calcd for C₁₂H₁₆IN₅O₃S (M+): 437.0019; found: 437.0036.

### 2-Cyano-5′-deoxyadenosine (70)

A solution of 5′-deoxy-2-iodoadenosine (66) (113 mg, 0.300 mmol), tetrakis(triphenylphosphine)palladium(0) (50 mg, 0.043 mmol) and tri-n-butyltin cyanide (105 mg, 0.333 mmol) in DMF (6 mL) was stirred at 120° C. for 20 h under argon. The solvent was evaporated in vacuo and the residue was purified by flash chromatography (ethyl acetate-methanol, 98:2) to give 67 mg (81%) of product 70: white solid; mp 190-191° C.; IR (KBr) 3332, 3193, 2234, 1748, 1651 cm⁻¹; ¹H NMR (400 MHz, DMSO-d₆) δ 8.57 (s, 1H), 7.99 (broad s, 2H), 5.85 (d, J=5.0 Hz, 1H), 5.47 (d, J=5.7 Hz, 1H), 5.21 (d, J=5.2 Hz, 1H), 4.61 (q, J=5.2 Hz, 1H), 4.03-3.92 (m, 2H), 3.95 (dd, J=9.6, 4.7 Hz, 1H), 1.32 (d, J=6.3 Hz, 3H); ¹³C NMR (101 MHz, DMSO-d₆) δ 156.2, 148.4, 142.3, 136.8, 120.8, 116.8, 88.0, 80.2, 74.5, 73.2, 18.9; MS (CI) m/z (%) 277 ([M+H]⁺, 100), HRMS (CI) calcd for C₁₁H₁₃N₆O₃ (M+H)⁺: 277.1044; found: 277.1051.

Products 71-73 were prepared from 67-69, respectively, in the same manner as the preparation of 70 from 66.

### 2-Cyano-5′-deoxy-5′-(methylthio)adenosine (71)

Yield: 82%; off-white solid; mp 184-185° C.; IR (KBr) 3405, 3327, 3198, 2247, 1651 cm⁻¹; ¹H NMR (400 MHz, DMSO-d₆) δ 8.61 (s, 1H), 8.01 (broad s, 2H), 5.90 (d, J=5.8 Hz, 1H), 5.54 (d, J=6.1 Hz, 1H), 5.38 (d, J=5.1 Hz, 1H), 4.68 (q, J=5.7 Hz, 1H), 4.13-4.07 (m, 1H), 4.07-4.04 (m, 1H), 2.86-2.50 (m, 2H), 2.07 (s, 3H); ¹³C NMR (101 MHz, DMSO-d₆) δ 156.2, 148.5, 142.2, 136.8, 120.7, 116.8, 87.6, 84.1, 72.9, 72.5, 36.0, 15.6; MS (CI) m/z (%) 323 ([M+H]⁺ 100), HRMS (CI) calcd for C₁₂H₁₄N₆NaO₃S (M+Na)⁺: 345.07403; found: 345.07401.

### 2-Cyano-5′-deoxy-N⁶-methyladenosine (72)

Yield: 80%; off-white solid; mp 193-194° C.; IR (KBr) 3368, 2239, 1632 cm⁻¹; ¹H NMR (400 MHz, DMSO-d₆) δ 8.56 (s, 1H), 8.43 (broad d, J=4.1 Hz, 1H), 5.85 (d, J=5.0 Hz, 1H), 5.47 (d, J=5.6 Hz, 1H), 5.21 (d, J=4.6 Hz, 1H), 4.61 (q, J=4.8 Hz, 1H), 4.04-3.93 (m, 2H), 2.96 (d, J=4.4 Hz, 3H), 1.32 (d, J=6.3 Hz, 3H); ¹³C NMR (101 MHz, DMSO-d₆) δ 154.9, 147.3, 142.1, 136.8, 121.4, 117.0, 88.0, 80.2, 74.5, 73.3, 27.1, 18.9; MS (EI) m/z (%) 290 (M⁺, 8), 203 (58), 175 (100); HRMS (EI) calcd for C₁₂H₁₄N₆O₃ (M⁺): 290.1127; found: 290.1132.

### 2-Cyano-5′-deoxy-N⁶-methyl-5′-(methylthio)adenosine (73)

Yield: 79%; pale yellow solid; mp 193-194° C.; IR (KBr) 3409, 3314, 3104, 2238, 1629 cm⁻¹; ¹H NMR (400 MHz, DMSO-d₆) δ 8.60 (s, 1H), 8.46 (broad d, J=4.3 Hz, 1H), 5.90 (d, J=5.8 Hz, 1H), 5.55 (d, J=6.0 Hz, 1H), 5.38 (d, J=5.0 Hz, 1H), 4.68 (q, J=5.2 Hz, 1H), 4.14 (q, J=4.3 Hz, 1H), 4.09-4.03 (m, 1H), 2.97 (d, J=4.3 Hz, 3H), 2.89 (dd, J=14.1, 5.9 Hz, 1H), 2.81 (dd, J=14.0, 7.0 Hz, 1H), 2.06 (s, 3H); ¹³C NMR (101 MHz, DMSO-d₆) δ 154.9, 147.4, 142.1, 136.9, 121.3, 117.0, 87.6, 84.1, 72.9, 72.5, 36.0, 27.2, 15.5; MS (CI) m/z 337(%) ([M+H]⁺, 100); HRMS (CI) calcd for C₁₃H₁₇N₆O₃S (M+H)⁺: 337.1083; found: 337.1094.

### 9-(5-Deoxy-D-ribofuranosyl)-2-iodo-6-methoxypurine (74)

A solution of 9-(2,3-O-diacetyl-5′-deoxy-β-D-ribofuranosyl)-2-chloro-6-iodopurine 75 (240 mg, 0.500 mmol) in methanol (15 mL) was saturated with ammonia at 0° C. for 20 min. The reaction mixture was then stirred at room temperature for 12 h. After removal of the solvent, the residue was purified by flash chromatography to afford 104 mg (53%) of the 6-methoxy derivative 85: white solid; mp 70-71° C.; IR (KBr) 3429, 1590 cm⁻¹; ¹H NMR (400 MHz, DMSO-d₆) δ 8.52 (s, 1H), 5.86 (d, J=5.2 Hz, 1H), 5.48 (d, J=5.8 Hz, 1H), 5.22 (d, J=5.2 Hz, 1H), 4.61 (q, J=5.3 Hz, 1H), 4.07 (s, 3H), 4.04-3.93 (m, 2H), 1.32 (d, J=6.4 Hz, 3H); ¹³C NMR (101 MHz, DMSO-d₆) δ 159.4, 152.5, 142.6, 121.1, 118.4, 87.9, 80.4, 74.5, 73.1, 54.7, 18.8. MS (CI) m/z (%) 393 ([M+H]⁺, 100); HRMS (CI) calcd for C₁₁H₁₄IN₄O₄(M+H)⁺: 393.0054; found: 393.0059.

### 9-(2,3-Di-O-acetyl-5′-deoxy-D-ribofuranosyl)-6-chloro-2-fluoropurine (75)

9-(2,3-Di-O-acetyl-5-deoxy-D-ribofuranosyl)-2-amino-6-chloropurine (62) (185 mg, 0.500 mmol) was added to 4 mL of 60% HF-pyridine with stirring at −50° C. The temperature was allowed to rise to −30° C. and tert-butyl nitrite (0.100 mL, 0.841 mmol) was added. Vigorous evolution of nitrogen was observed. After 3 min the solution was poured into crushed ice-water. The aqueous layer was extracted with chloroform and the organic layer was washed with brine, dried over anhydrous MgSO4 and the solvent was removed in vacuo. The residue was purified by flash chromatography (hexane-ethyl acetate, 2:1) to afford 136 mg (73%) of 75 as a white solid; ¹H NMR (400 MHz, CDCl3) δ 8.22 (s, 1H), 6.00 (d, J=5.0 Hz, 1H), 5.74 (t, J=5.3 Hz, 1H), 5.22 (t, J=5.3 Hz, 1H), 4.36-4.20 (m, 1H), 2.05 (s, 3H), 1.98 (s, 3H), 1.42 (d, J=6.4 Hz, 3H); ¹³C NMR (101 MHz, CDCl3) δ 169.6, 169.5, 157.0 (d, J=219.3 Hz), 152.8 (d, J=17.1 Hz), 144.6 (d, J=2.9 Hz), 130.9 (d, J=4.9 Hz), 87.0, 79.0, 74.2, 73.1, 20.4, 20.2, 18.5; MS (EI) m/z (%) 373 ([M++H], 13); HRMS (EI) calcd for C₁₄H₁₄³⁵ClFN₄O₅(M⁺): 372.0637; found: 372.0623.

### 5′-Deoxy-N⁶-methyl-2-(methylamino)adenosine (76)

The treatment of 75 with methylamine, as in the preparation of 68 from 64 at room temperature for 12 h, afforded the bismethylamino derivative 76 in 81% yield: white solid; mp 166-167° C.; IR (KBr) 3371, 3316, 3113 cm⁻¹; ¹H NMR (400 MHz, DMSO-d₆) δ 7.84 (s, 1H), 7.17 (broad s, 1H), 6.31 (broad s, 1H), 5.69 (d, J=4.8 Hz, 1H), 5.34 (d, J=5.7 Hz, 1H), 5.06 (d, J=5.5 Hz, 1H), 4.66-4.64 (m, 1H), 4.00-3.93 (m, 1H), 3.93-3.88 (m, 1H), 2.89 (broad s, 3H), 2.77 (d, J=4.8 Hz, 3H), 1.29 (d, J=6.4 Hz, 3H); ¹³C NMR (101 MHz, DMSO-d₆) b 160.5, 155.7, 151.2, 136.5, 114.3, 88.1, 79.8, 75.2, 73.2, 28.8, 27.3, 19.5; MS (CI) m/z (%) 294 ([M+H]⁺ 100); HRMS (EI) calcd for C₁₂H₁₈N₆O₃ (M⁺): 294.1440; found: 294.1440.

### 6-Chloro-9-(5′-deoxy-D-ribofuranosyl)-2-(methylamino)purine (77)

The treatment of 75 with methylamine, as in the preparation of 68 from 64, at room temperature for 12 h, afforded the 6-chloro-2-methylamino product 77 in 64% yield: white solid; mp 183-184° C.; IR (KBr) 3359, 3017, 1623 cm⁻¹; ¹H NMR (400 MHz, DMSO-d₆) δ 8.31 (s, 1H), 7.47 (broad s, 1H), 5.78 (d, J=5.0 Hz, 1H), 5.43 (d, J=5.5 Hz, 1H), 5.16 (d, J=4.8 Hz, 1H), 4.68 (broad s, 1H), 4.06-3.91 (m, 2H), 2.82 (d, J=4.8 Hz, 3H), 1.30 (d, J=6.3 Hz, 3H); 13C NMR (101 MHz, DMSO-d₆) δ 159.3, 153.7, 149.6, 142.0, 123.5, 87.9, 79.9, 74.6, 72.6, 28.3, 19.0; MS (EI) m/z (%) 299 (M⁺, 52), 183 (100); HRMS (EI) calcd for C₁₁H₁₄³⁵ClN₅O₃ (M⁺): 299.0785; found: 299.0770.

### 2-Amino-5′-deoxy-N⁶-methyladenosine (78)

The treatment of 62 with methylamine, as in the preparation of 68 from 64, at room temperature for 12 h, afforded the N⁶-methylamino derivative 78 in 84% yield: white solid; mp 207-208° C.; IR (KBr) 3462, 3362, 3305, 1629 cm⁻¹; ¹H NMR (400 MHz, DMSO-d₆) δ 7.86 (s, 1H), 7.20 (broad s, 1H), 5.87 (broad s, 2H), 5.68 (d, J=5.1 Hz, 1H), 5.35 (d, J=5.8 Hz, 1H), 5.05 (d, J=5.1 Hz, 1H), 4.52 (q, J=5.1 Hz, 1H), 3.93-3.85 (m, 2H), 2.89 (broad s, 3H), 1.28 (d, J=6.1 Hz, 3H); ¹³C NMR (101 MHz, DMSO-d6) δ 160.2, 155.4, 151.0, 135.5, 113.6, 86.8, 79.3, 74.6, 72.9, 26.9, 19.0; MS (EI) m/z (%) 280 (M⁺, 5), 164 (100); HRMS (EI) calcd for C₁₁H₁₆N₆O₃ (M⁺): 280.1284; found 280.1279.

### 2-Amino-5′-deoxy-N⁶-methyl-5′-(methylthio)adenosine (79)

Product 79 was obtained from sulfide 63 as in the preparation of 68 from 64 in 92% yield: white solid; mp 206207° C.; IR (KBr) 3490, 3463, 3321, 3233 1642 cm⁻¹; ¹H NMR (400 MHz, DMSO-d₆) δ 7.89 (s, 1H), 7.19 (broad s, 1H), 5.88 (broad s, 2H), 5.73 (d, J=6.0 Hz, 1H), 5.41 (d, J=6.1 Hz, 1H), 5.21 (d, J=4.9 Hz, 1H), 4.63 (q, J=5.9 Hz, 1H), 4.11-4.04 (m, 1H), 3.993.94 (m, 1H), 2.88 (broad s, 3H), 2.84 (dd, J=13.9, 6.0 Hz, 1H), 2.75 (dd, J=13.9, 6.9 Hz, 1H), 2.06 (s, 3H); ¹³C NMR (101 MHz, DMSO-d₆) δ 160.8, 155.9, 151.5, 136.0, 114.1, 86.9, 83.9, 73.1, 72.9, 36.6, 27.4, 16.1; MS (EI) m/z (%) 326 (M⁺, 29), 193 (100); HRMS (EI) calcd for C₁₂H₁₈N₆O₃S (M⁺): 326.1161; found: 326.1149.

### 5′-Deoxy-2-fluoro-N⁶-methyladenosine (80)

A solution of 2-amino-5′-deoxy-N⁶-methyladenosine (78) (100 mg, 0.357 mmol) in 4 mL of 60% HF-pyridine was stirred at −50° C. The temperature was allowed to rise to −30° C. and tert-butyl nitrite (0.100 mL, 0.841 mmol) was added. Vigorous evolution of nitrogen was observed. Stirring was continued for 3 min and the solution was poured into crushed ice-water and extracted with chloroform. The organic layer was washed with brine, dried over anhydrous MgSO4 and evaporated in vacuo. The residue was separated by flash chromatography (ethyl acetate) to provide 34 mg (33%) of 80 as a white solid; mp 176-177° C.; IR (KBr) 3310, 3107, 1641 cm⁻¹; ¹H NMR (400 MHz, DMSO-d₆) δ 8.33 (broad s, 1H), 8.29 (s, 1H), 5.74 (d, J=5.0 Hz, 1H), 5.45 (d, J=4.4 Hz, 1H), 5.19 (broad s, 1H), 4.58 (q, J=4.3 Hz, 1H), 4.00-3.91 (m, 2H), 2.91 (d, J=4.6 Hz, 3H), 1.29 (d, J=6.2 Hz, 3H); ¹³C NMR (101 MHz, DMSO-d₆) δ 159.2 (d, J=204.0 Hz), 157.0 (d, J=21.0 Hz), 149.9 (d, J=20.5 Hz), 140.4, 118.6 (d, J=3.9 Hz), 88.3, 80.4, 75.0, 73.5, 27.6, 19.4; MS (CI) m/z (%) 284 [(M +H)⁺, 100]; HRMS (EI) calcd for C₁₁H₁₄FN₅O₃(M⁺): 283.1081; found: 283.1086. No other pure compounds could be isolated from the reaction mixture.

### 5′-Deoxy-2-fluoro-N⁶-methyl-5′-methylthio-N⁶-nitrosoadenosine (81)

Compound 79 was treated under the same reaction conditions as in the preparation of 80 from 78 to afford 34% of the N-nitroso derivative 81 as the only isolable pure product: pale yellow solid; mp 154-155° C.; IR (KBr) 3456, 3124, 1596, 1509 cm⁻¹; ¹H NMR (400 MHz, DMSO-d₆) δ 8.85 (s, 1H), 6.00 (d, J=5.6 Hz, 1H), 5.63 (d, J=5.9 Hz, 1H), 5.43 (d, J=5.2 Hz, 1H), 4.70 (q, J=5.6 Hz, 1H), 4.18-4.14 (m, 1H), 4.12-4.07 (m, 1H), 3.54 (s, 3H), 2.90 (dd, J=14.0, 5.8 Hz, 1H), 2.82 (dd, J=14.0, 6.9 Hz, 1H), 2.08 (s, 3H); ¹³C NMR (101 MHz, DMSO-d₆) δ 157.2 (d, J=210.0 Hz), 155.6 (d, J=17.5 Hz), 153.5 (d, J=17.9 Hz), 146.3 (d, J=2.5 Hz), 122.7 d, J=4.6 Hz), 88.2, 84.7, 73.4, 72.9, 36.4, 29.6, 16.1; MS (ESI) m/z (%) 359 ([M+H]⁺, 35), 330 (100); HRMS (ESI) calcd for C₁₂H₁₆FN₆O₄S (M+H)⁺: 359.0932; found: 359.0935.

### 5′-Deoxy-2-fluoro-N⁶-[(1-dimethylamino)methylidene]-5′-(methylthio)adenosine (82)

A mixture of 6-amino-5′-deoxy-2-fluoro-5′-(methylthio)adenosine (22) (157 mg, 0.500 mmol) and N,N-dimethylformamide dimethyl acetal (0.50 mL, 3.8 mmol) in 2 mL of anhydrous DMF was stirred at 40° C. under nitrogen for 1 h. The solution was evaporated under vacuum and the residue was purified by column chromatography (ethyl acetate-methanol, 20:1) to afford 131 mg (71%) of product 82: white solid; mp 65-66° C.; IR (KBr) 3395, 1637, 1583 cm⁻¹; ¹H NMR (400 MHz, DMSO-d₆) δ 8.90 (s, 1H), 8.45 (s, 1H), 5.84 (d, J=5.9 Hz, 1H), 5.52 (d, J=6.1 Hz, 1H), 5.34 (d, J=5.0 Hz, 1H), 4.69 (q, J=5.7 Hz, 1H), 4.12-4.07 (m, 1H), 4.08-4.00 (m, 1H), 3.23 (s, 3H), 3.15 (s, 3H), 2.87 (dd, J=13.9, 5.9 Hz, 1H), 2.78 (dd, J=13.9, 6.9 Hz, 1H), 2.06 (s, 3H); ¹³C NMR (101 MHz, DMSO-d₆) δ 161.6 (d, J=16.3 Hz), 159.4, 158.1 (d, J=206.0 Hz), 153.3 (d, J=19.7 Hz), 142.3, 124.5, 87.8, 84.4, 73.1, 73.0, 41.4, 36.5, 35.3, 16.0; MS (EI) m/z (%) 370 (M⁺, 10), 267 (38), 237 (100); HRMS (EI) calcd for C₁₄H₁₉FN₆O₃S (M⁺): 370.1223; found: 370.1215.

### 2-Amino-6-chloro-7-deaza-9-(D-ribofuranosyl)purine (83).⁴⁷

Compound 83 was prepared by the method of Ramasamy et al.^{47b} Off-white solid; mp 172-173° C. (lit.⁴⁷a mp 170-172° C.); IR (KBr) 3324, 3206, 1627 cm⁻¹; ¹H NMR (400 MHz, DMSO-d₆) δ 7.38 (d, J=3.8 Hz, 1H), 6.69 (broad s, 2H, partially exchanged), 6.37 (d, J=3.8 Hz, 1H), 5.99 (d, J=6.3 Hz, 1H), 4.31 (dd, J=6.2, 5.2 Hz, 1H), 4.06 (dd, J=5.0, 3.1 Hz, 1H), 3.85 (dd, J=7.3, 3.9 Hz, 1H), 3.59 (dd, J=11.9, 4.1 Hz, 1H), 3.51 (dd, J=11.9, 4.1 Hz, 1H); 13C NMR (101 MHz, DMSO-d₆) δ 159.8, 154.8, 151.6, 123.8, 109.4, 100.2, 86.5, 85.3, 74.1, 71.0, 62.0; MS (EI) m/z (%) 300 (M⁺, 25), 168 (100); HRMS (EI) calcd for C11H₁₃₃₅ClN₄₀₄ (M⁺): 300.0625; found: 300.0610.

### 2-Amino-7-deaza-9-(D-ribofuranosyl)-6-thiopurine (84).⁴⁷

Compound 84 was prepared by the method of Seela et al.⁴⁷a Pale yellow solid; mp 216-218° C., lit.⁴⁷a mp 225-228° C.; IR (KBr) 3479, 3428, 3331, 3210, 3090, 1627 cm⁻¹; ¹H NMR (400 MHz, DMSO-d₆) δ 11.76 (s, 1H), 7.14 (d, J=3.7 Hz, 1H), 6.60 (broad s, 2H), 6.41 (d, J=3.7, Hz, 1H), 5.86 (d, J=6.2, Hz, 1H), 4.23 (t, J=5.7 Hz, 1H), 4.03 (dd, J=5.0, 3.3 Hz, 1H), 3.82 (q, J=3.8 Hz, 1H), 3.57 (dd, J=11.9, 4.1 Hz, 1H), 3.50 (dd, J=11.8, 4.1 Hz, 1H); ¹³C NMR (101 MHz, DMSO-d₆) δ 176.2, 152.7, 148.2, 121.0, 113.6, 104.8, 86.4, 85.2; 74.2, 71.0, 62.0; MS (ESI) m/z (%) 299 ([M+H]⁺, 100); HRMS (ESI) calcd for C₁₁H₁₅N₄O₄S (M+H)⁺: 299.0809; found: 299.0813.

### Bis-6,6′-[7-deaza-(9-D-ribofuranosyl)purinyl] diselenide (85)

The general method of Milne and Townsend was employed.⁷² 2-Amino-6-chloro-7-deaza-9-(D-ribofuranosyl)purine (83) (105 mg, 0.350 mmol) and selenourea (48 mg, 0.39 mmol) were refluxed in absolute ethanol (6 ml) for 1 h. The yellow reaction mixture was cooled to room temperature and the product crystallized from the solution. It was washed with ethanol and dried under vacuum to provide 74 mg (61%) of diselenide 85 as a pale yellow solid: mp 134-135° C.; ¹H NMR (400 MHz, DMSO-d₆) δ 7.30 (d, J=3.7 Hz, 2H), 6.57 (d, J=3.5 Hz, 2H), 6.48 (broad s, 4H), 6.00 (d, J=6.3 Hz, 2H), 5.10 (broad s, 6H), 4.30 (t, J=5.6 Hz, 2H), 4.07-4.03 (m, 2H), 3.84 (q, J=3.4 Hz, 2H), 3.58 (dd, J=11.8, 3.8 Hz, 4H), 3.50 (dd, J=12.0, 3.9 Hz, 6H); ¹³C NMR (101 MHz, DMSO-d₆) δ 158.9, 153.1, 153.0, 123.0, 112.3, 101.3, 86.4, 85.3, 74.0, 71.1, 62.1; HRMS (ESI) calcd for C₂₂H₂₇N₈O₈⁸⁰Se₂ (M+H)⁺: 691.0282; found: 691.0309; calcd for C₂₂H₂₆N₈NaO₈Se₂ (M+Na)⁺: 713.0102; found: 713.0132.

### Example 2: Bioactivity

Each of the nucleoside analogues was examined for in vitro activity against four parasites, T.b. rhodesiene, T. cruzi, L. donovoni and P. falciparum, as well as for cytotoxicity toward the L6 rat myoblast cell line.

The results are summarized in FIG. 2. All IC₅₀ values are expressed as micromolar. Cytotoxicity (L6 rat myoblast cells); average of duplicate determinations. Trypanosoma brucei rhodesiense (STIB900); average of duplicate determinations. Selectivity index for T.b. rhodesiense (SIᵣ); expressed as the ratio [IC50 (L6/IC₅₀ (T.b. rhodesiense)]. Plasmodium falciparum (11, resistant to chloroquine); average of duplicate determinations. Selectivity index for P. falciparum (SIₚ), expressed as the ratio [IC50 (L6/IC₅₀ (P. falciparum)]. Leishmania donovani (MHOM/SD/62/IS-CL2D) axenic amastigotes; average of duplicate determinations. Selectivity index for L. donovani (SI_{L}), expressed as the ratio [IC₅₀ (L6/IC₅₀ (L. donovani)]. Trypanosoma cruzi; average of duplicate determinations. Selectivity index for T. cruzi (SIC), expressed as the ratio [IC₅₀(L6/IC₅₀(T. cruzi)]. IA. Nucleoside analogs designed to be activated via Pathway I. IB. Nucleoside analogs designed to be activated via Pathway II. Phth=phthalimido.

All in vitro assays were carried out in collaboration with the Department of Medical Parasitology and Infection Biology, at the Swiss Tropical Institute, Basel, Switzerland.

Preparation of compounds for assay. Compounds were dissolved in 100% dimethyl sulfoxide (DMSO) and diluted in the culture medium prior to the in vitro assay. The DMSO concentration never exceeded 1% in the in vitro Alamar Blue assays at the highest drug concentration.

In vitro growth inhibition assay of T.B. rhodesiense (STIB900). IC₅₀ values were determined using the Alamar blue assay and were carried out twice independently and in duplicate as described.⁵⁷

In vitro growth inhibition assay of P. falciparum (KI). The determination of IC₅₀ values against erythrocytic stages of P. falciparum was carried out in duplicate using the [³H]-hypoxanthine incorporation assay.^{58,59}

In vitro growth inhibition assay of L. Donovani. Axenic amastigotes of L. Donovaniwere adapted from promastigotes and grown in the amastigote medium previously described.⁶⁰ The tetrazolium dye-based CellTiter reagent (Promega, Madison, Wis.) was used to assess parasite growth.⁶¹

In vitro cytoxicity assay (L6 Rat Myoblast cells). IC₅₀ values were determined using the Alamar blue assay⁶² and were carried out twice independently.

### Results and Discussion

### Findings for Pathway I.

Although several nucleoside analogues gave IC₅₀s in the micromolar range, one, ACT-88 (81) stands out with an IC₅₀ against Plasmodium falciparum of 110 nM, and a selectivity index of 983 (FIG. 2). The activity of ACT-88 with P. falciparum, as a cleavable ribonucleoside, requires activation either by conversion to its corresponding base, via a hydrolase or phosphorylase prior to phosphoribosylation to the active nucleotide form. Direct phosphorylation is blocked by the presence of the 5′-methylthio group in ACT-88. The purine nucleoside phosphorylase from P. falciparum does not exhibit activity with 6-amino purine ribosides, nor does this parasite express substantial APRT activity in contrast to relatively abundant HGXPRT. An adenosine nucleoside phosphorylase has been identified in Trypanosoma bruce¹⁴⁸ and Leishmania donovan⁴⁹ with the most recent enzymological evidence for Shistosoma mansoni.^{13,50} This activity, however, is absent from Plasmodium falciparum, Giardia lambia and Entamoeba invadens.⁵ The 5′-deoxy-5′-meththioadenosine/adenosine phosphorylase present in Leishmania donovani, Trypanosoma cruzi and Trypanosoma brucei is also absent in P. falciparum. That leaves the IAG nucleoside hydrolase as the potential route of activation for the prodrug ACT-88; however, there is as no evidence for the presence of this activity in P. falciparum.

Alternatively, the complex N-methyl-N-nitroso components of the C-6 nitrogen substituent of ACT-88 may render the analog a substrate for the Pf PNP. Were this the case, the corresponding base would be activated by the abundant PfHGXPRT activity⁵³ to the active nucleotide form. Consideration of this possibility is supported by the absence of ACT-88 activity against Trypanosoma brucei, Trypanosoma cruzi and L. donovani, which do possess either or both the IAG-NH, adenosine nucleoside phosphorylase, and/or the 5′-deoxy-5′methylthioadenosine/adenosine phosphorylase,^{14,50} which appear to not have accepted the purine C-6 substituent of ACT-88.

### Findings for Pathway II.

Purine ribonucleosides having either the N-7 or N-9 of the purine ring replaced by carbon are refractory to cleavage to the corresponding base. The results for these studies are given in FIG. 2. For inosine and guanosine analogues there is no corresponding mammalian ribonucleoside kinase, whereas a corresponding guanosine kinase or nucleoside phosphotransferase has been described for protozoa. By blockage of the pathway for cleavage to the base analogue in the mammalian host, the nucleoside analogues cannot be converted by HGPRT to the corresponding nucleotide and thereby exhibit toxicity towards the host. In contrast, single step activation of the prodrug nucleoside via phosphorylation is possible in the protozoan target. Previous work for this class had shown that 9-deazainosine had a favourable ratio of dose-cell toxicity in vitro⁵⁴ and a favourable therapeutic index in L. donovani infected hamsters and squirrel monkeys.⁵⁵ ACT-91 (83) gave an IC₅₀ of 130 nM and 60 nM with T.b. rhodesiense and L. Donovani, respectively, and an IC₅₀ of 3.4 μM with T. cruzi. The selectivity indices were also favourable for each of these, with the SI being 1250, 2720, 48 with T.b. rhodesiense, L. Donovani and T. cruzi respectively.

Formycin B, the 9-deza-8-aza-inosine analog inhibits Leishmania donovani in vitro and was therapeutically active in a hamster model.²⁹ Both formycin B and 9-deaza-inosine were active against T.b gambiense and T.b. rhodesiense.³⁰ Furthermore, the phosphorylated product of formycin B inhibited the conversion of IMP to AMP by inhibition of adenylosuccinate synthase.²⁹ Cellular nucleotide analyses have shown that 9-dezainosine is converted to both the ATP and GTP analogues by L. donovani.²⁸ Studies in Leishmani provided enzymological evidence for the initial transformation of the 9-deaza analogs to the nucleotide form to be carried out by a nucleoside phosphotransferase.²⁵ Taken together, these earlier studies provide evidence for the conversion of the non-cleavable nucleoside analogues to their active nucleotides via a nucleoside phosphotransferase in both Trypanosoma and Leishmani. A unique guanosine kinase has also been described for Trichomonas vaginalis.²⁴ 9-Deaza inosine was shown to have activity against L. donovani in a squirrel monkey in vivo model.⁵⁵

### Impact of Structural Variation on Activity and Selectivity Index.

A comparison can be made of the effect of structural variation at a given position upon bioactivity for various subsets of compounds differing at only that position.

Purine Ring C-2 Substituents.

Modification at the 2-position substituent of the purine ring was considered essential as a means of preventing the deamination of the adenosine analogs to their inosine counterparts, which might thereby become toxic to the host. In general, FIG. 2 indicates that the fluorine substituent at the 2-position gave lower IC₅₀ and lower SI values than either chlorine or iodine. The halogen substituents were also in general more active than the CN, NHNH₂ or N₃ substituents. Of note, there are no comparators at C-2 for the outstanding IC₅₀ and SI for ACT-88, other than it having shown exceptional activity with F at the 2-position.

5′-Ribose Modification.

Alteration of the 5′-OH of the ribose ring was also considered essential in order to block the direct conversion of the analogues to toxic nucleotides in the host via adenosine kinase activity. Of the modifications which could not be phosphorylated, both MeS and H were superior in yielding lower IC₅₀s than other structural changes, with MeS generally exhibiting moderate superiority to H. At lesser degrees of toxicity, N₃ was superior to S(═O)Me and SO₂Me, which in turn were superior to the NHAc and phthalimido substitutions. The 5′ modifications findings with respect to the selectivity index paralleled those for the IC₅₀, with H, MeS and N₃ yielding higher SI than those for I, S(═O)Me, SO₂Me and those for NHAc and phthalimido.

2′- and 3′-Ribose Modification.

The possibility of improving cellular permeability or solubility was explored by comparison of the 2′- and 3′-OH group with 2′- and 3′-OAc substitution. The general trend is for the hydroxyl moiety to have lower IC₅₀'s than their acetyl counterparts, although there are examples of their equivalency. This finding can be understood if it is recalled that for the IAG hydrolases, the 5′- and 3′-hydroxyl groups contribute to catalysis and substrate binding, while the 2′-hydroxyl group contributes to catalysis only.⁵⁶ There are no substantive changes regarding the SI for OH versus OAc among the available comparators.

Modification of the C-6 Substituent for the Non-Cleavable 7-Deaza Guanosine Analogs.

The primary modification in this category is the replacement of the N-7 group with carbon, thereby rendering the nucleoside non-cleavable to the base via the purine nucleoside phosphorylase of the host. Only three modifications at the 6-position were examined, with oxygen being present for guanosine. The results show chlorine to be superior to sulfur or selenium. Of relevance, previous studies showed 6-thio-7-deaza-8-azapurine ribonucleoside to be less efficacious in vitro against T. brucei than 6-hydroxy-7-deza-8-azapurine ribonucleoside.³⁰ As for the IC₅₀, the SI for the C—I substituent as in ACT-91 is vastly superior to that for S or Se.

### CONCLUSIONS

These results provide proof of concept for the postulated selectivity of our nucleoside prodrugs in targeting protozoan pathogens while remaining relatively nontoxic to human hosts. To date, ACT-88 (81) has proven to be the most potent compound of those studied against P. falciparum (IC₅₀=110 nM, SI=1254), while ACT-91 (83) afforded the best results against T. brucei (IC₅₀=130 nM, SI=983) and L. donovani (IC₅₀=60 nM, SI=2717). ACT-51 (22) was the most effective agent against T. cruzi (IC₅₀=2.6 μM) and also showed strong activity against the other protozoa in our test panel. Unfortunately, it also proved relatively cytotoxic toward the L6 mammalian cell line (IC₅₀=584 nM). It is also noteworthy that the most active nucleosides shown in FIG. 2 compare very favourably with the drugs in current use listed as standards in the table for T. cruzi (benznidazole, IC₅₀=427 nM), L. donovani (miltefosine IC₅₀=122 nM) and P. falciparum (chloroquine IC₅₀=71 nM). On the basis of these results, compounds 81 and 83 appear to be the most promising leads for further screening in a suitable animal model. The N-nitrosoamine functionality of 81 raises concerns based on the mutagenicity associated with this general class, but further modification of the 6-position might provide new analogues with comparable antiprotozoan activity, but without the undesired effects. Finally, it is worth noting that this nucleoside-based approach has not been previously employed in any existing antiprotozoan drugs in clinical use. The novel mechanism suggests that the rapid development of resistance by the pathogens is less likely, especially since the drugs act on a fundamental genetic level that is essential for the propagation of protozoan pathogens.

### REFERENCES

1. World malaria report 2013. Geneva: World Health Organization, 2013
2. White, N. J. J. Clin. Invest. 2004, 113, 1084-1092
3. Ashley E. A.; Dhorda, M.; Fairhurst, R. M. et al. N. Eng. J. Med. 2014, 371, 411-423.
4. T. brucei http://www.ncbi.nlm.nih.gov/genome/?term=trypanosoma+brucei
5. Chagas, World Health Organization. Working to overcome the global impact of neglected tropical diseases. http://whqlibdoc.who.int/publications/2010/9789241564090_eng.pdf.
6. L. Donovani http://www.ncbi.nlm.nih.qov/Cqenome/?term=Leishmania+donovani
7. Marr, J. J.; Berens, R. L. J. Infect. Dis. 1977, 136, 724-732.
8. Sherman, I. W. Microbio. Rev. 1979, 43, 453-495.
9. Berens, R. I.; Krug, E. C.; Marr, J. J., In Biochemistry and Molecular Biology of Parasites, Marr, J. J.; Muller, M. eds. Academic Press, San Diego, Calif., 1995, pp. 323-336.
10. Parkin, D. W. J. Biol Chem. 1996, 271, 21713-21719.
11. Versees, W.; Decanniere, K.; Pellé, R.; Depoorter, J.; Brosens, E.; Parkin, D. W.; Steyaert J. J. Mol. Biol. 2001, 307, 1363-1379.
12. Berg, M.; Kohl, L.; Van der Veken, P.; Joossens, J; Al-Salabi, M. I.; Castagna, V.; Giannese, F.; Cos, P.; Versées, W.; Steyaert, J.; Grellier, P.; Haemers, A.; Degano, M.; Maes, L.; de Koning, H. P.; Augustyns, K. Antimicrob. Agents Chemother. 2010, 54, 1900-1908.
13. Savarese, T. M.; Kouni, M. H. Mol. and Biochem. Parasitology 2014, 194, 44-47.
14. El Kouni, M. H. Pharmacol. Therapeut. 2003, 99, 283-309.
15. Dadonna, P. E.; Wiesmann, W. P.; Milhouse, J. W.; Chern J. W.; Townsend, L. B.; Hershfield, M. S.; Webster, H. K. J. Biol. Chem. 1986, 261, 11667-11673.
16. Shi, W.; Ting, L.-M.; Kicska, G. A.; Lewandowicz, A.; Tyler, P. C.; Evans, G. B.; Furneaux, R. H.; Kim, K.; Almo, S. C.; Schramm, V. L. J. Biol, Chem. 2004, 279, 18103-18106.
17. Zimmerman, T. P.; Gersten, N. B.; Ross, A. F.; Meich, R. P. Can. J. Biochem. 1971, 49, 1050-1054.
18. Maynes, J. T.; Yam, W.; Jenuth, J. P. Gang, Y. R.; Litster, S. A.; Phipps, B. M.; Snyder, F. F. Biochem. J. 1999, 344, 585-592.
19. Ting, L. M.; Shi, W.; Lewandowicz, A.; Singh, V.; Mwakingwe, A.; Birck, M. R.; Ringia, E. A.; Bench, G.; Madrid, D. C.; Tyler, P. C.; Evans, G. B.; Furneaux, R. H.; Schramm, V. L.; Kim, K. J. Biol. Chem. 2005, 280, 9547-9554.
20. Huang, P.; Plunkett, W. Biochem. Pharmacol. 1987, 36, 2945-2950.
21. Silamkoti, A. V.; Allan, P. W.; Hassan, A. E. A.; Fowler, A. T.; Sorscher, E. J.; Parker, W. B.; Secrist, J. A. Nucleosides, Nucleotides and Nucleic Acids 2005, 24, 881-885.
22. Agarwal, R. P.; Sagar, S. M.; Parks, R. E. Biochem. Pharmacol. 1975, 24, 693-701.
23. Senft, A. W.; Crabtree, G. W.; Agarwal, K. C.; Scholar, E. M.; Agarwal, R. P.; Parks, R. E., Jr. Biochem. Pharmacol. 1973, 22, 449-458.
24. Miller, W. H.; Miller, R. L. Mol. Biochem. Parasitol. 1991, 48, 39-46.
25. Nelson, D. J.; LaFon, S. W.; Tuttle, J. V.; Miller, W. H.; Miller, R. L.; Krenitsky, T. A.; Elion, G. B.; Berens, R. L.; Marr, J. J. J. Biol. Chem. 1979 254, 11544-11549.
26. Mao, C; Cook, W. J.; Zhou, M.; Koszalka, G. W.; Krenitsky, T. A.; Ealick, S. E. Structure 1997, 5, 1373-1383.
27. Koellner, G; Bzowska, A; Wielgus-Kutrowska, B.; Lui6, M.; Steiner, T.; Saenger, W.; Stepiński, J. J. Mol. Biol. 2002, 315, 351-371.
28. LaFon, S. W.; Nelson, D. J.; Berens, R. L.; Marr, J. J. J. Biol. Chem. 1985, 260, 9660-9665.
29. Carson, D. A.; Chang, K. P. Biochem. Biophys. Res. Commun. 1981, 100, 1377-1383.
30. Fish, W. R.; Marr, J. J.; Berens, R. L.; Looker, D. L.; Nelson, D. J.; LaFon, S. W.; Balber, A. E. Antimicrob. Agents and Chemother. 1985, 27, 33-36.
31. Berens, R. L.; Marr, J. J. Biochem. Pharmacol. 1986, 35, 4191-4197.
32. Fei, X.; Wang, J-Q.; Miller, K. D.; Sledge, G. W.; Hutchins, G. D.; Zheng, Q-H. Nuclear Med. Bio. 2004, 31, 1033-1041.
34. Snyder, F.; Back, T. G. PCT Int. WO 2006/081665.
35. (a) A variation of the Vorbrüggen method was employed: Vorbrüggen, H.; Krolikiewicz, K.; Bennua, B. Chem. Ber. 1981, 114, 1234-1255. (b) For a survey of coupling methods, including the use of trimethylsilyl triflate under various conditions, for the synthesis of nucleosides, see: Vorbrüggen, H.; Ruh-Pohlenz, C. Handbook of Nucleoside Synthesis, J. Wiley and Sons, New York, 2001.
36. Barrett, A. G. M.; Lebold, S. A. J. Org. Chem. 1990, 55, 3853-3857.
37. (a) Ishikura, Y.; Kanazawa, T.; Sato, T. Bull. Chem. Soc. Jpn. 1962, 35, 731-735. (b) Townsend, L. B., Drach, J. C. U.S. Patent 1999/5874413.
38. Montgomery, J. A.; Shortnacy, A. T.; Thomas, H. J. J. Med. Chem. 1974, 17, 1197-1207.
39. Savarese, T. M.; Cannistra, A. J.; Parks, R. E., Jr.; Secrist, J. A., III; Shortnacy, A. T.; Montgomery, J. A. Biochem. Pharmacol. 1987, 36, 1881-1893.
40. Gillard, J. W.; Israel, M. Tetrahedron Lett. 1981, 22, 513-516.
41. Smellie, I. A.; Bhakta, S.; Sim, E.; Fairbanks, A. J. Org. Biomol. Chem. 2007, 5, 2257-2266.
42. Kuhn, R.; Werner, J.; Dietmann, K. U.S. Patent 1969/3475408.
43. van Tilburg, E. W.; von Frijtag Drabbe KOnzel, J.; de Groote, M.; Ijzerman, A. P. J. Med. Chem. 2002, 45, 420-429.
44. (a) Temple, C. Jr.; Kussner, C. L.; Montgomery, J. A. J. Org. Chem. 1966, 31, 2210-2215. (b) Czarnecki, J. J. Biochim. Biophys. Acta 1984, 800, 41-51. (c) Lioux, T.; Gosselin. G.; Mathé, C. Eur. J. Org. Chem. 2003, 3997-4002.
45. Compound 67 has been previously reported: Fishman, P.; Bar Yehuda, S.; Madi, L. PCT Int. Patent, WO 2005/111053.
46. The diazotization of 78 and 79 under various conditions produced mixtures of compounds that were difficult to separate. However, under the conditions indicated, only 80 and 81 could be isolated in pure form and modest yield in the 5′-deoxy- and 5′-methylthio series, respectively. It is likely that formation of the 2-fluoro-6-methylamino derivative 80 was accompanied by some of its corresponding N-nitroso derivative, while that of the N-nitroso compound 81 was accompanied by some of its 6-methylamino analogue, but these analogues could not be isolated or identified with certainty.
47. For previous preparations of 83 and 84, see: (a) Seela, F.; Soulimane, T.; Mersmann, K.; Jürgens, T. Helv. Chim. Acta 1990, 73, 1879-1887. (b) Ramasamy, K.; Imamura, N.; Robins, R. K., Revankar, G. R. J. Heterocyclic Chem. 1988, 25, 1893-1898.
48. Koszalka, G. W.; Krenitsky, T. A. In Purine and Pyrimidine Metabolism in Man, Nyhan, W. L.; Thompson, L. F.; Watts, R. W. E. (eds), Plenum Press, New York, N.Y., 1986, Part B, pp. 559-563.
49. Ghoda, L. Y.; Savarese, T. M.; Northup, C. H.; Parks, R. E., Jr.; Garofalo, J.; Katz, L.; Ellenbogen, B. B.; Bacchi, C. J. Mol. Biochem. Parasitol. 1988, 27, 109-118.
50. Savarese, T. M.; Harrington, S.; Nakamura, C.; Chen, Z. H.; Kumar, P.; Mikkilineni, A.; Abushanab, E.; Chu, S. H.; Parks, R. E., Jr. Biochem. Pharmacol. 1990, 40, 2465-2471.
51. Riscoe, M. K.; Ferro, A. J.; Fitchen, J. H. Parasitol. Today 1989, 5, 330-333.
52. Miller R. L.; Sabourin, C. L.; Krenitsky, T. A. Biochem. Pharmacol. 1987, 36, 553-559.
53. Shi, W.; Li, C. M.; Tyler, P. C. Biochem. 1999, 38, 9872-9880.
54. Marr, J. J.; Berens, R. L.; Cohn, N. K.; Nelson, D. J.; Klein, R. S. Antimicrob. Agents and Chemother. 1984, 25, 292-295.
55. Berman, J. D.; Hanson, W. L.; Lovelace, J. K.; Waits, V. B.; Jackson, J. E.; Chapman, W. L., Jr.; Klein, R. S. Antimicrob. Agents and Chemother. 1987, 31, 111-113.
56. Versées, W.; Decanniere, K.; Van Holsbeke, E.; Devroede, N.; Steyaert, J. J. Biol. Chem. 2002, 277, 15938-15946.
57. Baltz, T.; Baltz, D.; Giroud, C.; Crockett, J. EMBO J. 1985, 4, 1273-1277.
58. Desjardins, R. E.; Canfield, C. J.; Haynes, J. D.; Chulary, J. D. Antimicrob. Agents Chemother. 1979, 16, 710-718.
59. Vennerstrom, J. L.; Arbe-Barnes, S.; Brun, R.; Charman, S. A.; Chiu, F. C.; Chollet, J.; Dong, Y.; Dorn, A.; Hunziker, D.; Matile, H.; McIntosh, K.; Padmanilayam, M.; Santo Tomas, J.; Scheurer, C.; Scorneaux, B.; Tang, Y.; Urwyler, H.; Wittlin, S.; Charman, W. N. Nature 2004, 430, 900-904.
60. Werbovetz, K. A.; Sackett, D. L.; Delfin, D.; Bhattacharya, G.; Salem, M.; Obrzut, T.; Rattendi, D.; Bacchi, C. Mol. Pharmacol. 2003, 64, 1325-1333.
61. Werbovetz, K. A.; Brendle, J. J.; Sackett, D. L. Mol. Biochem. Parasitol. 1999, 98, 53-65.
62. O'Brien, J.; Wilson, I.; Orton, T.; Pognan, F. Eur. J. Biochem. 2000, 267, 5421-5426.
63. Srivastava, P. C.; Robins, R. K. J. Carbohydrates Nucleosides Nucleotides 1977, 4, 93-100.
64. Kissman, H. M.; Baker, B. R. J. Am. Chem. Soc. 1957, 79, 5534-5540.
65. Ginisty, M.; Gravier-Pelletier, C.; Le Merrer, Y. Tetrahedron Asymmetry 2006, 17, 142-150.
66. Although the present work was completed without incident, azides are well-known explosion hazards and should be handled with appropriate precautions.
67. Baker, B. R.; Kissman, H. M. U.S. Patent 1959/2875194.
68. Ohrui, H.; Misawa, T.; Meguro, H. J. Org. Chem. 1985, 50, 3007-3009.
69. Beigelman, L. N.; Mikhailov, S. N. Carbohydrate Res. 1990, 203, 324-329.
70. van Tilburg, E. W.; van der Klein, P. A. M.; von Frijtag Drabbe KOnzel, J.; de Groote, M.; Stannek, C.; Lorenzen, A.; Ijzerman, A. P. J. Med. Chem. 2001, 44, 2966-2975.
71. Bassi, L.; Gennari, F. Eur. Pat. Appl. (1993), EP 526866.
72. Milne, G. H.; Townsend, L. B. J. Heterocyclic Chem. 1971, 8, 379-382.
73. Zheng, Z.; Tran, H-A.; Manivannan, S.; Wen, X.; Kaiser, M.; Brun R.; Snyder F.; Back, T. G. (April 2016) Bioorganic & Medicinal Chemistry Letters 26:2861-2865.
74. Tran, H-A.; Zheng, Z.; Wen, X.; Manivannan, S.; Pastor, A.; Kaiser, M.; Brun R.; Snyder F.; Back, T. G. (February 2017) Bioorganic & Medicinal Chemistry 25:2091-2104.

We claim:

1. A compound of formula: 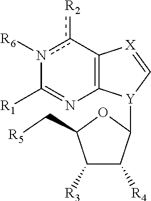
and salts thereof
wherein:
X and Y are independently selected from N, or CH;
R₁ is selected from the group consisting of a halogen, an amino group (—NH₂), an alkyl amino group (—N(R_{A})₂), an alkoxy group, a nitrosamino group (—N(R_{N})—NO), an imino group (—C(R_{C})=NR_{I}), an azide group, a cyano group, and a hydrazino group (—NH—N(R_{H})₂);
R₂ is selected from the group consisting of a halogen, an oxo (═O), a sulfhydryl (—SH), an amino group (—NH₂), an alkyl amino group (—N(R_{A})₂), an alkoxy group, a thioalkyl group, a nitrosamino group (—N(R_{N})—NO), an imino group (—C(R₁₀)=NR_{I}), an azide group, a cyano group, a hydrazino group (—N(R₁₀)—N(R_{H})₂), a hydroxyamino group (—N(R_{HA})OH), a ureido group (—N(R₁₀)—CO—N(R_{U})₂), an amido group (—N(R₁₀)—CO—R_{AD}), alkyl sulfinyl (—SO—R_{S}),
a 1-(1H)pyrrolyl group: 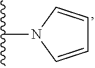 [SMILES: *n1cccc1]
a 1-(1H)-pyrazolyl group: 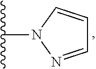 [SMILES: *n1cccn1]
and
a 1-(1H)-imidazolyl group; 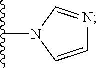
where R₆ is present only when R₂ is oxo;
each R₃ and R₄, independently, is a hydroxyl, or an acyl group (—COR_{AC});
R₅ is selected from the group consisting of hydrogen, hydroxyl, an alkyl, an alkoxy, an azido group, a sulfoxide group (—SO—R_{SO}), a sulfonyl group (—SO₂—R_{SO}), an amino group (—NH₂), an alkyl amino group (—N(R_{A})₂, a thioalkyl group (—SR_{T}), an amido group (—N(R₁₀)—CO—R_{AD}),
an N-phthalimido group: 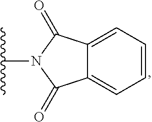 [SMILES: *N1C(=O)c2ccccc2C1=O]
and
a morpholino group: 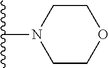
where
each R_{A}, R_{S} or R_{SO} is independently selected from an alkyl group having 1-6 carbon atoms, a cycloalkyl group having 3-6 carbon atoms and an alkyl having 1-3 carbon atoms;
each R₁₀, R_{C}, R_{H}, R_{I}, R_{HA}, R_{AD}, R_{AC}, R_{N}, R_{U}, and R_{T} is independently selected from hydrogen, an alkyl group having 1-6 carbon atoms, a cycloalkyl group having 3-6 carbon atoms and an alkyl having 1-3 carbon atoms; and
R₆ is selected from the group consisting of hydrogen and an alkyl group having 1-3 carbon atoms
or more specifically a methyl, ethyl, propyl or a cyclopropyl group or more specifically a methyl, ethyl, propyl or a cyclopropyl group

2. The compound of claim 1, wherein
(1) R₂ is oxo and R₆ is present or
(2) R₂ is a group other than oxo and R₆ is not present.

3. The compound of claim 1, wherein:
(1) Y is N;
(2) X is CH;
(3) each X and Y is CH; or
(4) each X and Y is N.

4. The compound of claim 1, wherein R₁ is F.

5. The compound of claim 1, wherein R₁ is F and each X and Y is N.

6. The compound of claim 1, wherein R₁ is F, each X and Y is N and each R₃ and R₄ is a hydroxyl group.

7. The compound of claim 1, wherein R₂ is selected from an alkyl amino group (—N(R_{A})₂), an azide group, a hydrazino group (—N(R₁₀)—N(R_{H})₂), a hydroxyamino group (—N(R_{HA})OH), a ureido group (—N(R₁₀)—CON(R_{U})₂), an amido group (—N(R₁₀)—CO—R_{AD}), an alkyl sulfinyl (—SO—R_{S}) group, a 1-(1H)pyrrolyl group, a 1-(1H)-pyrazolyl group, and a 1-(1H)-imidazolyl group.

8. The compound of claim 1, wherein R₅ is hydrogen or a thioalkyl group.

9. The compound of claim 1, wherein R₅ is a thioalkyl group.

10. The compound of claim 1, wherein R₅ is a thiomethyl group.

11. The compound of claim 1, wherein R₁ is an amino group.

12. The compound of claim 11, wherein:
(1) each X and Y is N;
(2) X is N and Y is CH;
(3) X is CH and Y is N;
(4) X is CH; Y is N; and each R₃ and R₄ is a hydroxyl group;
(5) X is N; Y is CH; and each R₃ and R₄ is a hydroxyl group;
(6) X is CH; Y is N; and each R₃, R₄, and R₅ is a hydroxyl group;
(7) X is N; Y is CH and each R₃, R₄, and R₅ is a hydroxyl group; or
(8) R₂ is a halogen or a sulfhydryl group.

13. A compound of the formula: 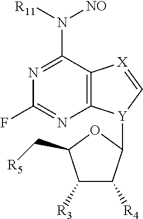
wherein:
X and Y are independently selected from N, or CH;
R₁₁ is an alkyl group having 1-3 carbon atoms;
each R₃ and R₄, independently, is a hydroxyl, or an acyl group (—COR_{AC});
R₅ is selected from the group consisting of hydrogen, hydroxyl, an alkyl, an alkoxy, an azido group, a sulfoxide group (—SO—R_{SO}), a sulfonyl group (—SO₂—R_{SO}), an amino group (—NH₂), an alkyl amino group (—N(R_{A})₂, a thioalkyl group (—SR_{T}), an amido group (—N(R₁₀)—CO—R_{AD})),
an N-phthalimido group: 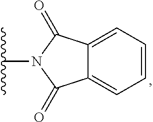 [SMILES: *N1C(=O)c2ccccc2C1=O]
and
a morpholino group: 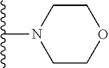
where
each R_{A}, R_{S} or R_{SO} is independently selected from an alkyl group having 1-6 carbon atoms, or a cycloalkyl group having 3-6 carbon atoms; and
each R_{AD}, R_{T}, or R_{AC} is independently selected from hydrogen, an alkyl group having 1-6 carbon atoms, or a cycloalkyl group having 3-6 carbon atoms.

14. The compound of claim 13, wherein:
(1) each X and Y is N;
(2) X is CH and Y is N;
(3) X is N and Y is CH;
(4) each R₃ and R₄ is a hydroxyl group;
(5) R₁₁ is a methyl group; or
(6) R₅ is a thioalkyl group.

15. The compound of claim 1, which is

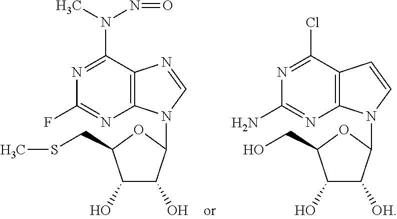 [SMILES: CSC[C@H]1OC(n2cnc3c(N(C)N=O)nc(F)nc32)[C@H](O)[C@@H]1O ; Nc1nc(Cl)c2ccn(C3O[C@H](CO)[C@@H](O)[C@H]3O)c2n1]

16. The compound of claim 1, wherein
(1) when R₂ is a monoalkyl amino group and R₁ is a halogen, then R₅ is a group other than alkoxy or thioalkyl;
(2) when R₁ is a halogen and R₅ is an alkoxy or thioalkyl group, then R₂ is a group other than a monoalkyl amino group: or
(3) when R₂ is a monoalkyl amino group and R₅ is an alkoxy or thioalkyl group, then R₁ is a group other than a halogen.

17. A pharmaceutical composition for the treatment of a bacterial or protozoan infection comprising as active ingredient a therapeutically effective amount of one or more compounds, salts, hydrates or solvates of claim 1 and a pharmaceutically acceptable carrier or diluent.

18. A method of treating a protozoan infection in a mammal in need thereof which comprises administering to said mammal a therapeutically effective amount of any one or more of the compounds, salts, hydrates or solvates of claim 1.

19. A method of inhibiting the growth of a protozoa in a mammal comprising administering to said mammal a growth-inhibiting effective amount of any one or more compounds, salts, hydrates or solvates of claim 1.
